(19) United States
(12) Patent Application Publication
(10) Pub. No.: US 2013/0261317 A1
(43) Pub. Date: Oct. 3, 2013

(54) METHODS FOR PREPARING SYNTHETIC BILE ACIDS AND COMPOSITIONS COMPRISING THE SAME

(71) Applicants: Robert M. Moriarty, Michiana Shores, IN (US); Photon Rao, Foster City, CA (US)

(72) Inventors: Robert M. Moriarty, Michiana Shores, IN (US); Photon Rao, Foster City, CA (US)

(73) Assignee: KYTHERA BIOPHARMACEUTICALS, INC., Calabasas, CA (US)

(21) Appl. No.: 13/876,069

(22) PCT Filed: Sep. 19, 2011
(86) PCT No.: PCT/US2011/052204

§ 371 Date: Jun. 18, 2013

Related U.S. Application Data
(60) Provisional application No. 61/386,944, filed on Sep. 27, 2010.
(60) Provisional application No. 61/527,034, filed on Aug. 24, 2011.

(51) Int. Cl. C07J 41/00; C07J 9/00; C07J 21/00

(52) U.S. Cl. CPC C07J 41/0061; C07J 21/006; C07J 9/005
USPC 549/334; 552/549; 552/551; 552/552; 552/550

(57) ABSTRACT

This invention relates generally to methods for preparing certain bile acids from non-mammalian sourced starting materials as well as to synthetic bile acids and compositions comprising such acids wherein the acids are characterized by a different C¹⁴ population than naturally occurring bile acids as well as being free from any mammalian pathogens. This invention is also directed to the synthesis of intermediates useful in the synthesis of such bile acids. Accordingly, the C ring of the steroidal scaffold is oxidized to provide a synthetic route and intermediates to DCA. This invention also provides synthetic methods for preparing deoxycholic acid or a salt thereof starting from aromatic steroids such as estrogen, equilenin, and derivatives thereof. This invention is also directed to intermediates such as 12-oxo or delta-9,11-ene steroids as well as novel processes for their preparation. In preferred embodiments, bile acids are provided herein which have substituents on the B-ring and/or D-ring side chain and optionally on the hydroxy group of the A-ring.

### FIELD OF THE INVENTION

This invention relates generally to methods for preparing certain bile acids from non-mammalian sourced starting materials as well as to synthetic bile acids and compositions comprising such acids. In some cases, the acids are characterized by a different C¹⁴ population than naturally occurring bile acids. Importantly, the bile acids of the present invention are not isolated from mammals and microbial organisms naturally producing these acids and thus are free of any toxins and contaminants associated with such organisms. This invention is also directed to novel intermediates of bile acids and methods of making them. Accordingly, the C ring of a steroidal scaffold, preferably that of an aromatic or an A,B-trans steroid, is oxidized to provide synthetic routes and intermediates to bile acids. Thus, e.g., this invention provides synthetic methods for preparing a bile acid or a salt thereof starting from aromatic steroids such as estrogen, equilenin, equilin and derivatives thereof. This invention is also directed to intermediates such as 12-oxo or delta-9,11-ene steroids as well as novel processes for their preparation. In preferred embodiments, bile acids are provided herein which have substituents on the B-ring and/or D-ring side chain and optionally on the hydroxy group of the A-ring.

### BACKGROUND OF INVENTION

Bile acids are important biological molecules. They act as emulsifying agents for dietary fats by forming mixed micelles. Bile acids solubilize lipids such as vitamin D and vitamin E.

The chemical structures of certain bile acids and conjugates thereof, and the biosynthetic pathway for various bile acids in mammals are provided below in Schemes 1 and 2.

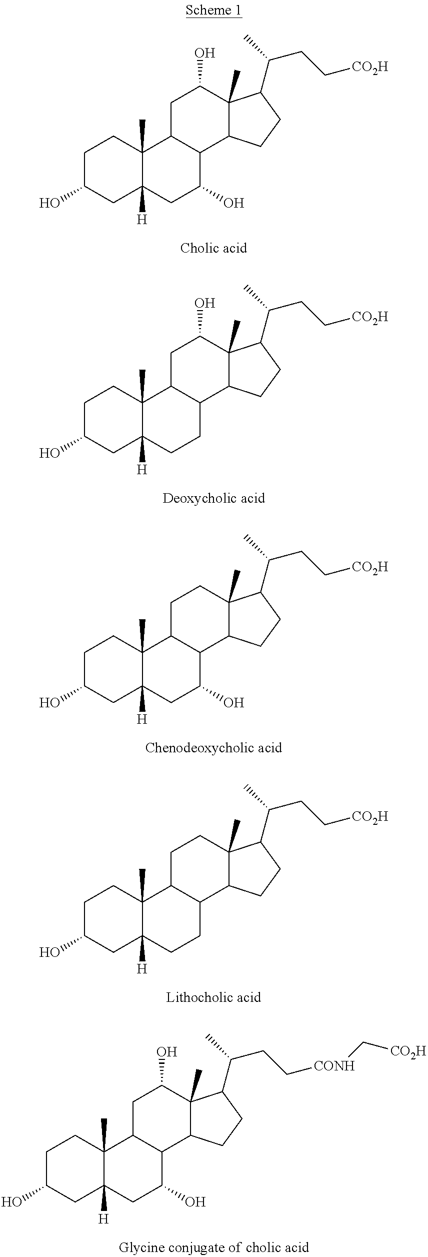

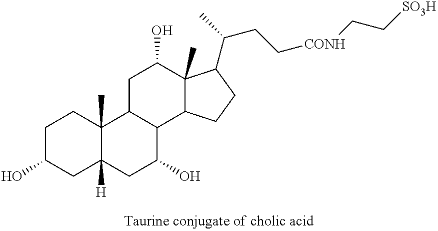

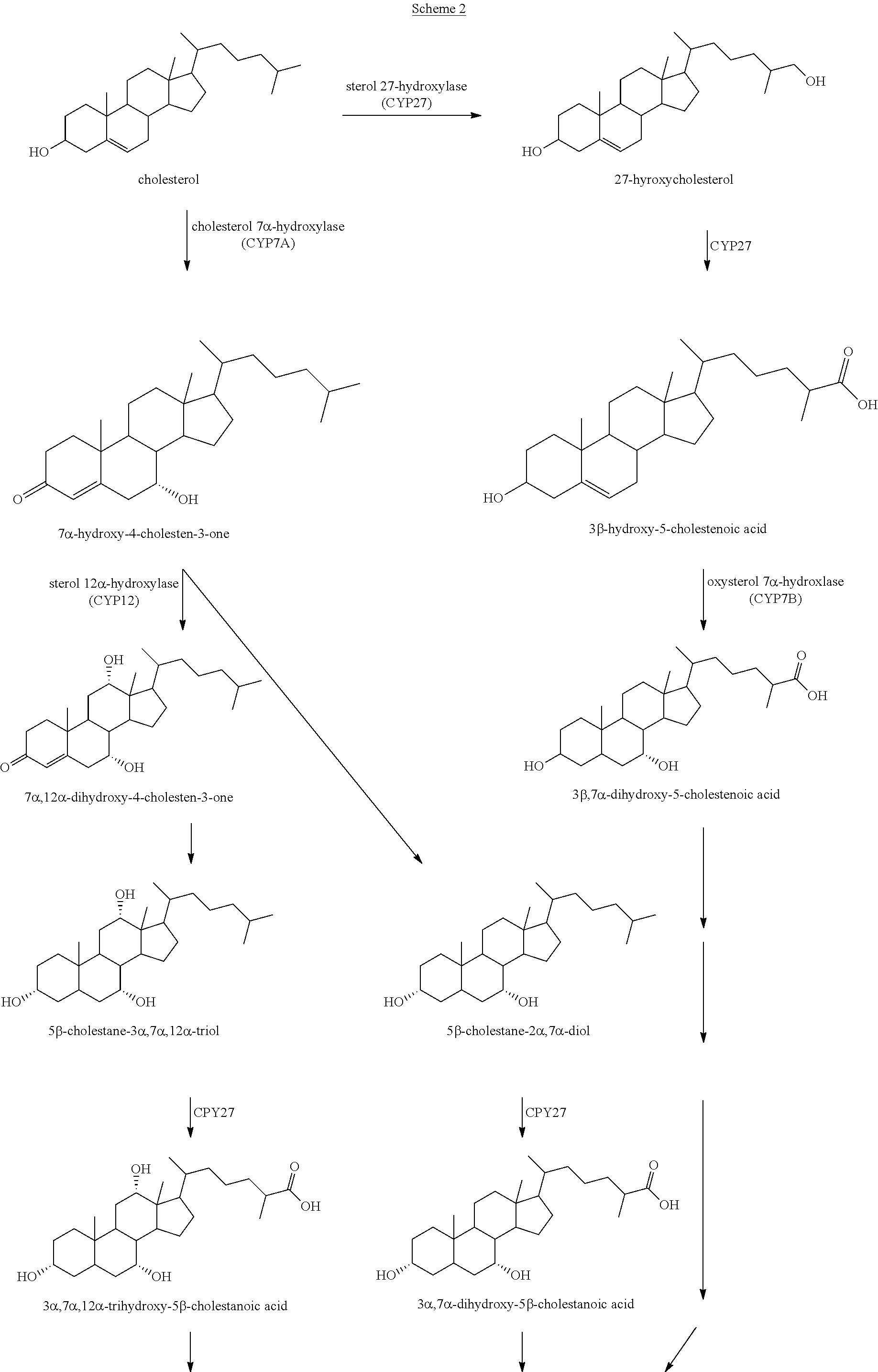

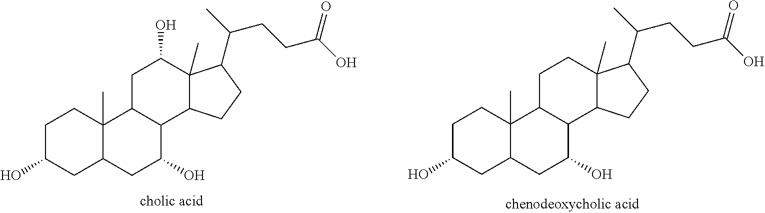 [SMILES: CC(CCC(=O)O)C1CCC2C3C(C[C@H](O)C12C)C1(C)CC[C@@H](O)CC1C[C@H]3O ; CC(CCC(=O)O)C1CCC2C3C(CCC12C)C1(C)CC[C@@H](O)CC1C[C@H]3O]

Bile acids have received attention for various therapeutic uses. They act as transport systems for drugs targeted for the liver. They also improve intestinal absorption of peptide based drugs. Bile acid derivatives exhibit antiviral and antifungal activity and are also used as drug carriers to allow poorly bioabsorbed drugs to pass through the intestinal walls. See, for example, Cundy, et al., U.S. Pat. No. 6,900,192 and Cundy, et al., U.S. Pat. No. 6,992,076, both of which are incorporated herein by reference in their entirety.

Recently published literature reports that deoxycholic acid has fat removing properties when injected into fatty deposits in vivo. See, WO 2005/117900 and WO 2005/112942, as well as U.S. 2005/0261258; U.S. 2005/0267080; U.S. 2006/127468; and U.S. 2006/0154906, all incorporated herein by reference in their entirety including figures. While pharmaceutical grade bile acid preparations are commercially available at relatively low cost, this low cost is due to the fact that the bile acids are obtained from animal carcasses, particularly large animals such as cows and sheep.

Notwithstanding such common availability, many countries prefer to use synthetically derived products rather than animal derived products and require that if a synthetic product is available, it must be used in place of the animal derived product. Accordingly, processes and intermediates for the preparation of synthetic bile acids are desired. This invention addresses this issue by providing synthetically prepared bile acids. The disclosed bile acid compositions can be used in adipolytic therapy and will serve to further advance research and developmental efforts in the area of localized fat removal.

There is a need to develop synthetic routes to bile acids to provide bile acids that are free of mammalian or microbial pathogens as well as free of any compounds related to the biosynthesis of bile acid, specifically, deoxycholic acid, cholic acid, chenodeoxycholic acid and lithocholic acid that is free of intermediates or other bile acids formed upstream of their respective productions, as described in Scheme 2. In this regard, GB Patent No. 2452358 provides one synthetic route for the synthesis of deoxycholic acid and salts thereof.

### SUMMARY OF THE INVENTION

This invention is directed to bile acids or salts thereof prepared by synthetic methods not employing mammalian sourced starting materials. This invention is also directed to methods for preparing synthetic bile acids or salts thereof as well as compositions comprising such acids or salts. Importantly, since the bile acids of this invention are not isolated from mammalian sources, they are thus free of any toxins and contaminants associated with such mammals.

Also provided herein are synthetic methods for making deoxycholic acid (DCA), cholic acid (CA), and other bile acids, and salts of each thereof. Also provided herein are compounds that are intermediates useful in these synthetic methods.

In one aspect, the synthetic methods comprise employing an aromatic steroid as a starting material or as an intermediate in at least one synthetic step. In one embodiment, the aromatic steroid thus employed is of formula:

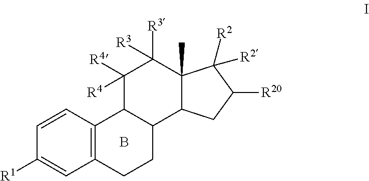

wherein ring B is of formula:

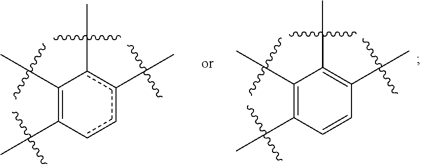

wherein [symbol] is either a single or a double bond provided that no two adjacent bonds can both be a double bond (i.e., the two adjacent bonds can not form an allenic double bond);

R¹ is OH, —OR¹¹, or —OCOR¹²;

R¹¹ is substituted or unsubstituted alkyl, alkenyl, or alkynyl;

R¹² is H, substituted or unsubstituted alkyl, alkenyl, alkynyl, or aryl;

R² and R^{2′} independently are H, substituted or unsubstituted alkyl, alkenyl, or alkynyl, or are —COR²², —OR²², —OCOR²²; or R² and R^{2′} together with the carbon atom they are bonded to form a cyclic ketal, or CR²R^{2′} is oxo, C═CR²³R²⁴, or is a complexed or uncomplexed ligand, which is at least bidentate and chelates via at least two heteroatoms selected from nitrogen, oxygen, sulfur, or phosphorous;

R²⁰ is H or R²⁰ and R² together with the carbon atom they are bonded to form an epoxide or a double bond;

R²² is H or substituted or unsubstituted alkyl, alkenyl, alkynyl, or aryl;

R²³ and R²⁴ are independently H or substituted or unsubstituted alkyl;

R³ and R^{3′} independently are H, OH, substituted or unsubstituted alkyl, alkenyl, or alkynyl, or are —OR³¹, —OCOR³¹; or R³ and R^{3′} together with the carbon atom they are bonded to form a cyclic ketal, or CR³R^{3′} is oxo;

R³¹ is substituted or unsubstituted alkyl; and

R⁴ and R^{4′} independently are H or OH, or CR⁴R^{4′} is oxo.

In one embodiment, the aromatic steroid thus employed is of formula:

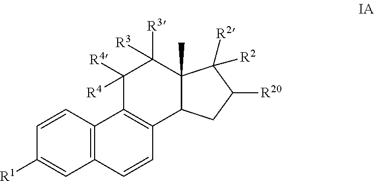

wherein R¹, R², R^{2′}, R³, R^{3′}, R⁴, R^{4′} and R²⁰ are defined as above.

In another embodiment, the aromatic steroid thus employed is of formula:

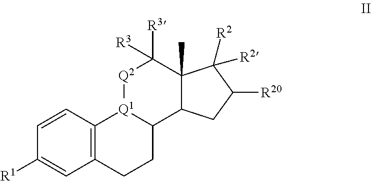

wherein Q¹-Q² is C═CH or CH—CH₂, and R¹, R², R^{2′}, R³, R^{3′}, and R²⁰ are defined as above.

In one embodiment, the method comprises contacting under reducing conditions the aromatic steroid of formula I or II to reduce one or both of the aromatic rings of the aromatic steroids. In one embodiment, the reducing is performed under Birch reduction conditions. Under the Birch reduction conditions as useful in this invention, the compound of formula I or II is contacted with at least 4 equivalents of an alkali metal in liquid ammonia and at least 4 equivalents of an alcohol, optionally in a solvent. Suitable alkali metals include lithium and sodium. Suitable alcohols include ethanol and tertiary butyl alcohol. Suitable optional solvents include inert solvents such as diethyl ether. The contacting is carried out for a period of time to yield a substantial amount of the product. In another embodiment, the product thus obtained is of formula:

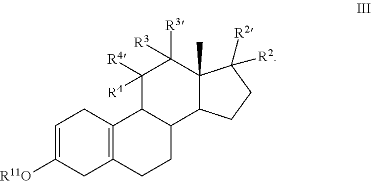

In another embodiment, the method comprises contacting a compound of formula III with a carbene of formula CX₂ or a precursor thereof, wherein each X is independently halo or hydrogen, under carbene forming conditions to provide the compound of formula:

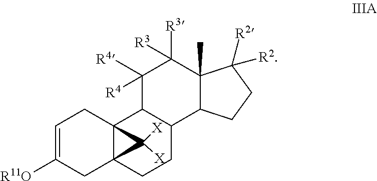

Preferred carbene forming conditions useful in this invention include, without limitation, reacting a haloform with a strong base, such as tertiary butoxide, and Simmons Smith reaction conditions (employing diiodomethane and zinc copper couple). Suitable carbine precursors include haloforms, diidodomethane, and the like. At least 1 equivalent, preferably, at least 3-4 equivalent of the haloform is employed. A preferred haloform is bromoform. Suitable inert solvents for performing the dihalocarbene insertion include, diethyl ether, pentane, and the like. The reaction is carried out at −30° C. to 10° C., for a period of time to yield a substantial amount of the product. This reaction can also provide the bis carbene adduct, which can be converted according to the methods described here to 2-substituted, such as 2 methyl bile acid derivatives.

In another embodiment, the method comprises contacting the Birch reduction product, III, under ketalization conditions to provide a compound of formula IIIE:

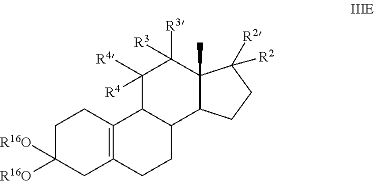

wherein R¹⁶ is substituted or unsubstituted alkyl, alkenyl, or alkynyl, or two R¹⁶ groups together with the oxygen atoms they are attached to form a cyclic ketal. Preferred ketalization conditions useful in this invention include, without limitation, refluxing an alcohol or a diol, in the presence of an acid, and may include water removal, such as by distillation. Suitable alcohols include methanol, ethanol, and the like. Suitable diols include ethylene glycol, propylene glycol, and the like. Suitable acids include, para toluenesulfonic acid, HCl gas, and the like. Inert solvents such as anhydrous diethyl ether and such other anhydrous solvents may be used as cosolvents. At least 2 equivalent of the alcohol, or at least 1 equivalent of the diol is used; preferably the alcohol or the diol is used in excess. Molecular sieves are also useful to remove water in this step. The contacting is performed for a period of time to yield a substantial amount of the product. Preferably, R¹⁶ is unsubstituted alkyl, or two R¹⁶ groups together with the oxygen atoms they are attached to form a 5 or 6 membered cyclic ketal.

In another embodiment, the method comprises contacting a compound of formula IIIE with a carbene of formula CX₂ or a precursor thereof, wherein each X is independently halo or hydrogen, under carbene forming condition, such as those described above, to provide the compound of formula:

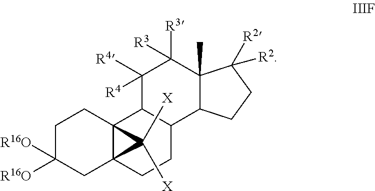

In another embodiment, the method optionally comprises reducing the compound of formula IIIA to provide the compound of formula:

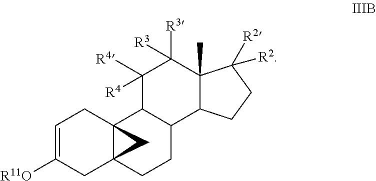

In another embodiment, the method optionally comprises contacting the compound of formula IIIF under reducing conditions, to provide the compound of formula:

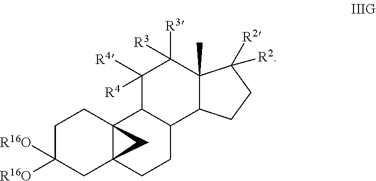

The reducing steps are necessary if one of the X groups is a halo group. This reduction can be performed, preferably under Birch reduction conditions as described. Catalytic hydrogenation may also be employed using supported (on carbon, alumina, and the like) palladium, platinum, rhodium, or such other metals, or their oxides and hydroxides as a hydrogenation catalyst.

In another embodiment, the method comprises contacting the compound of formula IIIB or IIIG, or the compound of formula IIIA wherein X is H, with an acid to provide the compound of formula:

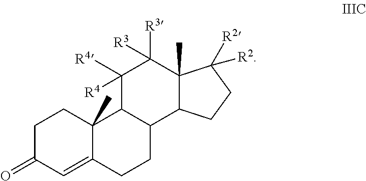

At least 1 equivalent of the acid is employed. Suitable acids include anhydrous HCl and the like. The contacting is carried out in an inert solvent, including without limitation chloroform. The contacting is carried out at a temperature of 5° C.-45° C., for a period of time to provide a substantial amount of the product.

In another embodiment, R^{2′} is H and R² is hydroxy, substituted or unsubstituted alkyl, alkenyl, or alkynyl, or is —OR²², —COR²², or —OCOR²²; or R² and R^{2′} together with the carbon atom they are bonded to form a cyclic ketal, or CR²R^{2′} is oxo or C═CR²³R²⁴. In another embodiment, R²² is alkyl. In another embodiment, R²² is a hydroxy substituted alkyl. In another embodiment, R²² is methyl. In another embodiment, R²² is —CH(OH)CH₃. In another embodiment, R^{3′} is H and R³ is hydroxy, —OR³¹, or —OCOR³¹; or R³ and R^{3′} together with the carbon atom they are bonded to form a cyclic ketal, or CR³R^{3′} is oxo. In another embodiment, R^{3′} is H, and R³ is an alpha or beta hydroxy, OR³¹, or is —OCOR³¹. In another embodiment, R³¹ is methyl, ethyl, allyl, benzyl, or the like. In another embodiment, R⁴ and R^{4′} are H.

Methods of converting a compound of formula IIIC to a compound of formula:

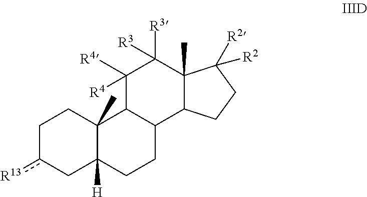

wherein [symbol] is either a single or a double bond; R¹³ is R¹ or O, provided that when R¹³ is bonded to the 3-position by a double bond, then R¹³ is O; and R², R^{2′}, R³, R^{3′}, R⁴, and R^{4′} are defined as is in any aspect and embodiment herein, are well known to the skilled artisan. See, e.g., U.S. patent application publication no. 2010/0160276, which is incorporated herein by reference.

In one embodiment, the method comprises at least one step wherein a steroid is hydroxylated at the 12 position comprising contacting a steroid of formula I or IV

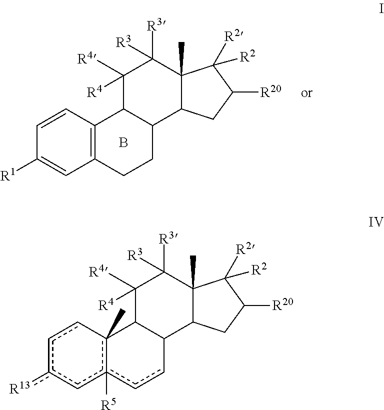

wherein [symbol] is either a single or a double bond provided that no two adjacent bonds can both be a double bond (i.e., the two adjacent bonds can not form an alleneic double bond);

R¹³ is R¹ or O, provided that, when R¹³ is bonded to the steroid scaffold with a double bond, then R¹³ is O;

R¹ is defined as in any embodiment herein, and preferably is —OR¹¹ or —OCOR¹²;

CR²R^{2′} is of formula:

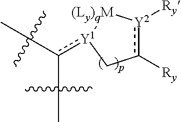

R²⁰ is H;

p is 0, 1, 2, or 3;

q is 0, 1, 2, 3, 4, or 5;

Y¹ and Y² independently are nitrogen, oxygen, sulfur, or phosphorous;

R_{y} and R_{y}′ independently are H, or substituted or unsubstituted alkyl, alkenyl, alkynyl, aryl, heteroaryl, heterocycle, or cycloalkyl; or R_{y} and R_{y}′ together with the carbon and heteroatom they are bonded to form a substituted or unsubstituted heterocycle or heteroaryl;

M is a metal selected from copper, manganese, iron, chromium, cobalt, and the like containing +1 to +6 charge; and

L^{y} is an anion having a charge of −1 to −6 and/or is a neutral ligand;

R³, R^{3′}, R⁴, and R^{4′} are H; and

R⁵ is absent or is alpha H, beta H, or is a mixture of alpha and beta H, provided that when the 5-position of the steroid is an SP² carbon, then R⁵ is absent, and when the 10-position of the steroid is an SP² carbon, then the 19-angular methyl is absent; with oxygen or another similar oxidizing agent, to provide a compound of formula I or IV wherein R³ is hydroxy and the other substituents are as defined for the starting material I or IV.

In one embodiment, the moiety:

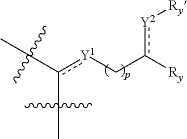

is of formula:

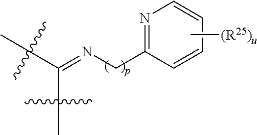

wherein p is 1 or 2 and each R²⁵ independently is H or substituted or unsubstituted alkyl or aryl.
In another embodiment, M is copper. In another embodiment, the copper has a charge of +1 to +3. In another embodiment, the oxidizing agent is oxygen. In another embodiment, L^{y} is triflate. A preferred method of hydroxylating the 12-position is described in the Examples section below.

In another embodiment, provided is a method comprising contacting a compound of formula I or IV:

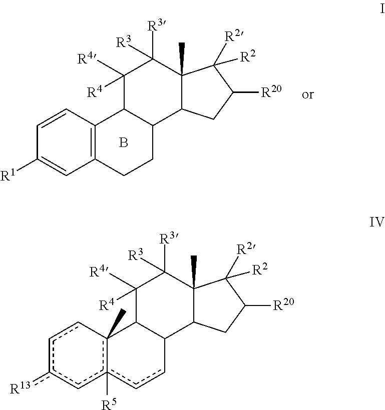

wherein CR²R^{2′} is oxo or R² and R^{2′} are together a cyclic ketal, R²⁰ is H; R^{3′} is H, R³ is hydroxy, and R⁴ and R^{4′} are H; with an oxidizing agent under an oxidizing condition to provide a compound of formula I or IV, wherein CR³R^{3′} is oxo. A variety of oxidizing agents and oxidizing conditions well known to the skilled artisan is useful to perform this oxidation. Within these embodiments, R¹ and R⁵ are defined as in any aspect or embodiment herein, and preferably, R¹ is —OR¹¹ or OCOR¹². In another embodiment, provided is a method comprising contacting the compound of formula I or IV, wherein CR³R^{3′} is oxo, with a alcohol or a diol under ketalization conditions to provide a compound of formula I or IV, wherein R³ and R^{3′} are —OR³¹ or R³ and R^{3′} together with the carbon atom they are bonded to form a ketal.
In one embodiment, the compound of formula IV:

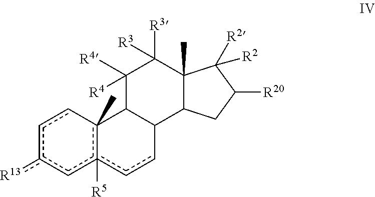

is a compound of formula:

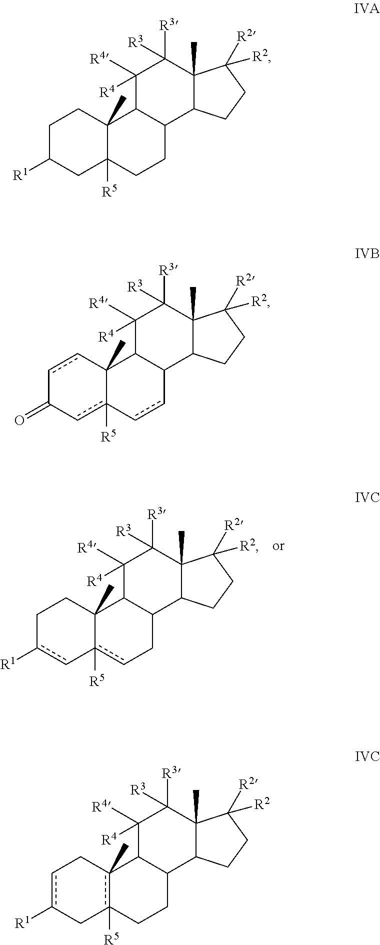

wherein R¹, R², R^{2′}, R³, R^{3′}, R⁴, R^{4′}, and R⁵ are defined as in any aspect or embodiment herein. In another embodiment, the compound of formula IV is a compound of formula:

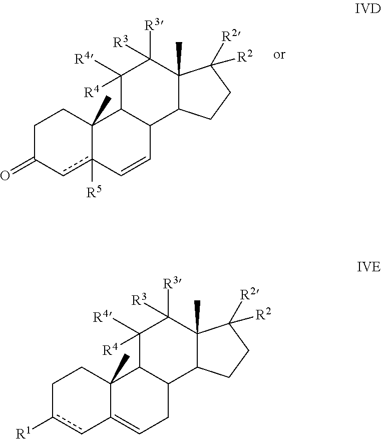

wherein R¹, R², R^{2′}, R³, R^{3′}, R⁴, R^{4′}, and R⁵ are defined as in any aspect or embodiment herein.

In another embodiment, provided is a method comprising contacting the compound of formula:

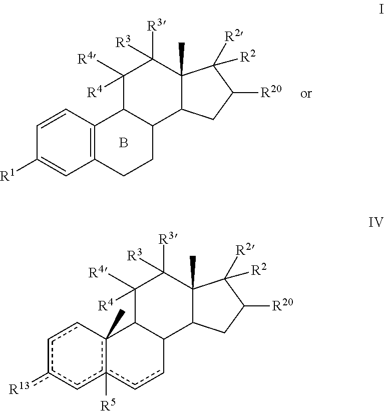

wherein CR³R^{3′} is oxo, and wherein R¹, R², R^{2′}, R⁴, R^{4′}, R⁵, R¹³, and R²⁰ are defined as in any aspect or embodiment herein with a reducing agent under reducing conditions to provide a compound of formula I or IV, wherein R^{3′} is H and R³ is alpha hydroxy. A variety of reducing agents and reducing conditions well known to the skilled artisan, and provided herein, are useful to perform this reduction. Preferably, the reducing is performed employing LiAlH(O^{t}Bu)₃.

In another embodiment, the method comprises reacting the compound of formula I or IV wherein R^{3′} is H and R³ is alpha hydroxy with a protecting group, to protect the R³ hydroxy group. In another embodiment, the protected compound is a compound of formula I or IV wherein R^{3′} is H, R³ is alpha —OR³¹.

A method for preparing alkyl ethers from 3, 11, 12, or 17 hydroxy group of the compounds utilized herein employs alkyl or substituted alkyl trichloroacetamidates and an acid. Such alkyl or substituted alkyl trichloroacetamidates are commercially available and are easily prepared from trichloroacetonitrile and the corresponding alkoxide. Commercially available acetamidates include, without limitation, methyl, allyl, benzyl, and 4-methyoxybenzyl trichloroacetamidate.

In another embodiment, the method comprises at least one step comprising contacting a steroid of formula:

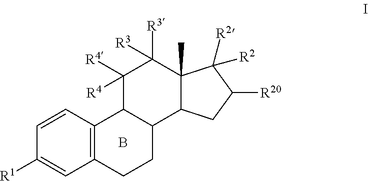

wherein ring B is:

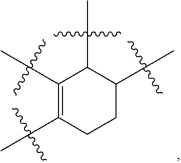 [SMILES: *C1=C(*)C(*)C(*)CC1]

with an oxidizing agent, or in other words, hydroxylating a steroid of formula

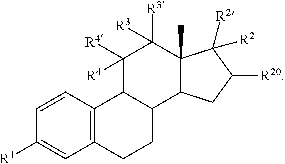

within this embodiment, R¹ is defined as in any aspect or embodiment herein; R² and R^{2′} independently are H, hydroxy, substituted or unsubstituted alkyl, alkenyl, alkynyl, or aryl, or are —OR²², —COR²², —OCOR²²; or R² and R^{2′} together with the carbon atom they are bonded to form a ketal, or CR²R^{2′} is oxo or C═CR²³R²⁴; R²², R²³, and R²⁴ are defined as in any aspect or embodiment herein; R²⁰ is H or R²⁰ and R² together with the carbon atom they are bonded to form an epoxide; and R³, R^{3′}, R⁴, and R^{4′} are H;
to provide a compound of formula:

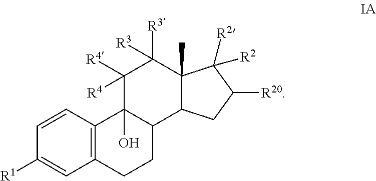

In another embodiment, the method comprises hydroxylating a steroid of formula:

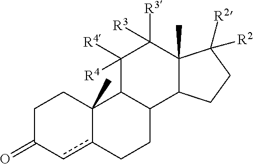

wherein R², R^{2′}, R³, R^{3′}, R⁴, and R^{4′} are defined as in any aspect or embodiment herein, under microbial oxidation conditions to provide a compound of formula:

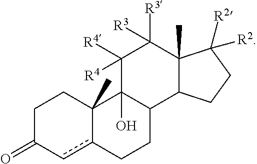

Enzymes suitable for carrying out such transformations include, without limitation, 3-ketosteroid 9α-hydroxylase A and B, as found, for example, and without limitation, in Rhodococcus species. In another embodiment, the microorganism employed is Nocardia canicruria ATCC 31548. Microorganism of the genus Mycobacterium, such as the Mycobacterium species NRRL-B-3805 is also useful for such 9-hydroxylation. Preferably, CR²R^{2′} is oxo. More preferably, CR²R^{2′} is oxo and R³, R^{3′}, R⁴, and R^{4′} are H. Similarly, steroid derivates, for example, and without limitation those having a one or more of a 3-oxo, a 16-oxo, and a 17-, are also hydroxylated at the 11, and 12 positions of the steroid scaffold following microbial oxidation, employing, for example, Rhizopus arrhizus or Rhizopus nigricans. When available for oxidation, the 3-position of the steroid can also be microbially oxidized. See also, Jones, Pure Appl. Chem., 1973, 29-52. Such hydroxylated steroids are elaborated, according to the methods disclosed herein, to bile acid derivatives.

In one embodiment, R² is H, and R^{2′} is hydroxy, substituted or unsubstituted alkyl, or alkoxy, or is —COR²², —OCOR²⁵; or R² and R^{2′} together with the carbon atom they are bonded to form a ketal, or CR²R^{2′} is oxo. In another embodiment, R² is H, and R^{2′} is COR²². In another embodiment, R²² is alkyl. In another embodiment, R²² is methyl. Preferably, the oxidizing agent is persulfate or dioxirane. The 9-hydroxylation is performed by contacting at least 1 equivalent of the oxidizing agent in an inert solvent at a temperature of −10° C. to 10° C. for a period of time to provide a substantial amount of the 9-hydroxylated steroid. Suitable solvents include, without limitation dichloromethane and the like.

In another embodiment, the method comprises, subjecting the compound of formula:

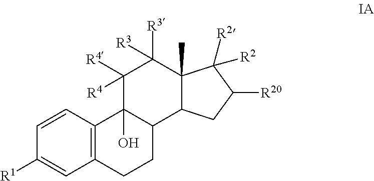

to dehydration conditions to provide the compound of formula:

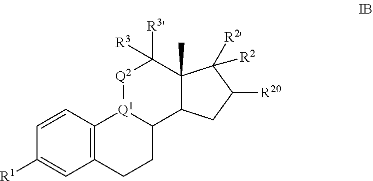

wherein Q¹-Q² is C═CH. Reagents and dehydrating conditions for performing this reaction are well known to the skilled artisan.

In another embodiment, provided is a method comprising contacting a compound of formula IB with an oxidizing agent under to provide a compound of formula IB, wherein CR³R^{3′} is oxo, or R³ is H and R^{3′} is hydroxy or is —OOR³² and R³² is H or alkyl. In one embodiment, R³² is H or tertiary butyl. Preferably, the oxidizing agent is a copper or a chromium oxidizing agent. More preferably, the oxidizing agent is an alkyl hydroperoxide such as tertiary butyl hydroperoxide, and a hypohalite or a copper or a chromium oxidizing agent. The reaction is carried out in an inert solvent, including without limitation ethyl acetate, for a period of time to provide a substantial amount of the product. The reaction is carried out at −10° C.-15° C.

In another embodiment, provided is a method comprising contacting the compound of formula IB, wherein CR³R^{3′} is oxo, with a reducing agent under reducing conditions, to provide a compound of formula IB wherein Q¹-Q² is CH—CH₂ and/or a compound wherein Q¹-Q² is CH—CH₂, R^{3′} is H, and R³ is alpha hydroxy. The reducing agent is preferably hydrogen, and contacting is performed in the presence of a hydrogenation catalyst and an inert solvent. At least 1 equivalent of hydrogen is employed. Suitable solvents include, ethanol, methanol, ethyl acetate, diethyl ether, and the like. The reaction is carried out at 40° C.-60° C. for a period of time to provide a substantial amount of the product.

In another embodiment, the method comprises contacting the compound of formula IB, wherein R¹ is defined as in any aspect or embodiment herein, Q¹-Q² is CH—CH₂ or C═CH with a reducing agent under reducing conditions, preferably under Birch reduction conditions, to provide a dearomatized compound of formula:

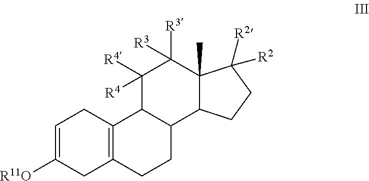

wherein R¹ is —OR¹¹ or —OCOR¹² wherein R¹¹ and R¹² are defined as in formula I herein; R² and R^{2′} independently are H, hydroxy, substituted or unsubstituted alkyl, alkenyl, alkynyl, or alkoxy, or are —OCOR²⁵; or R² and R^{2′} together with the carbon atom they are bonded to form a ketal, or CR²R^{2′} is C═CR²³R²⁴; R^{3′} is H and R³ is hydroxy or —OR³¹; and R⁴ and R^{4′} are H.

In another aspect, the synthetic method comprises employing at least one step comprising a site specific halogenation-dehydrohalogenation or hydroxylation of steroid derivatives, wherein, preferably, a 3-substituent is utilized to selectively provide Δ-9,11 ene or Δ-9,11-ene-12-hydroxy steroids. In one embodiment, the compound employed is of formula:

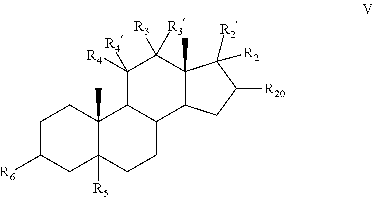

wherein R² and R^{2′} independently are H, hydroxy, substituted or unsubstituted alkyl, alkenyl, alkynyl, or alkoxy, —COR²², —OCOR²²; or R² and R^{2′} together with the carbon atom they are bonded to form a ketal, or CR²R^{2′} is oxo or C═CR²³R²⁴; R²², R²³, and R²⁴ are defined as in any aspect and embodiment herein;

R²⁰ is H or R²⁰ and R² together with the carbon atom they are bonded to form an epoxide or a double bond;

R³, R^{3′}, R⁴, and R^{4′} are H;

R⁵ is beta H;

R⁶ is —Z¹—Z²—Z³—Z⁴;

Z¹ is O, S, N(R¹⁴)₂, N(R¹⁴)₃(+), or SO₃(−);

Z² is Si(R¹⁵)₂, (CO), —SO₂—, or a bond;

Z³ is substituted or unsubstituted methylene or a bond; and

Z⁴ is aryl or substituted aryl containing one or more iodo or ICl₂ groups, or is substituted or unsubstituted heteroaryl containing at least an —N=moiety, or a heterocycle containing at least one —S— atom in the cycle; (+)N(R¹⁴)₃-aryl, (+)N(R¹⁴)₃-substituted aryl or is (−)O₃S-substituted aryl where the substituted aryl contains, among other substituents, one or more iodine atoms; provided that when Z¹ is N(R¹⁴)₃(+), Z² and Z³ are each a bond, and Z⁴ is (−)O₃S-substituted aryl, and when Z¹ is SO₃(−), Z² and Z³ are each a bond, and Z⁴ is (+)N(R¹⁴)₃-aryl or (+)N(R¹⁴)₃-substituted aryl; each R¹⁴ is alkyl; and each R¹⁵ independently is alkyl, aryl, or is a steroid, as disclosed herein, attached to the Si atom via the 3-O atom. In another embodiment, R⁶ is —O—CO—Z⁴, wherein Z⁴ is aryl or substituted aryl containing one or more iodo or ICl₂ groups, or is substituted or unsubstituted heteroaryl containing at least an —N=moiety, or a heterocycle containing at least one —S— atom in the cycle. In another embodiment, Z⁴ is aryl or substituted aryl containing one or more iodo or ICl₂ groups. In another embodiment, Z⁴ is aryl or substituted aryl containing one or more iodo groups. In another embodiment, Z⁴ is phenyl or substituted phenyl containing one or more, preferably one, iodo groups.

In one embodiment, the method comprises contacting the compound of formula V, with a halogenating agent, under a halogenation-dehydrohalogenation conditions to provide a compound of formula:

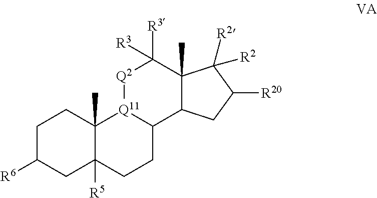

wherein Q¹¹-Q² is C═CH or is CX¹—CH (i.e., Q¹¹ is CX¹) where X¹ is halo, preferably, chloro, and the other substituents are defined as in formula V above. In another embodiment, the method comprises contacting a compound of formula VA wherein Q¹¹-Q² is CX¹—CH under dehydrohalogenation conditions to provide a compound of formula VA wherein Q¹¹-Q₂ is C═CH. In another embodiment, X¹ is chloro.

In another embodiment, the method comprises converting the compound of formula VA, wherein Z¹ is O, and Z², Z³, Z⁴, R², R^{2′}, R³, and R^{3′} are defined as in formula VA above, to a compound of formula VB

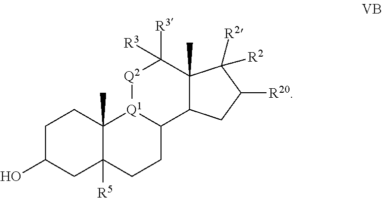

In another embodiment, the method further comprises converting compound VB via a plurality of steps to a compound of formula VC:

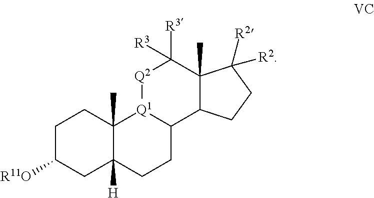

In another embodiment, the compound of formula VA, wherein Q¹¹-Q² is C═CH, is reacted with an oxidizing agent for providing a compound of formula VB, wherein CR³R^{3′} is oxo, or R^{3′} is H and R³ is hydroxy or —OOR³², and R³² is H or alkyl.

In another embodiment, provided is a method comprising converting a compound of formula VA, wherein Q¹-Q² is C═CH and CR³R^{3′} is oxo to a compound of formula VA, wherein Q¹-Q² is CH—CH₂, R^{3′} is H, and R³ is an alpha hydroxy.

In another embodiment, provided is a method comprising converting a compound of formula VA, wherein R⁶ is —Z¹—Z²—Z³—Z⁴, Z¹ is O, and Z², Z³, Z⁴ are defined as in formula V above, Q¹-Q² is CH—CH₂, R^{3′} is H, and R³ is an alpha hydroxy or CR³R^{3′} is oxo to a compound of formula VA, wherein R¹ is hydroxy.

In another embodiment, for compounds of formula V-VC, R^{2′} is H and R² is COCH₃.

Nonlimiting examples of R⁶ groups include:

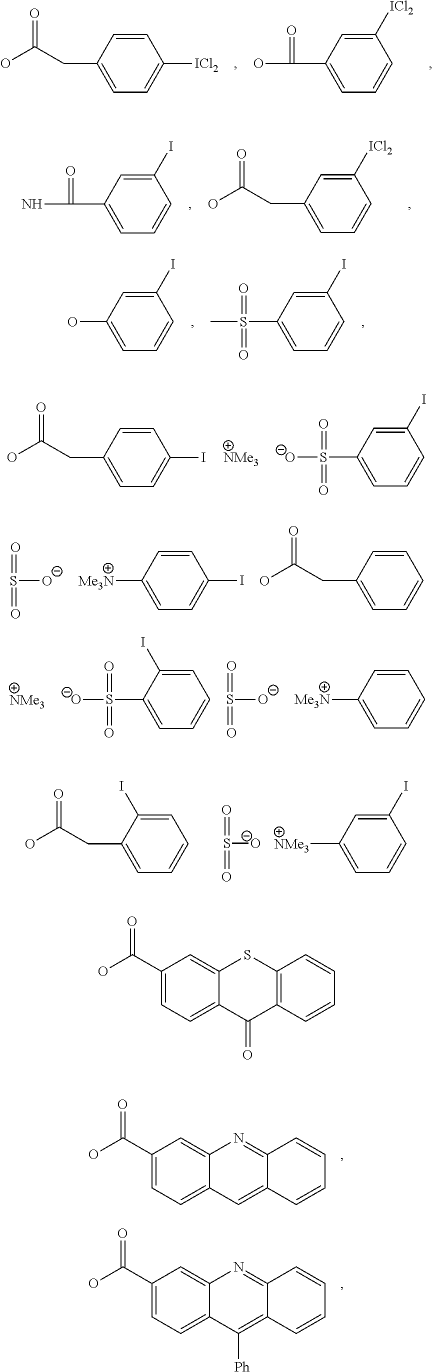

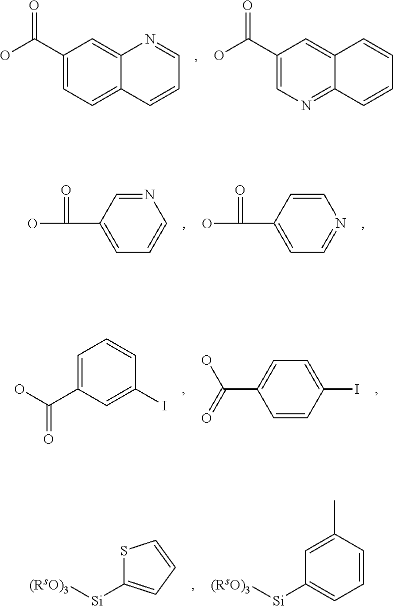

where R^{s} is a steroid moiety, joined with the O atom via its 3 position, as disclosed here.

The halogenation is carried out employing at least 1 equivalent PhICl₂ in an inert solvent under ultraviolet irradiation, for a period of time to provide a substantial amount of at least the 9-chlorinated product. Suitable solvents include dichloromethane, chloroform, and the like. The solvent is preferably free of dissolved oxygen, which can impede the reaction. The contacting is carried out at 0° C.-30° C. The dehydrohalogenation is carried out using at least 1 equivalent of a base, preferably alkali, in excess, at a temperature of 60° C.-90° C., in an inert solvent, such as dioxane, methanol, ethanol, or mixtures thereof, for a period of time to provide substantial product.

In another aspect, provided herein is a method of making a compound of formula I:

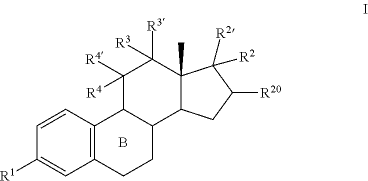

wherein R², R^{2′}, R³, R^{3′}, R⁴, and R^{4′} are defined as in any aspect and embodiment herein involving formula I. In one embodiment, R²⁰ is H. In another embodiment, CR²R^{2′} is oxo or a cyclic ketal. In another embodiment, R² and R^{2′} are H. In another embodiment, R³ is OH In another embodiment, R^{3′} is H. In another embodiment, R¹ is OR¹¹. In another embodiment, R¹¹ is H or alkyl.

Illustrative and nonlimiting embodiments are disclosed below. In one embodiment, provided is a method of making aromatic steroids, particularly, equilenin derivatives, comprising contacting a compound of formula:

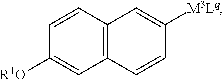

wherein M³ is a metal selected from copper, magnesium, lithium, L is an anion or a neutral ligand, q is 1-3;
with a compound of formula:

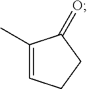

and with a compound of formula:

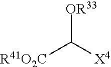

wherein X⁴ is a leaving group and R⁴¹ is substituted or unsubstituted alkyl, under conditions to form the compound of formula VIA:

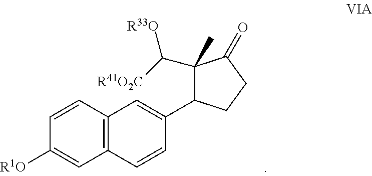

As will be apparent to the skilled artisan, tandem Michael addition (to the enone)-nucleophilic substitution (alkylation) conditions well known to the skilled artisan are employed to perform this reaction.

In one embodiment, the method further comprises contacting the compound of formula VIA with an alcohol or a diol under ketalization conditions to form the oxo protected compound (oxo protection represented by CR²R^{2′}) of formula VIB:

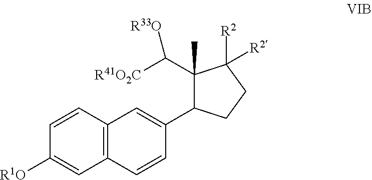

wherein R² and R^{2′} are —O—R²⁵ or CR²R^{2′} is a cyclic ketal.

In another embodiment, provided is a method comprising contacting a compound of formula VIB, wherein R⁴¹ is H, under Friedel Crafts acylation conditions to provide the compound of formula VIC

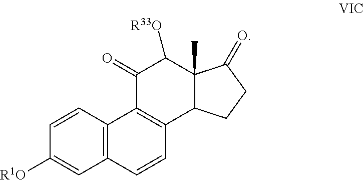

As will be apparent to the skilled artisan, Friedel Crafts acylation conditions refer to conditions under which a R^{z}—CO(+) cation is formed, where R^{z} is substituted or unsubstituted alkyl or aryl, e.g., from R^{z}—CO-L¹, where L¹ is halo, or R^{z}—CO₂H. Nonlimiting examples of reagents useful for forming R^{z}—CO(+) cations include, aluminum halides, lanthalide metal triflates, HF, and the like.

In another embodiment, the method further comprises ketalizing the compound of formula VIC to provide a compound of formula VID:

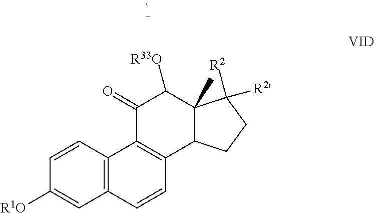

wherein CR²R^{2′} is a cyclic ketal. AS used herein, ketalizing refers to forming a cyclic or acyclic ketal from an oxo group.

In another embodiment, the method further comprises reducing the compound of formula VID to provide the compound of formula VIE or VIF:

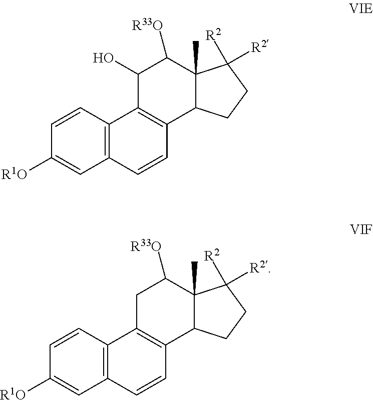

The reducing is performed using hydrogen and a hydrogenation catalyst or borohydride or aluminum hydride as reducing agents, in an inert solvent. Suitable reaction conditions for carrying out these transformations are well known the skilled artisan.

In another embodiment, the method further comprises reducing the compound of formula VIE to provide an equilenin derivative of formula VID:

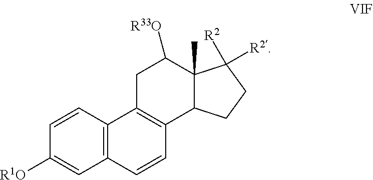

Compound VIF is conveniently converted to DCA or an intermediate thereto following methods provided herein and those known to the skilled artisan. Some illustrative steps involved in such transformations include, Birch reduction of the A, B aromatic ring, angular methylation at the 10 position, creating a cis A, B ring junction (see, e.g., U.S. 2010/0160276, supra), and elaboration of the 17-side chain following olefination and metathesis reactions.

Also provided herein are methods for making cholic acid for example as shown below:

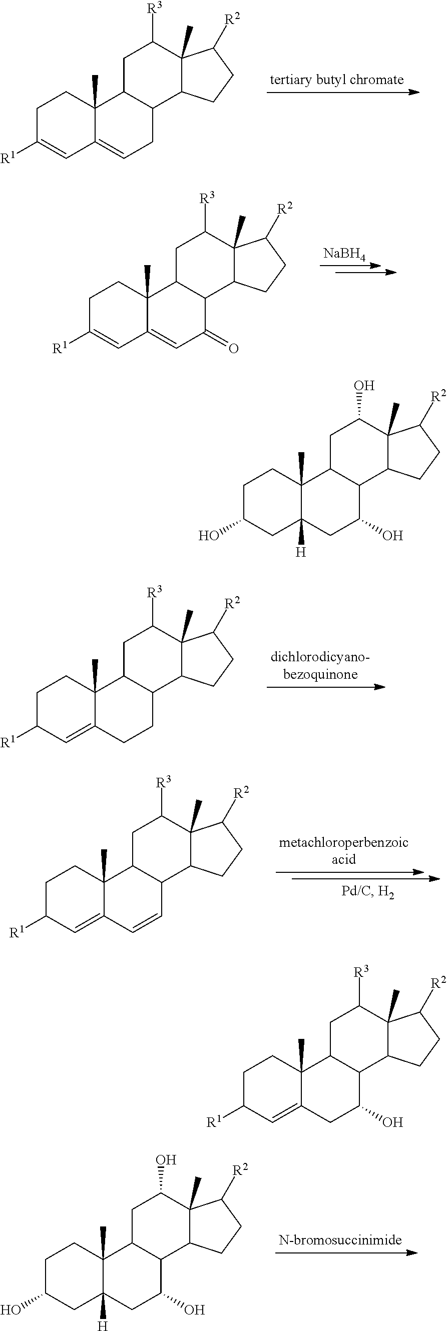

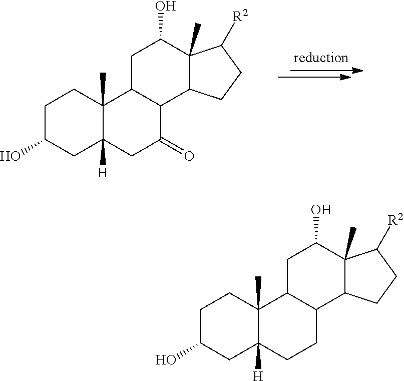

Cholic acid, i.e., when R² is:

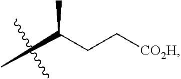

and the steroid scaffold contains 3-alpha, 7-alpha, and 12-alpha hydroxy groups, or a salt or carboxyl ester thereof, is conveniently converted to DCA, e.g., by selectively oxidizing the 7-OH group to a 7-oxo group and reducing the 7-oxo group to a methylene moiety.

In some embodiments, provided herein are methods for resolving enantiomeric (i.e., 50:50 mixture of R and S enantiomers) or scalemic (i.e., mixtures of unequal amounts of enantiomers) mixtures of DCA or an intermediate thereto. In certain instances, the synthetic methods employ steroids that would be in one enantiomeric form, chemical modifications of which yields diastereomers that would be separated by chromatography.

In another aspect, the synthetic bile acids of this invention are represented by formula VII:

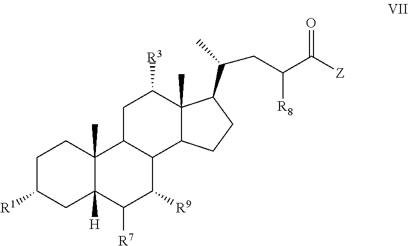

wherein:
R⁷ is hydrogen, halo, alkyl, alkenyl, alkynyl, or alkoxy;
R⁸ is hydrogen, halo, alkyl, alkenyl, alkynyl, alkoxy, or haloalkyl;
R¹, R³, and R⁹ are each independently hydrogen, hydroxy, or alkoxy;
Z is hydroxy, alkoxy, —NH₂, or

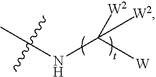

where t is 1 or 2, w¹ and w² are each independently H or (C₁₋₄)alkyl optionally substituted with hydroxy, alkoxy, thio, thioalkyl, amino, substituted amino, aryl, and substituted aryl, and W is —COOH or —SO₃H; or a salt thereof;
provided that when R⁷ and R⁸ are hydrogen and R⁹ and Z are hydroxy, then R³ is not hydroxy.

In one embodiment, the C¹⁴ content of the synthetic bile acids of this invention are different than those of naturally occurring bile acids. In some embodiments, the C¹⁴ content of the bile acids of this invention are less than 1 ppt.

In one embodiment, R⁷ and R⁸ are hydrogen and R¹, and R³, R⁹ are hydroxy. In one embodiment, R⁷ is hydrogen and R¹, R³, R⁹, and Z are hydroxy.

In another embodiment, R¹, R⁷, and R⁸ and are hydrogen and R⁹ and Z are hydroxy.

In another embodiment, R³, R⁷, R⁸, and R⁹ are hydrogen and Z is hydroxy.

In another embodiment, R⁷ and R⁸ is hydrogen, R¹, R³, and R⁹ are hydroxy, and Z is —NHCH₂COOH or —NHCH₂CH₂SO₃H.

In still another embodiment, R⁷ is C₁-C₄ alkyl, and R¹, R³, R⁹, and Z are hydroxy.

In one of its composition aspects, this invention is directed to a composition comprising an inert diluent and a compound of formula VII above. In a preferred embodiment, the composition is a pharmaceutically acceptable composition and the diluent is a pharmaceutically acceptable carrier.

This invention is also directed to methods for preparing compounds of formula VII above.

### DETAILED DESCRIPTION OF THE INVENTION

### Definitions

This invention is directed to the preparation of bile acids, such as deoxycholic acid, cholic acid, chenodeoxycholic acid, lithocholic acid, their amino acid conjugates, and methods of use thereof. Accordingly, the C ring of a steroidal scaffold, preferably that of an aromatic or an A,B-trans steroid, is oxidized to provide synthetic routes and intermediates to bile acids. Thus, e.g., this invention provides synthetic methods for preparing a bile acid or a salt thereof starting from aromatic steroids such as estrogen, equilenin, equilin and derivatives thereof. This invention is also directed to intermediates such as 12-oxo or delta-9,11-ene steroids as well as novel processes for their preparation. In preferred embodiments, bile acids are provided herein which have substituents on the B-ring and/or D-ring side chain and optionally on the hydroxy group of the A-ring. However, prior to describing this invention in greater detail, the following terms will first be defined.

It is also to be understood that the terminology used herein is for the purpose of describing particular embodiments only, and is not intended to be limiting, since the scope of the present invention will be limited only by the appended claims.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

As used herein, certain terms may have the following defined meanings. As used in the specification and claims, the singular form “a,” “an” and “the” include singular and plural references unless the context clearly dictates otherwise. Thus, for example, reference to “a solvent” includes a plurality of the same or different solvents.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations. Each numerical parameter should at least be construed in light of the number of reported significant digits and by applying ordinary rounding techniques.

The numbering of the steroidal scaffold and the rings in it, as used herein, follows the general convention:

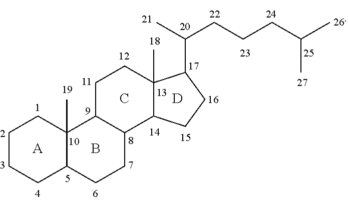

As used herein, even without specific designation, the stereochemistry at the B, C, D ring junctions is that most commonly found in natural steroids, i.e.:

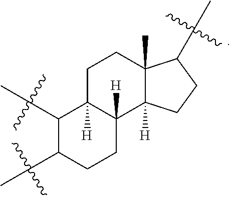

At the 3, 5, and 20-positions, the compounds includes all epimers at these positions.

It is to be understood that unless otherwise specified, the scaffolds only represents the position of carbon atoms. One or more bonds between two adjacent carbon atoms may be a double bond and one or more of carbon atoms be may optionally substituted.

The term “Δ (or delta)-9,11-ene steroidal” or “Δ-9,11-ene compound” as used herein refers to a steroidal compound having a double bond between the 9 and 11 carbon atoms which is represented by the scaffold of:

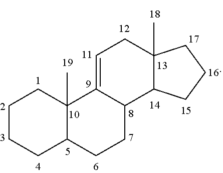 [SMILES: CC12CC=C3C(CCC4CCCCC34C)C1CCC2]

The term “12-hydroxy steroid” or “12-hydroxy compound” and synonyms thereof as used herein refers to a steroidal compound having a hydroxy substituent on the 12-position carbon atom.

The term “12-oxo steroidal” or “12-oxo compound” as used herein refers to a steroidal compound having a oxo substituent on the 12-position carbon atom which is represented by the scaffold of:

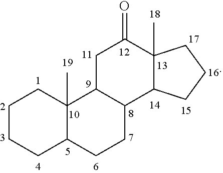 [SMILES: CC12CCCC1C1CCC3CCCCC3(C)C1CC2=O]

The term “about” when used before a numerical designation, e.g., temperature, time, amount, and concentration, including range, indicates approximations which may vary by (+) or (−) 10%, 5% or 1%.

The term “acid” refers to regents capable of donating H⁺ or to “Lewis acids” that are electron pair acceptors. Lewis acids include oraganometallic reagents such as alkyl aluminum halides (e.g. Et₂AlCl and MeAlCl₂).

The term “acylal” refers to a group having two —O(C═O)R^{k} groups attached to the same carbon atom in a molecule, where R^{k} represents an alkyl group or the two R^{k} groups together with the carbon atom and the two —O(C═O)— groups attached thereto form a ring structure. The two —O(C═O)R^{k} groups may be the same or different.

The term “acetylating reagent” refers to a reagent in which can add an acetyl (Ac) group CH₃C(O)— to a hydroxy moiety of a molecule.

The term “alkyl” refers to monovalent saturated aliphatic hydrocarbyl groups having from 1 to 10 carbon atoms (i.e., C₁-C₁₀ alkyl) or 1 to 6 carbon atoms (i.e., C₁-C₆ alkyl), or 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). The term “substituted alkyl” refers to an alkyl group where 1-5 hydrogens are substituted independently with halo, vinyl, ethynyl, phenyl or substituted phenyl, hydroxy, amino, —CO₂H, trialkylsilyl, —O-alkyl, or acetoxy group.

The term “alkenyl” refers to monovalent aliphatic hydrocarbyl groups having from 1 to 10 carbon atoms or 1 to 6 carbon atoms and 1 or more, preferably 1, carbon carbon double bond. Examples of alkenyl include vinyl, allyl, dimethyl allyl, and the like. The term “substituted alkenyl” refers to an alkenyl group where 1-5 hydrogens are substituted independently with halo, phenyl or substituted phenyl, hydroxy, amino, —CO₂H, —O-alkyl, or acetoxy, group.

The term “alkoxy” refers to —O-alkyl, where alkyl is as defined above. “Substituted alkoxy” refers to —O-substituted alkyl.

The term “alkynyl” refers to monovalent aliphatic hydrocarbyl groups having from 1 to 10 carbon atoms or 1 to 6 carbon atoms and 1 or more, preferably 1, carbon carbon triple bond. Examples of alkenyl include ethynyl, propargyl, dimethylpropargyl, and the like. The term “substituted alkynyl” refers to an alkynyl group where 1-5 hydrogens are substituted independently with halo, phenyl or substituted phenyl, hydroxy, amino, —CO₂H, —O-alkyl, or acetoxy, group.

The term “allylic oxidation” refers to oxidizing the alpha position of a double bond, preferably by incorporating one or more of a hydroxy, —OOH, —OO-alkyl, and oxo group at that alpha position.

The term “amino” refers to —NH₂. The term “substituted amino” refers to —NHR^{a} or —N(R^{a})₂ wherein Rₐ is substituted or unsubstituted, alkyl, aryl, cycloalkyl, heteroaryl, or heterocyclyl, or N(R^{a})₂ is a ring system.

The term “aryl” refers to a monovalent, aromatic ring having 6-10 ring carbon atoms. Examples of aryl include phenyl and napthyl. The term “substituted aryl” refers to an aryl group where 1-5 hydrogens are substituted independently with halo, vinyl, ethynyl, phenyl, hydroxy, amino, —CO₂H, —O-alkyl, or acetoxy, group.

The term “bile acid” refers to a large family of molecules, composed of a steroid structure with four rings, a five or eight carbon side-chain terminating in a carboxylic acid joined at the 17-position of the steroid scaffold, and the presence and orientation of different numbers of hydroxy groups. Certain bile acids for use in the methods disclosed herein include those shown in Scheme 1.

The term “chromium oxidizing agents” refers to hypervalent chromium compounds, e.g., chromium VI compounds capable of effecting oxidation. In one embodiment, the chromium oxidizing agent is capable of oxidizing primary alcohols to aldehydes and secondary alcohols to ketones. Such selective chromium oxidizing agents are typically complexed with a base such as pyridine. One particularly preferred chromium oxidizing agent is pyridinium chlorochromate. In another embodiment, the chromium oxidizing agent is capable of oxidizing a methylene group alpha to vinyl unsaturation to effect formation of an allylic ketone. In that embodiment, preferred chromium oxidizing agents include chromium trioxide and a co-oxidant mixture of NaOCl and t-alkyl hydrogen peroxide such as t-butyl hydrogen peroxide (TBHP).

As used herein, the term “comprising” is intended to mean that the compounds and methods include the recited elements, but not excluding others. “Consisting essentially of” when used to define compositions and methods, shall mean excluding other elements of any essential significance to the compounds or method. “Consisting of” shall mean excluding more than trace elements of other ingredients for claimed compounds and substantial method steps. Embodiments defined by each of these transition terms are within the scope of this invention. Accordingly, it is intended that the methods and compounds can include additional steps and components (comprising) or alternatively include additional steps and compounds of no significance (consisting essentially of) or alternatively, intending only the stated methods steps or compounds (consisting of).

The term “copper oxidizing agents” refer to copper compounds capable of effecting oxidation.

The term “cycloalkyl” refers to a monovalent, preferably saturated, hydrocarbyl ring having 6-10 ring carbon atoms. Nonlimiting examples of cycloalkyl include cyclopropyl, cyclobutyl, cyclopentyl, cyclohexyl, adamentyl, and the like. The term “substituted cycloalkyl” refers to a cycloalkyl group where 1-5 hydrogens are substituted independently with halo, vinyl, ethynyl, phenyl, hydroxy, amino, —CO₂H, —O-alkyl, or acetoxy group.

The term “dehydration reagent” refers to a reagent that can react with a hydroxy group, and chemically remove water (H₂O) from a molecule.

The term “elimination conditions” refers to reaction conditions in which a small molecule, such as H₂O, HCl, or HBr, HI, etc., is eliminated from a compound comprising a hydroxy, chloro, bromo, or iodo group, etc. to form a corresponding compound comprising a carbon carbon double bond. In one example, an elimination condition includes dehydration conditions wherein the hydroxy group and the vicinal hydrogen atom are eliminated to form a vinyl group (an “ene”) group. Dehydration conditions may include converting the hydroxy group to a leaving group such as chloro, bromo, tosylate, mesylate, triflate, or —OS(O)Cl. Such dehydration or dehydrating is accomplished, for example by a dehydration reagent or simply by heating. In another example, an elimination condition includes dehydrohalogenation conditions wherein the halo atom and the vicinal hydrogen atom are eliminated to form a vinyl group (an “ene”) group.

The term “haloalkyl” refers to monovalent saturated aliphatic hydrocarbyl groups having from 1 to 10 carbon atoms and from one to three halo atoms (i.e., F, Cl, Br or I).

The term “heteroaryl” refers to a monovalent, hydrocarbyl, aromatic ring having 6-14 ring carbon atoms and 1-6 heteroatoms selected preferably from N, O, S, and P. Nonlimiting examples of heteroaryl include imidazole, pyridine, quinoline, and the like. The term “substituted heteroaryl” refers to a heteroaryl group where 1-5 hydrogens are substituted independently with halo, vinyl, ethynyl, phenyl, hydroxy, amino, —CO₂H, —O-alkyl, or acetoxy group.

The term “heterocycle” refers to a monovalent, nonaromatic, ring having 6-10 ring carbon atoms and 1-6 heteroatoms selected preferably from N, O, S, and P. Nonlimiting examples of cycloalkyl include pyrrolidinyl, piperidinyl, piperizinyl, and the like. The term “substituted heterocycle” refers to an aryl group where 1-5 hydrogens are substituted independently with halo, vinyl, ethynyl, phenyl, hydroxy, amino, —CO₂H, —O-alkyl, or acetoxy group.

The term “hydroxy protecting group” refers to a group capable of protecting the hydroxy (—OH) group of a compound and releasing the hydroxy group under deprotection conditions. Common such groups include acyl (which forms an ester with the oxygen atom of the hydroxy group), such as acetyl, benzoyl, and groups that form an ether with the oxygen atom of the hydroxy group, such as methyl, allyl, propargyl, benzyl, methoxybenzyl, and methoxymethyl, silyl ethers, etc. Hydroxy protecting groups are well known in the field of organic synthesis.

The term “hydrogenation conditions” refers to conditions and catalysts for introducing H₂ across one or more double bonds, preferably using a hydrogenation catalyst. Hydrogenation catalysts include those based on platinum group metals (platinum, palladium, rhodium, and ruthenium and their oxides and hydroxides) such as Pd/C and PtO₂.

The term “ketal” refers to a group having two —OR^{x} groups attached to the same carbon atom in a molecule, where R^{x} represents an alkyl group, or the two R^{x} groups together with the carbon atom and the two oxygen atoms attached thereto form a ring structure (also referred to here as a cyclic ketal). The two —OR^{x} groups may be the same or different. Nonlimiting examples of cyclic ketals include:

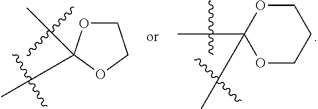 [SMILES: *C1(*)OCCO1 ; *C1(*)OCCCO1]

The term “olefination reagent” refers to regents that perform olefination, i.e., react with ketones to form olefins. The term “olefin forming conditions” refers to conditions to carry out such transformations. Examples of such reagents include Wittig and Wittig Horner reagents and examples of such conditions include Wittig and Wittig Horner olefination conditions.

The term “oxidizing” with respect to a molecule refers to removing electrons from that molecule. In this way, for example, oxygen can be added to a molecule or hydrogen can be removed from a molecule. Oxidizing is effected, e.g., by oxidizing agents and by electrochemically. The term “oxidizing conditions” refers to suitable conditions for oxidizing a molecule including microbial oxidation as disclosed herein.

The term “oxidizing agent” refers to a reagent which is capable of oxidizing a molecule, and include, without limitation, “chromium oxidizing agents” and “copper oxidizing agents”. In this way, oxygen can be added to a molecule or hydrogen can be removed from a molecule. In one example, the oxidizing agent oxidizes vicinal (1,2) alcohols and includes periodate compounds. Such oxidizing agents are sometimes referred to as “vicinal alcohol oxidizing agents”. Oxidizing agents include by way of example only dioxirane, ozone, di-^{t}butyltrioxide, oxygen, chloranil, dichlorodicyanobezoquinone, peracids, such as percarboxylic acids, Jones reagent, alkyl hydroperoxides, such as tertiary-butyl hydroperoxide (optionally used with CuI and a hypochlorite), hypochlorite, pyridinium chlorochromate, CrO₃, and Cu (II) or Cu (III) compounds, or mixtures thereof. More than one oxidizing agents may be used together for oxidizing a compound, where one of the oxidizing agents, preferably the metal-containing oxidizing agent, such as a chromium or a copper oxidizing agent, may used in a catalytic amount.

The term “oxo” or keto refers to the group (>C═O).

The term “oxo protecting group” refers to a group capable of protecting a oxo group of a compound and releasing the oxo group under deprotection conditions. Common such groups include ketals, cyclic ketals, and acylals. Oxo protecting groups are well known in the field of organic synthesis. Suitable hydroxy or oxo protecting groups and other protecting groups which may be employed according to this invention, and the conditions for their removal, are described in books such as Protective groups in organic synthesis, 3 ed., T. W. Greene and P. G. M. Wuts, eds., John Wiley & Sons, Inc., New York, N.Y., U.S.A., 1999, and will be well known to a person of ordinary skill in the art, which is incorporated by reference in its entirety.

The term “pharmaceutically acceptable salt” refers to nontoxic pharmaceutically acceptable salts derived from a variety of organic and inorganic counter ions well known in the art and include, by way of example only, sodium, potassium, calcium, magnesium, ammonium, and tetraalkyl ammonium. When the active agent contains a basic functionality, pharmaceutically acceptable salts include, by way of example only, chloride, bromide, sulfate, phosphate, various carboxylates and various sulfonates.

The term “reducing” refers to addition of one or more electrons to a molecule, and for example, allowing hydrogen to be added to a molecule and include hydrogenation conditions. The term “reducing agent” refers to a reagent which can donate electrons in an oxidation-reduction reaction, and, for example, allowing hydrogen to be added to a molecule. The term “reducing conditions” refers to suitable conditions, including hydrogenation conditions, for allowing electron and/or hydrogen to be added to a molecule. Suitable reducing agents include, without limitation, lithium, sodium, potassium, aluminum amalgam, lithium aluminum hydride, sodium borohydride, sodium cyanoborohydride, lithium tri-^{t}butoxy aluminum hydride, di^{t}butoxy aluminum hydride, lithium triethyl borohydride and the like.

As used herein, for example, “substituted or unsubstituted alkyl, alkenyl, or alkynyl” refers to substituted or unsubstituted alkyl, substituted or unsubstituted alkenyl, or substituted or unsubstituted alkynyl.

The term “substituted phenyl” includes a phenyl group where 1-3 hydrogen atoms are substituted with methyl, t-butyl, methoxy, halo, nitro, NHCOCH₃, or NHCO₂-^{t}butyl.

The term “thio” refers to —SH.

The term “thioalkyl” refers to —S-alkyl.

### Synthetic Methods

In one aspect, this invention provides a method of synthesis comprising reducing a compound of formula:

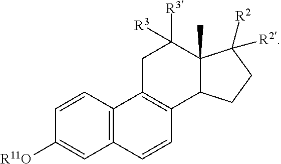

wherein R¹¹ is substituted or unsubstituted alkyl; R² and R^{2′} are independently H and OR²², provided that one of R² and R^{2′} is OR²², or CR²R^{2′} is oxo, or R² and R^{2′} together with the carbon atom they are attached form a cyclic ketal; R²² is H or substituted or unsubstituted alkyl, alkenyl, alkynyl, or aryl; R³ and R^{3′} are independently H and OR³¹, provided that one of R³ and R^{3′} is OR³¹; or CR³R^{3′} is oxo; R³¹ is H or substituted or unsubstituted alkyl or alkenyl; under a reducing conditions to provide a compound of formula:

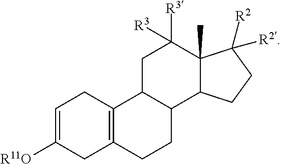

In one embodiment, the method further comprising contacting the compound of formula:

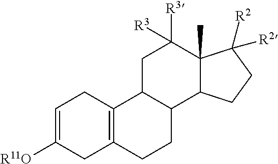

with a an alcohol or a diol under ketalization conditions to provide a compound of formula:

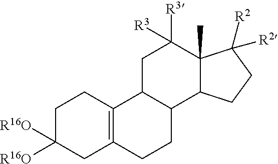

wherein R¹⁶ is substituted or unsubstituted alkyl or 2 R¹⁶ groups together with the oxygen atoms they are attached to, form a cyclic ketal, and R², R^{2′}, R³, R^{3′}, and R¹¹ defined as in the previous paragraph.

In another embodiment, the method further comprising contacting the compound of formula:

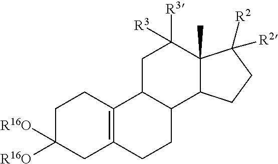

with a carbene of formula CX₂ or a precursor thereof wherein each X independently is H or halo, to provide a compound of formula:

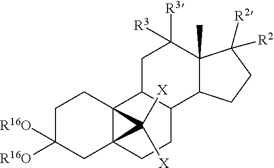

wherein R², R^{2′}, R³, R^{3′}, R¹¹ and R¹⁶ are defined as in the previous paragraph.

In another embodiment, the method further optionally comprising contacting the compound of formula:

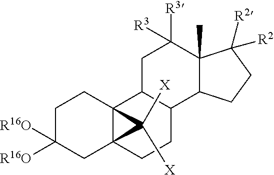

wherein at least one X is halo, with a reducing agent to provide a compound of formula:

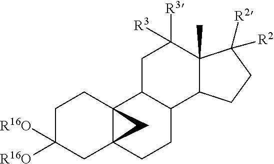

wherein R², R^{2′}, R³, R^{3′}, R¹¹ and R¹⁶ are defined as in the previous paragraph.

In another embodiment, the method further comprising contacting the compound of formula:

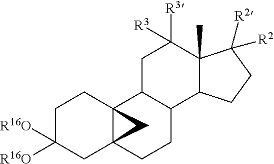

with an acid under conditions to provide a compound of formula:

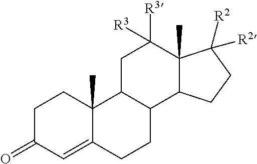

wherein R² is OR²², R^{2′} is H, or CR²R^{2′} is oxo, and R³ and R^{3′} are defined as in the previous paragraph.

In another embodiment, the compound of formula:

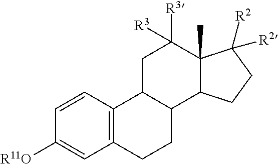

wherein R³ is hydroxy, R^{3′} is H, and CR²R^{2′} is oxo, is synthesized comprising oxidizing a compound of formula:

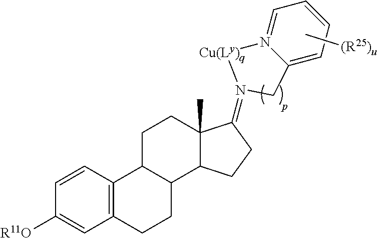

wherein p is 1 or 2, each R²⁵ independently is H or substituted or unsubstituted alkyl or aryl, L^{y} is an anion having a charge of −1 to −3, and q is 1, 2, or 3.

In another embodiment, the compound of formula:

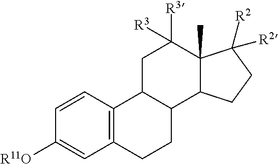

wherein R², R^{2′}, R³, R^{3′}, and R¹¹ are defined as in the previous paragraph is synthesized comprising reducing a compound of formula:

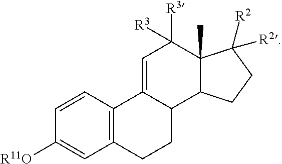

In another embodiment, the compound of formula:

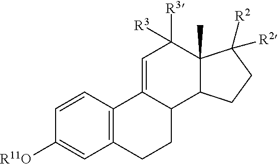

wherein R³ is OH and R^{3′} is H or CR³R^{3′} is oxo, is synthesized comprising oxidizing a compound of formula:

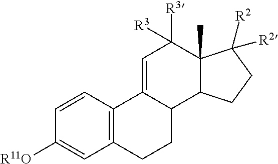

wherein R³ and R^{3′} are H.

In another embodiment, the compound of formula:

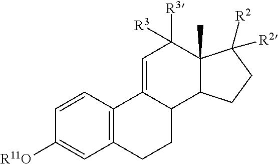

wherein R³ and R^{3′} are H is synthesized by dehydrating a compound of formula:

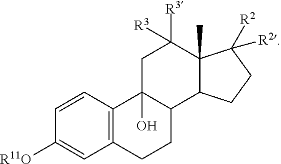

In another embodiment, the compound of formula:

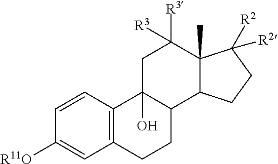

is synthesized comprising oxidizing a compound of formula:

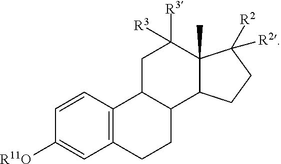

In another embodiment, provided herein is a method comprising

(i) contacting a compound of formula:

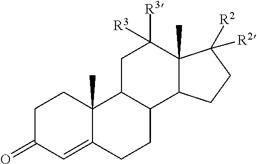

wherein R² is OR²², R^{2′} is H, or CR²R^{2′} is oxo, and R³ and R^{3′} are independently H and OR³¹, provided that one of R³ and R^{3′} is OR³¹; or CR³R^{3′} is oxo, with R¹²COL¹ wherein R¹² is substituted or unsubstituted alkyl and L¹ is halo under acylation conditions to provide a compound of formula:

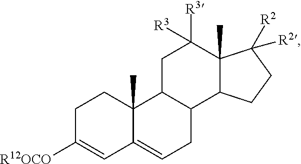

(ii) contacting the compound of formula:

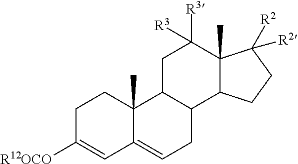

with an oxidizing agent under oxidizing conditions to provide the compound of formula:

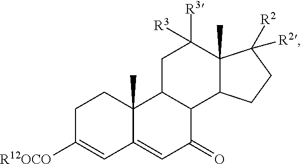

(iii) contacting the compound of formula:

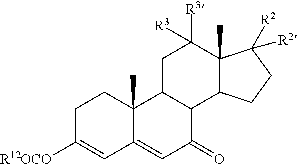

with a reducing agent under reducing conditions to provide a compound of formula:

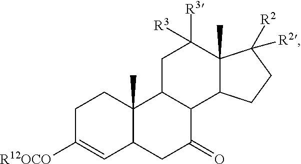

which is easily converted to cholic acid following methods disclosed here and known to the skilled artisan.

In another embodiment, provided herein is a method comprising:

(i) contacting a compound of formula:

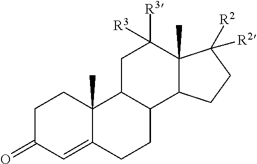

wherein R² is substituted or unsubstituted alkyl or OR²², R² is H, or CR²R^{2′} is oxo, R³ and R^{3′} are independently H, OH, and OR³¹, provided that one of R³ and R^{3′} is OR³¹ or CR³R^{3′} is oxo, and R³¹ is substituted or unsubstituted alkyl; with an oxidizing agent under oxidation conditions to provide a compound of formula:

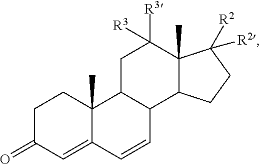

(ii) contacting the compound of formula:

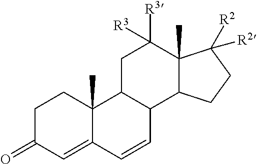

with an epoxidizing agent under oxidizing conditions to provide a compound of formula:

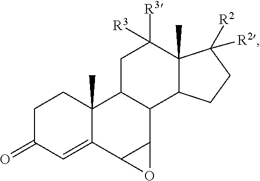

(iii) contacting the compound of formula:

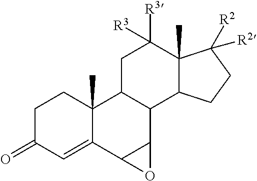

with a reducing agent under reducing conditions to provide a compound of formula:

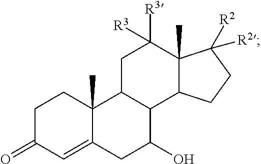

(iv) contacting the compound of formula:

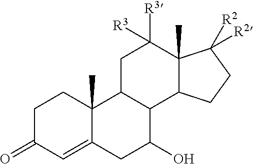

with a reducing agent under reducing conditions to provide the compound of formula:

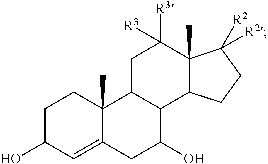

and
(v) contacting the compound of formula:

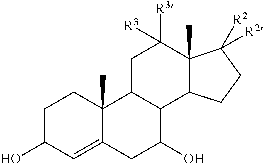

with hydrogen under hydrogenation conditions to provide the compound of formula:

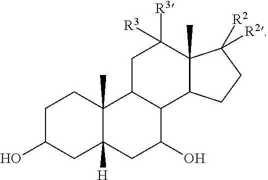

In the method above, step (i) is performed using chloranil or another quinone. A suitable epoxidizing agent is meta chloroperbenzoic acid or another percarboxylic acid or another peracid. The reduction is step (iii) is performed using a single electron transferring reducing agent such as aluminum amalgam. The oxo group at the 3 position is reduced using ditertiarybutyloxy aluminum hydride. The 3,4ene is reduced under hydrogenation conditions employing a hydrogenation catalyst such as Pd/C. These reactions are carried out in inert solvents well known to the skilled artisan. The reactions are carried out for a period of time to obtain a substantial amount of the product. In another embodiment, R³ is —OH and R^{3′} is hydrogen. In another embodiment, R² is

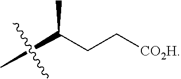

Certain preferred steps of this invention producing DCA, intermediates thereto, and certain novel compounds of this invention are schematically shown herein below.

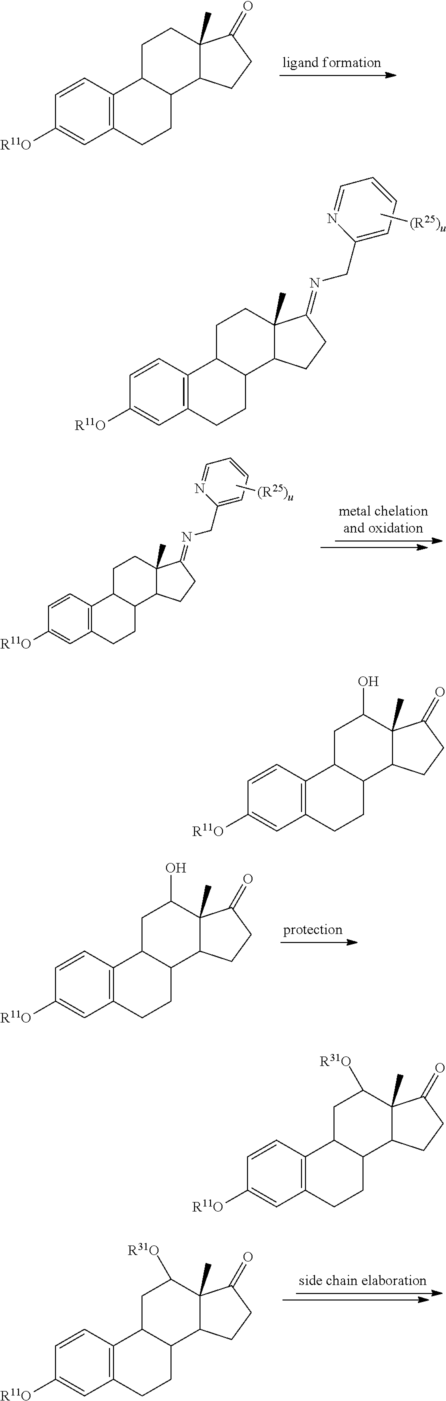

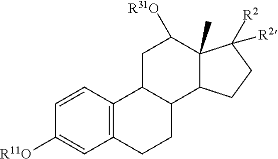

In the schemes below the conversion of a protected 12-hydroxylated estrogen derivative to DCA via novel androstene-3,17-dione intermediates is shown.

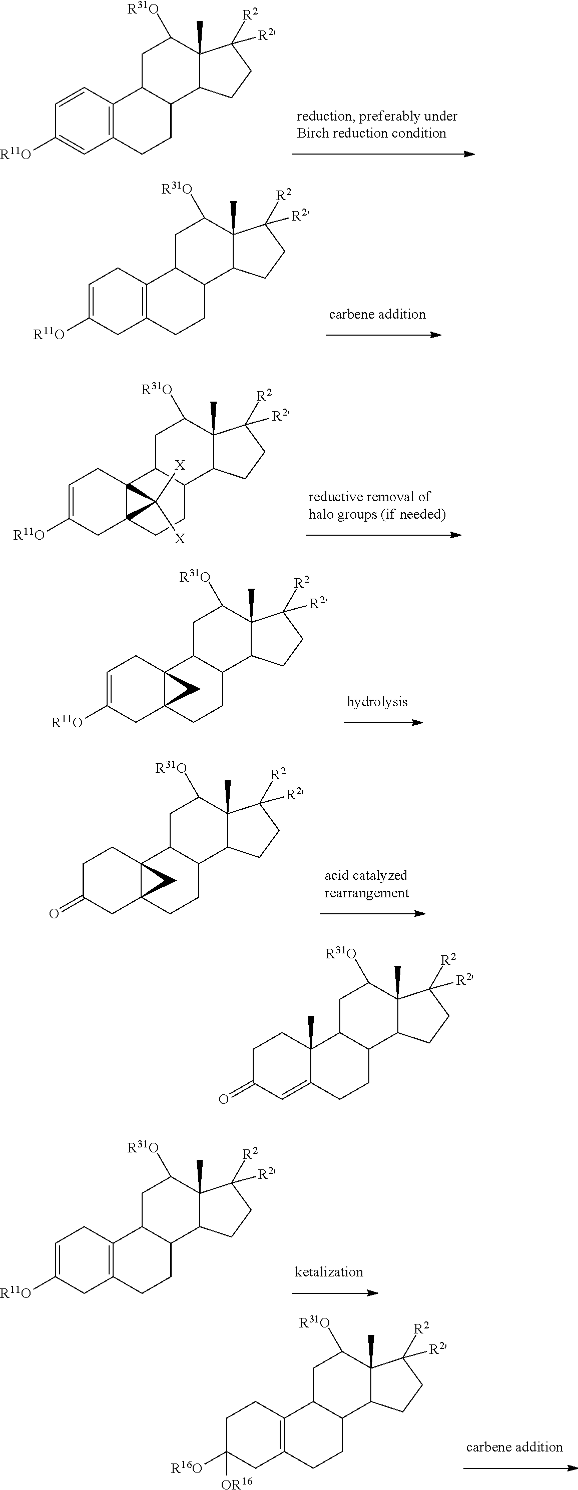

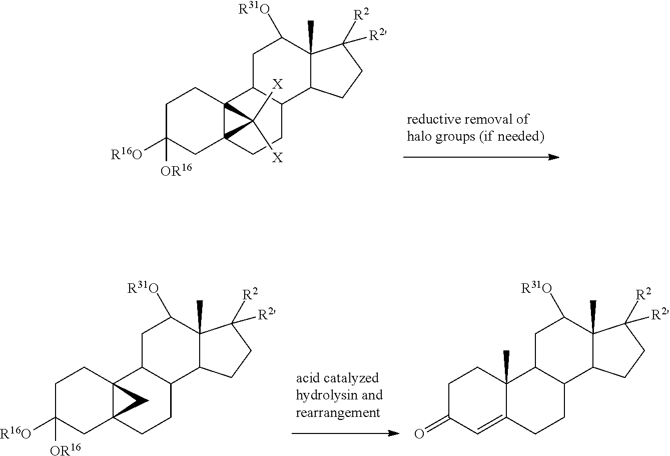

Enones, such as androstene-3,17-diones or their 17-oxo protected derivatives, containing a 12-hydroxy or a protected 12-hydroxy group, which exists preferably as the 12-beta stereoisomer or as a mixture of 12-alpha and 12-beta epimers, is converted to useful intermediates for synthesizing DCA as shown below.

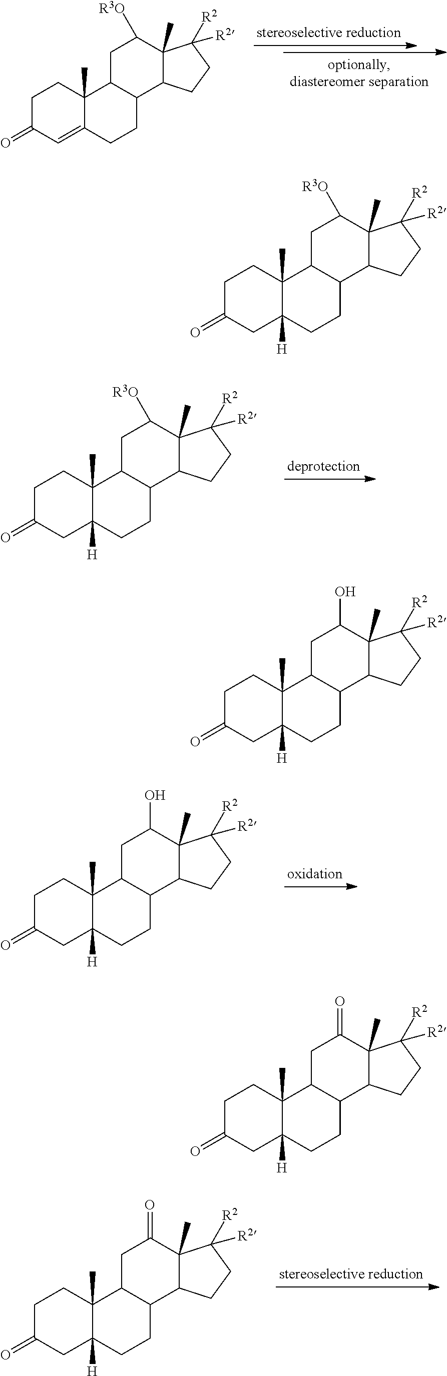

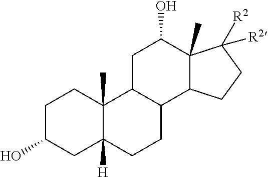

Synthesizing novel intermediates and DCA via aromatic steroidal equilenin derivatives are shown below.

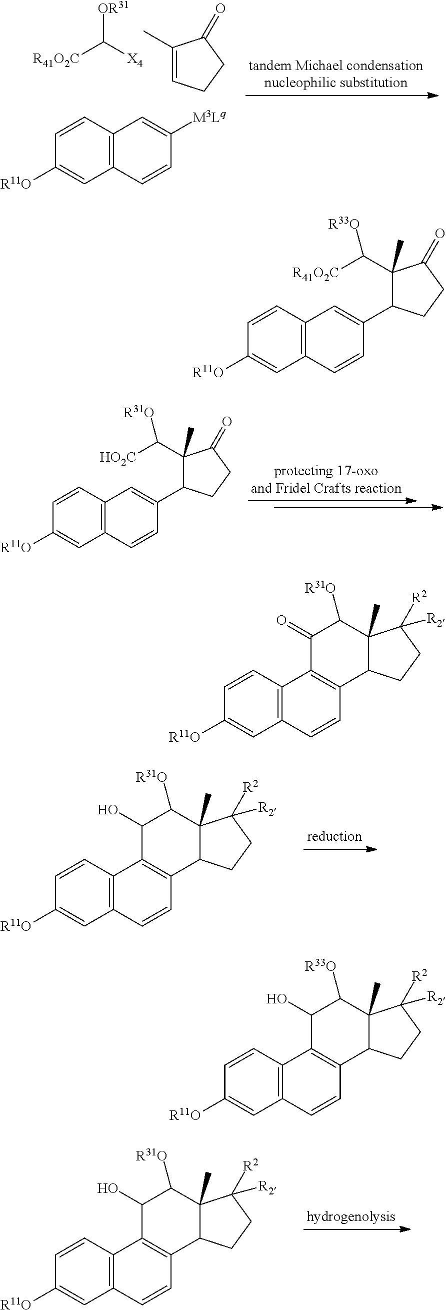

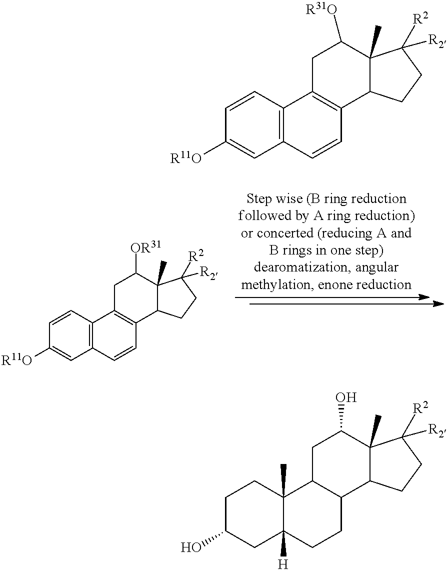

Incorporating a 12-hydroxy group on a steroid via site specific remote functionalization, preferably a halogenation, delta-9,11-ene dehydrohalogenation, en route to various novel intermediates and DCA is shown below.

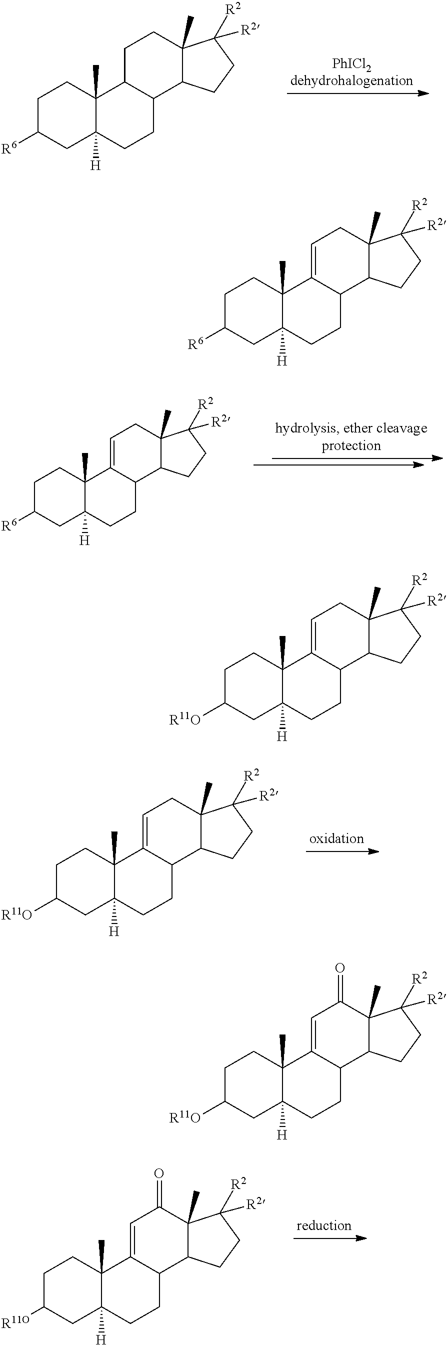

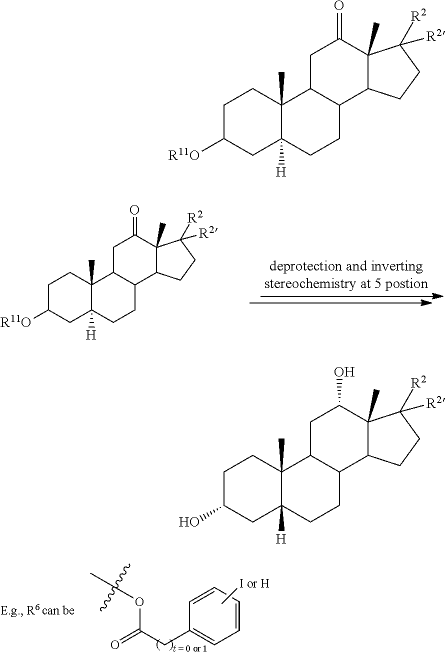

A further example of oxidation of steroids via site specific remote functionalization en route to various novel intermediates and DCA is shown below.

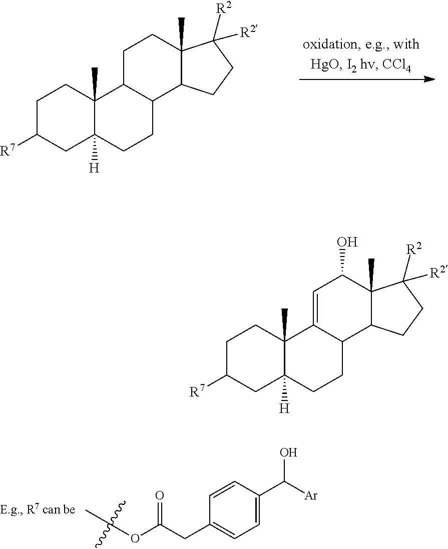

Ar is substituted or unsubstituted aryl, such as, phenyl

A method of hydroxylating the 9-position of an estrogen derivative, en route to various novel intermediates and DCA is shown below.

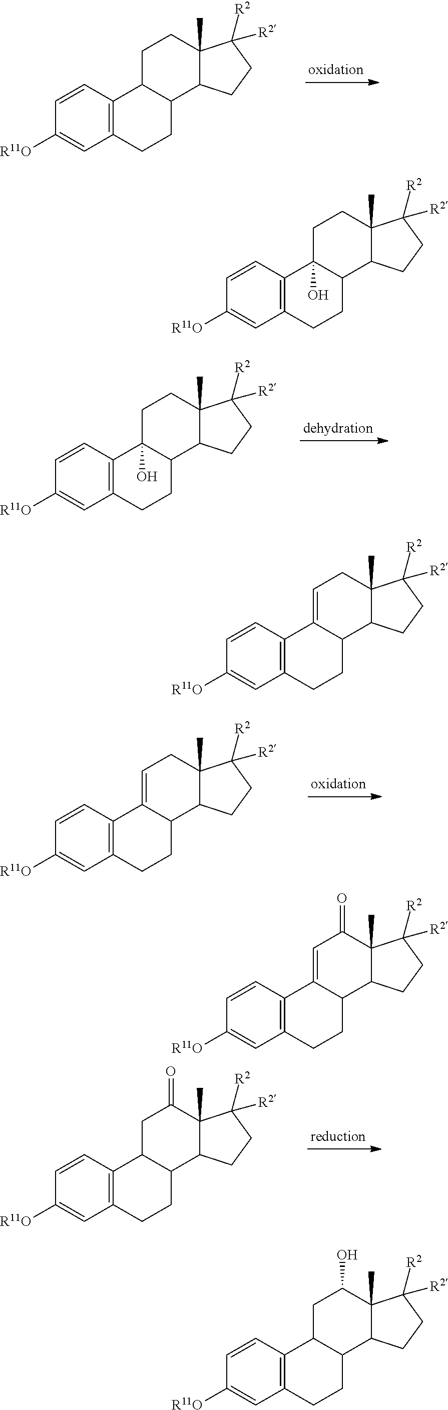

Shown below are methods for making intermediates for synthesizing DCA employing tandem ring formation while starting from compounds that are easily made.

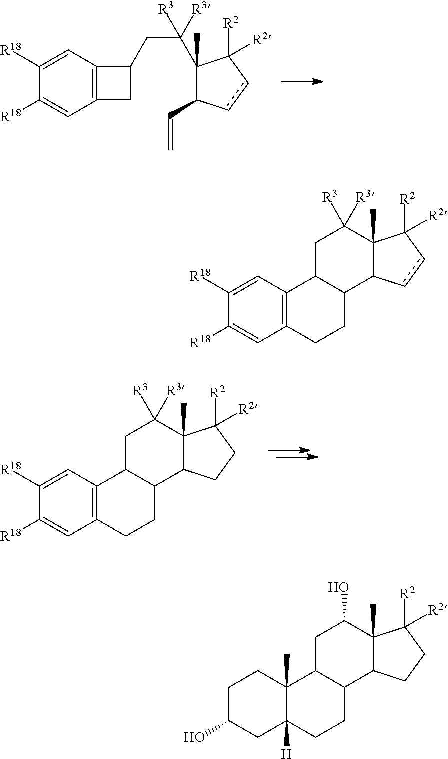

Each R¹⁸ independently is trialkylsilyl, H, or —O-alkyl. Methods for making the starting material can be adapted from the reference Funk et al., Chem. Soc. Rev., 1980, 9, 41-61, incorporated herein by reference.

In another embodiment, cascade polyene cyclization is utilized to synthesize novel intermediates for synthesizing DCA, as shown below. In this process, the generation of the A, B cis steroidal intermediate is advantageous because it avoids the A, B trans to A, B cis transformations.

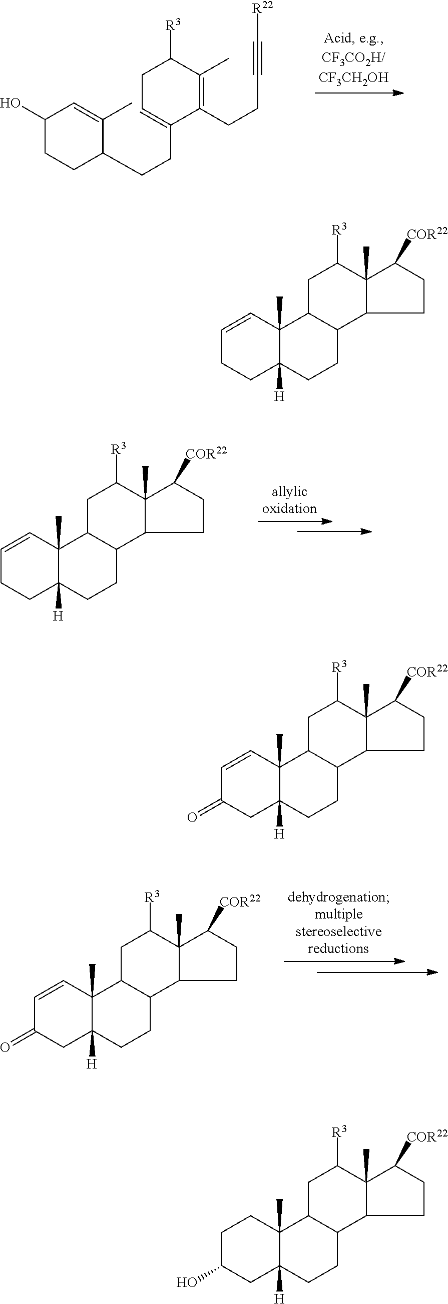

The process below provides another convenient access to DCA via 12-hydroxyprogesterone or derivatives thereof. The starting material used in the polyene cyclization may be conveniently obtained by adapting methods described in the reference Johnson, Bioorganic Chemistry, 5, 51-98 (1976), incorporated herein by reference.

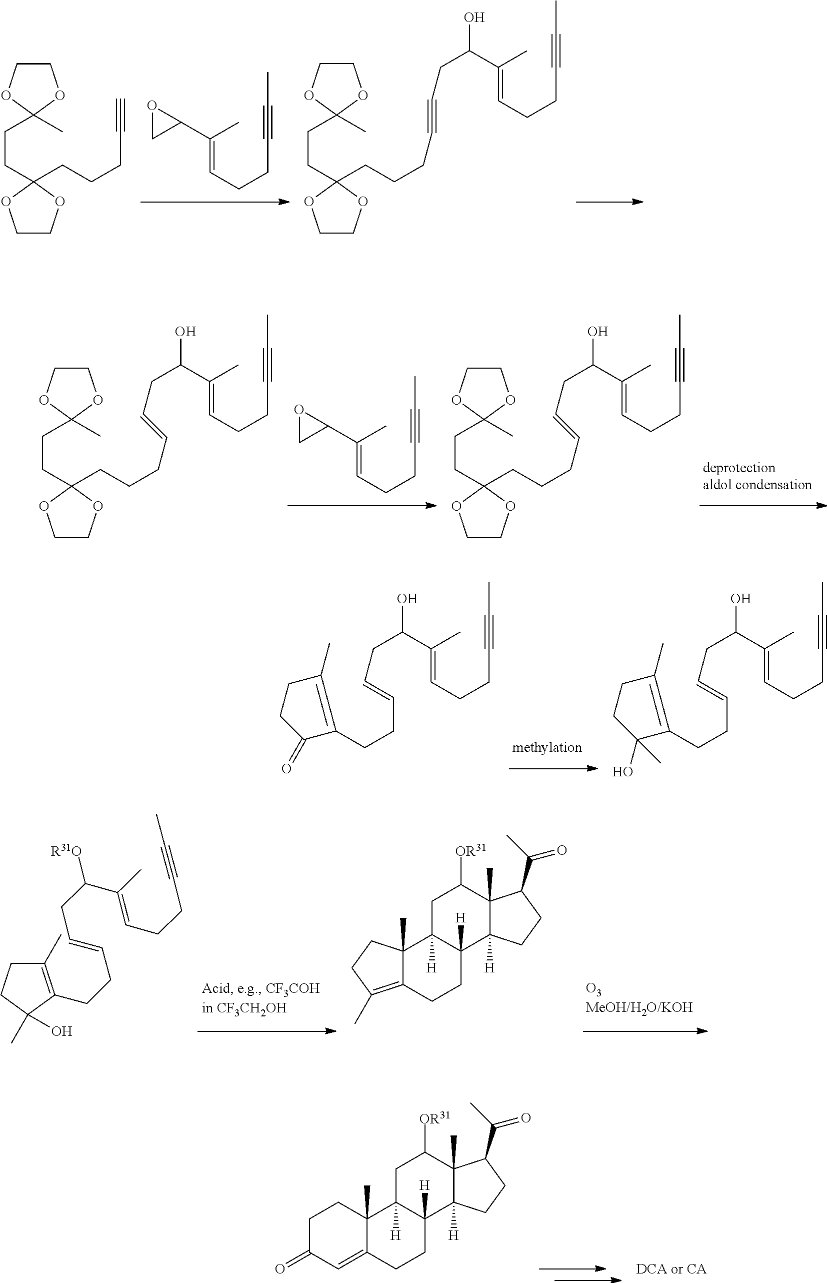

Certain bile acids of this invention can be prepared by one of several routes dependent upon the particular bile acid to be synthesized. A synthesis for cholic acid 16 from hydrocortisone 1 is described below. It is understood that cortisone is available both from modification of plant sourced steroids and by total synthesis.

Also provided is a method for preparing cholic acid 16:

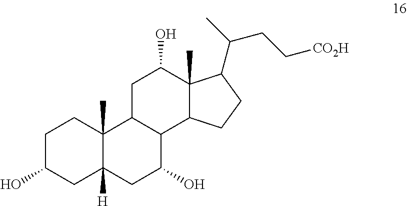 [SMILES: CC(CCC(=O)O)C1CCC2C3C(C[C@H](O)[C@]12C)[C@@]1(C)CC[C@@H](O)C[C@H]1C[C@H]3O]

said method comprising
(a) contacting hydrocortisone 1 with formaldehyde under conditions to form compound 2

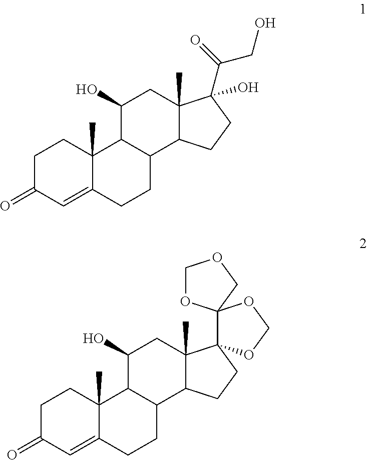

(b) contacting compound 2 with ethane-1,2-diol under conditions to form compound 3

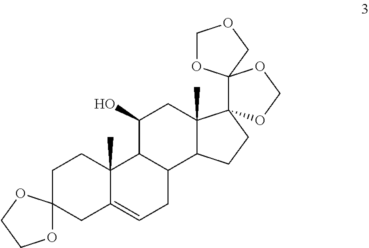

(c) contacting compound 3 with an oxidizing agent under conditions to form compound 4

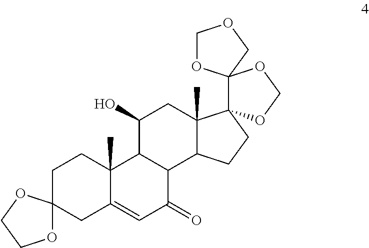

(d) contacting compound 4 with H₂ under conditions to form compound 5

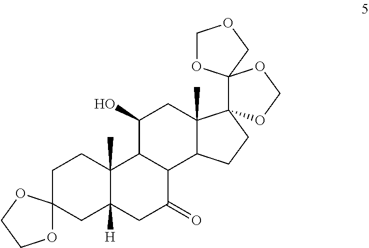

(e) contacting compound 5 with a reducing agent under conditions to form compound 6a

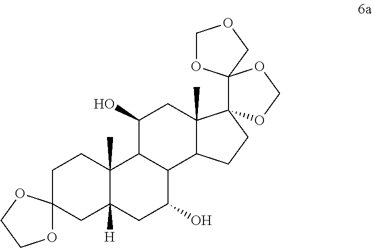

(f) converting compound 6a to compound 6 wherein P is a protecting group

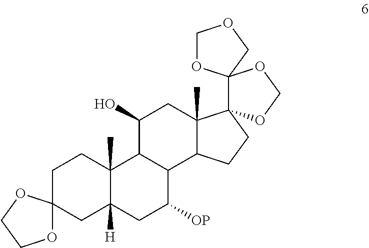

(g) contacting compound 6 under elimination conditions to form compound 7 wherein P is a protecting group

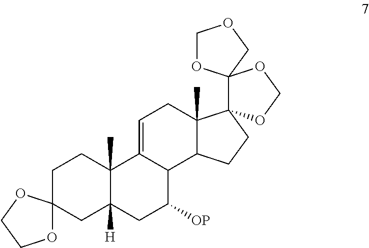

(h) contacting compound 7 with an oxidizing agent to form compound 8a wherein P is a protecting group

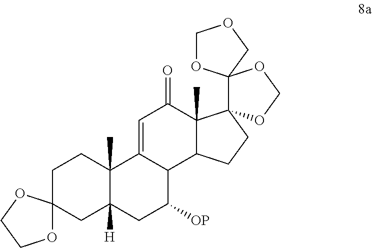

(i) contacting compound 8a with H₂ under conditions to form compound 9 wherein P is a protecting group

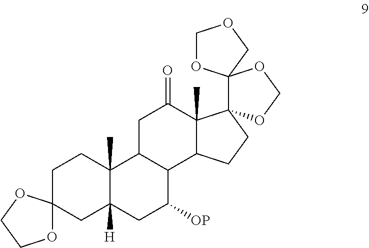

(j) contacting compound 9 with a reducing agent under conditions to form compound 10 wherein P is a protecting group

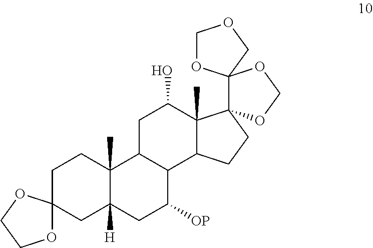

(k) contacting compound 10 with an acid to form compound 11

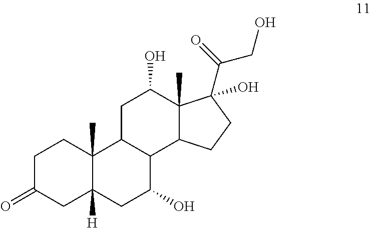 [SMILES: C[C@]12CCC(=O)C[C@H]1C[C@@H](O)C1C2C[C@H](O)[C@@]2(C)C1CC[C@]2(O)C(=O)CO]

(l) contacting compound 11 with a reducing agent under reducing conditions to form compound 12

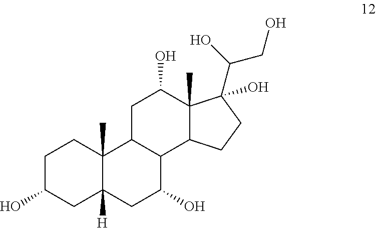 [SMILES: C[C@]12CC[C@@H](O)C[C@H]1C[C@@H](O)C1C2C[C@H](O)[C@@]2(C)C1CC[C@]2(O)C(O)CO]

(m) contacting compound 12 with a vicinal alcohol oxidizing agent to form compound 13

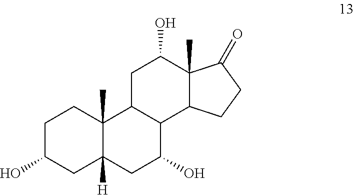 [SMILES: C[C@]12CC[C@@H](O)C[C@H]1C[C@@H](O)C1C2C[C@H](O)[C@]2(C)C(=O)CCC12]

(n) contacting compound 13 with a two carbon olefination reagent under olefin forming conditions to form compound 14

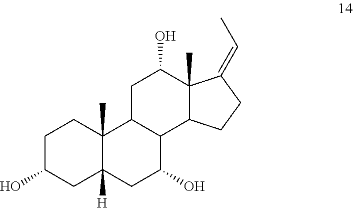 [SMILES: C/C=C1/CCC2C3C(C[C@H](O)[C@]12C)[C@@]1(C)CC[C@@H](O)C[C@H]1C[C@H]3O]

(o) contacting a compound of formula 14 with an alkyl propiolate CH═CC(O)OR¹⁰ or an alkyl acrylate CH₂═CHC(O)OR¹⁰ wherein R¹⁰ is C₁-C₆ alkyl in the presence of a Lewis acid to form a compound of formula 15 wherein the dashed line [symbol] is a single or double bond;

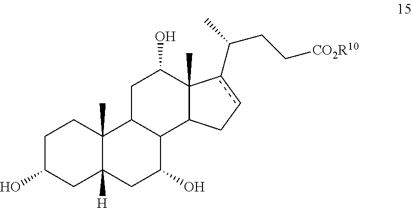

(p) contacting compound 15 wherein the dashed line [symbol] is a double bond with H₂ under hydrogenation conditions to form 16a

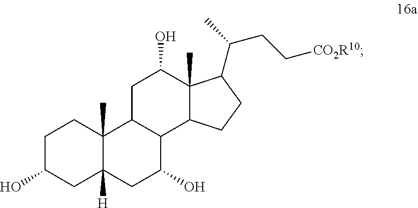

and
(q) exposing compound 16a to hydrolysis conditions to form cholic acid 16.

In one embodiment, the acid of part (a) is a mineral acid. In some embodiments, the mineral acid is HCl or H₂SO₄.

In one embodiment, the acid of part (b) is an organic acid. In some embodiments, the organic acid is a sulfonic acid such as p-toluenesulfonic acid.

In one embodiment, the oxidizing agent of parts (c) and/or (h) are selected from the group consisting of Jones reagent, tert-butyl hydroperoxide, sodium hypochlorite, hypochlorous acid, pyridinium chlorochromate, and CrO₃.

In one embodiment, the oxidation of compound 7 provides a mixture comprising one or more of compounds 8a, 8b, and 8c, wherein P is a protecting group and R³² is alkyl. Compounds of formula 8b and 8c can then be converted to compound 8a using a secondary oxidizing agent, such as NaOCl, palladium on charcoal in the presence of a base such as sodium bicarbonate, alkylhydroperoxide with cooxidants such as copper (I) iodide (CuI). In some embodiments, the secondary oxidizing agent is palladium on charcoal and a base.

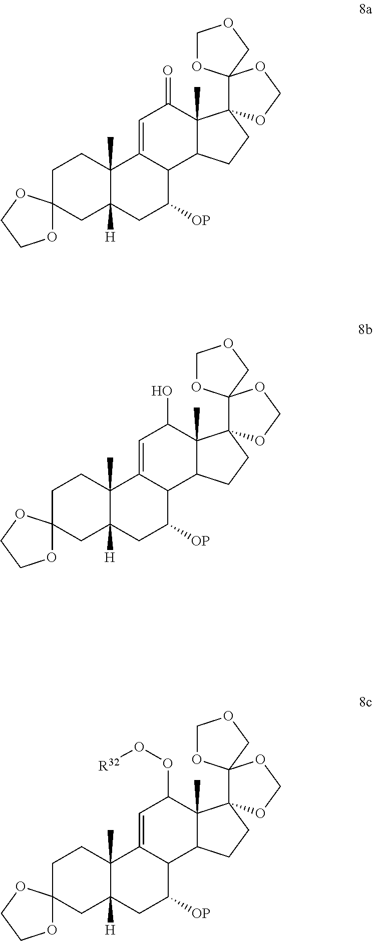

In one embodiment, the hydrogenation conditions of parts (d), (i), and/or (p) comprise a PtO₂ or Pd/C catalyst.

In one embodiment, the reducing agent of parts (e) and/or (l) is NaBH₄.

In one embodiment, the protecting group P of compounds 6a-10 is —C(O)CH₃. In some embodiments compound 5 is exposed to acylation conditions to form 6a, such as by treatment of 5 with acetic anhydride or acetylchloride and an organic base such as Et₃N, pyridine, and/or dimethylaminopyridine.

In one embodiment, the elimination conditions of part (g) comprise halogenation/elimination reaction conditions. In certain embodiments, the elimination conditions comprise converting the 11-hydroxy group of compound 6 to the corresponding 11-halo compound in the presence of an organic base such as Et₃N, pyridine, and/or dimethylaminopyridine. In some embodiments, the 11-halo compound 6 is the 11-chloro compound 6. In one embodiment, the elimination conditions of part (g) comprise POCl₃.

In one embodiment, the reducing agent of part (j) is LiAl(OtBu)₃H.

In one embodiment, the oxidizing agent of part (m) is a vicinal alcohol oxidizing agent. In some embodiments, the oxidizing agent of part (m) is a hypervalent iodide (e.g. HIO₄) or NaBiO₄.

In one embodiment, the two carbon olefination reagent of part (n) is a Wittig reagent such as Ph₃P═CH—CH₃.

In one embodiment, the Lewis acid of part (o) is EtAlCl₂.

In one embodiment, the alkyl propiolate of part (o) is methyl propriolate.

In one embodiment, the alkyl acrylate of part (o) is methyl acrylate.

Other bile acids of formula I can be prepared by the synthetic methods disclosed herein above. For example, chenodeoxycholic acid 23 can be prepared from intermediate 7 as shown in Scheme 3. An alternative route to cholic acid 16 is also shown in Scheme 3 from compound 22. In Scheme 3, synthetic steps d, f, k, l, m, n, o, p, q, and i are as described above.

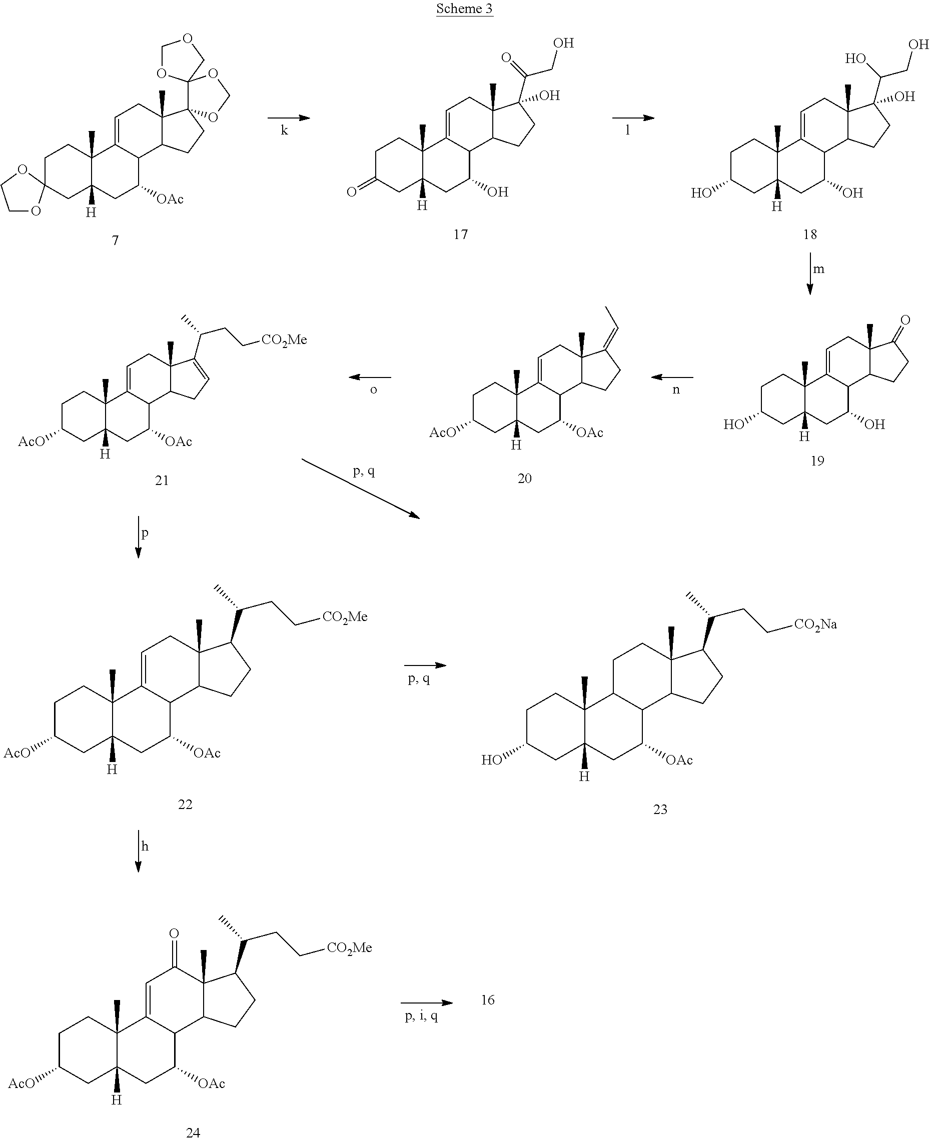

Various other compounds of formula I can be prepared according to Scheme 4. For example, lithocholic acid 30 can be prepared from intermediate 23a as shown below in Scheme 4. Specifically, compound 26 can be prepared from compound 23a under acidic reaction conditions. In one embodiment, the acidic reaction conditions comprise HCl. Monoprotection of the less hindered 3-hydroxy group of compound 26 using a suitable protecting group, P, yields compound 27. In one embodiment, protecting group P is tert-butylsilyl ether. Reacting compound 27 under deoxygenation conditions provides compound 28. In one embodiment, the deoxygenation conditions comprise radical-initiated deoxygenation conditions (e.g. Barton-McCombie deoxygenation) via the corresponding 7-thiocarbonyl derivative of compound 27. Deprotection of the 3-hydroxy group of compound 28 provides compound 29. In one embodiment, the deprotection of the 3-hydroxy group of compound 28 comprises a fluoride source. Finally, hydrolysis of the methyl ester of compound 29 provides Lithocholic acid 30. In one embodiment, the hydrolysis comprises an aqueous base (e.g. LiOH).

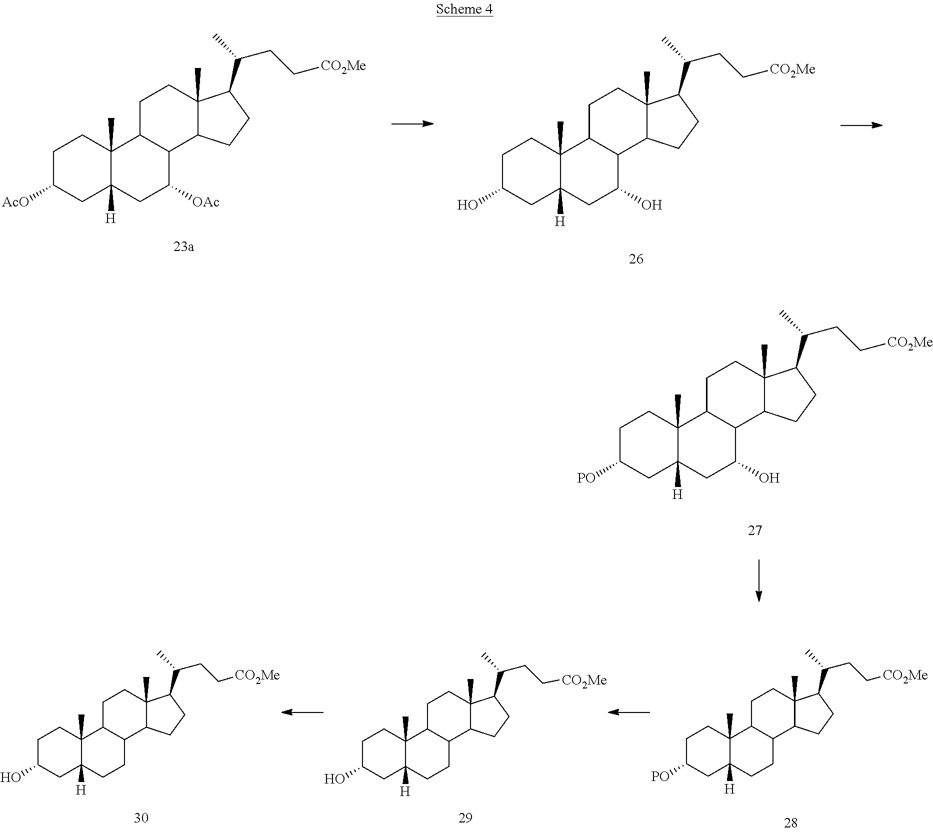

It is contemplated that the compounds disclosed herein can be used for the preparation of other bile acid derivatives, such as deoxycholic acid (DCA). For example, DCA (70) can be synthesized by:

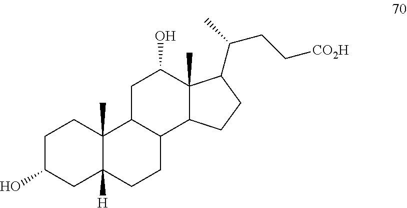 [SMILES: C[C@H](CCC(=O)O)C1CCC2C3CC[C@@H]4C[C@H](O)CC[C@]4(C)C3C[C@H](O)[C@@]21C]

(a) contacting compound 3 with H₂ under conditions to form compound 60

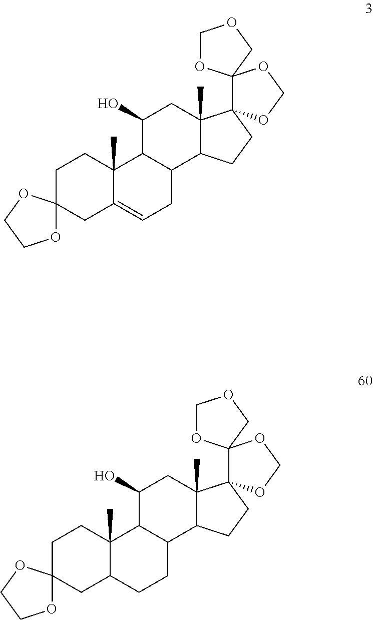

(b) contacting compound 60 under elimination conditions to form compound 61

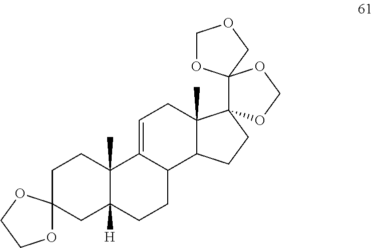

(c) contacting compound 61 with an oxidizing agent to form compound 62

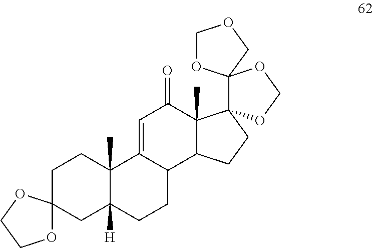

(d) contacting compound 62 with H₂ under conditions to form compound 63

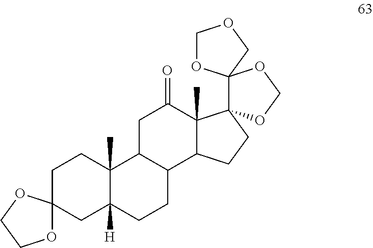

(e) contacting compound 63 with a reducing agent under conditions to form compound 64

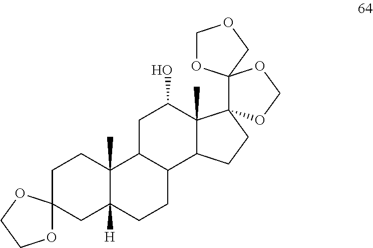

(f) contacting compound 64 with an acid to form compound 65

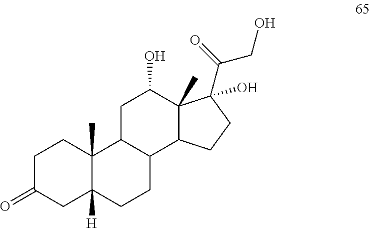 [SMILES: C[C@]12CCC(=O)C[C@H]1CCC1C2C[C@H](O)[C@@]2(C)C1CC[C@]2(O)C(=O)CO]

(g) contacting compound 65 with a reducing agent under reducing conditions to form compound 66

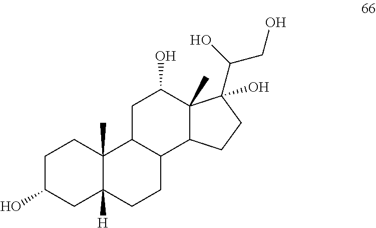 [SMILES: C[C@]12CC[C@@H](O)C[C@H]1CCC1C2C[C@H](O)[C@@]2(C)C1CC[C@]2(O)C(O)CO]

(h) contacting compound 66 with a vicinal alcohol oxidizing agent to form compound 67

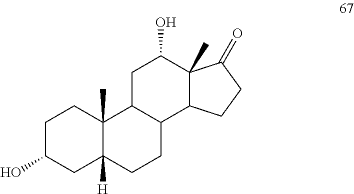 [SMILES: C[C@]12CC[C@@H](O)C[C@H]1CCC1C2C[C@H](O)[C@]2(C)C(=O)CCC12]

(i) contacting compound 67 with a two carbon olefination reagent under olefin forming conditions to form compound 68

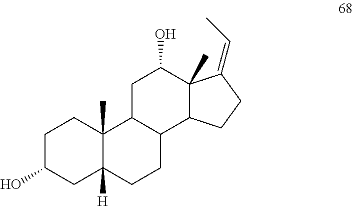 [SMILES: C/C=C1/CCC2C3CC[C@@H]4C[C@H](O)CC[C@]4(C)C3C[C@H](O)[C@]12C]

(j) contacting a compound of formula 68 with an alkyl propiolate CH≡CC(O)OR¹⁰ or an alkyl acrylate CH₂═CHC(O)OR¹⁰ wherein R¹⁰ is C₁-C₆ alkyl in the presence of a Lewis acid to form a compound of formula 69 wherein the dashed line [symbol] is a single or double bond;

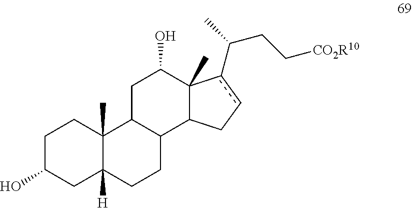

(k) contacting compound 69 wherein the dashed line [symbol] is a double bond with H₂ under hydrogenation conditions to form 70a

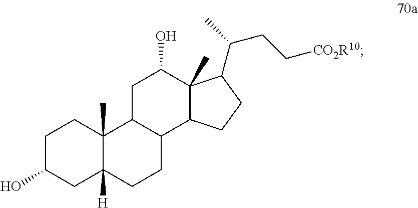

and
(l) exposing compound 70a to hydrolysis conditions to form deoxycholic acid 70.

Various novel intermediates are disclosed in the synthetic methods described herein. Accordingly, one embodiment of the present invention is directed to such intermediates (i.e., compounds 1, 3, 4, 5, 6, 6a, 7, 8a, 9, 10, 11, 12, 13, 14, 15, 16a, 17, 18, 19, 20, 21, 23, 24, 26, 27, 28, 29, 60, 61, 62, 63, 64, 65, 66, 67, and 68).

Further compounds of formula VII, represented by formula VIIB, can be prepared from the compounds disclosed above according to Scheme 5 using standard coupling reaction conditions well known in the art. In Scheme 5, R³, R⁷, R⁹, w¹, w², W, and t are as defined herein.

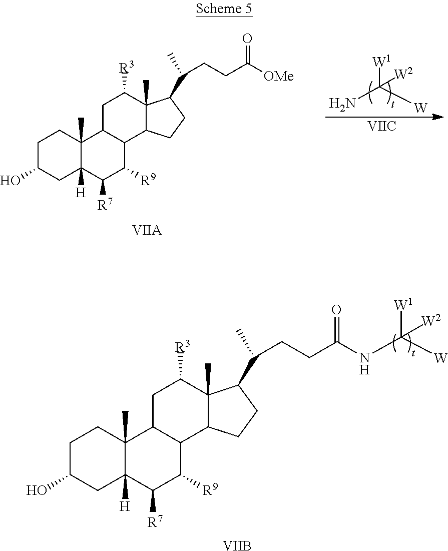

In certain embodiments, the compound of formula VIIA in Scheme 5 is selected from the group consisting of cholic acid, chenodeoxycholic acid and lithocholic acid. In some embodiments, the cholic acid, chenodeoxycholic acid and lithocholic acid are prepared using the synthetic methods disclosed herein. Specific examples of the transformations shown in Scheme 5 are shown below in Scheme 6, wherein P is a protecting group such as alkyl or substituted alkyl, preferably tertiary butyl or benzyl. For example, cholic acid 16 can be converted to the glycine conjugate 31 using carboxy-protected glycine (commercially available from Aldrich®, USA) under standard coupling reaction conditions. Similarly, the taurine conjugate 32 of cholic acid 16 can be synthesized using the protected taurine derivative (commercially available from Aldrich®, USA) under standard coupling reaction conditions.

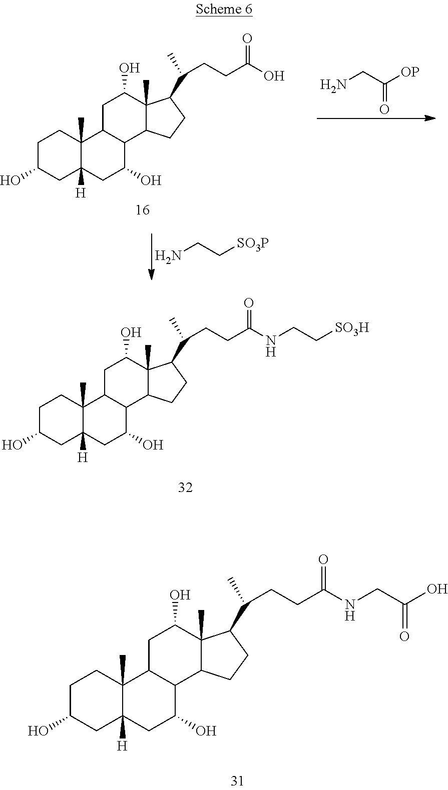

Also disclosed herein are dendritic compounds of formula VIII. Such compounds are provided from compound VIIA according to Scheme 7 under typical coupling reaction conditions. In certain embodiments, the compound of formula VIIA in Scheme 7 is selected from the group consisting of cholic acid, chenodeoxycholic acid and lithocholic acid. In some embodiments, the cholic acid, chenodeoxycholic acid and lithocholic acid are prepared using the synthetic methods disclosed herein. Specifically, tripodalcholamine derivative 33 can be prepared from the reaction of at least a three-fold excess of cholic acid 16 with N,N-bis(aminomethyl)methanediamine. Such dendritic compounds are useful in the preparation of hydrogel and hydrogel-like materials. In Scheme 7, R³, R⁷, and R⁸ are as disclosed above.

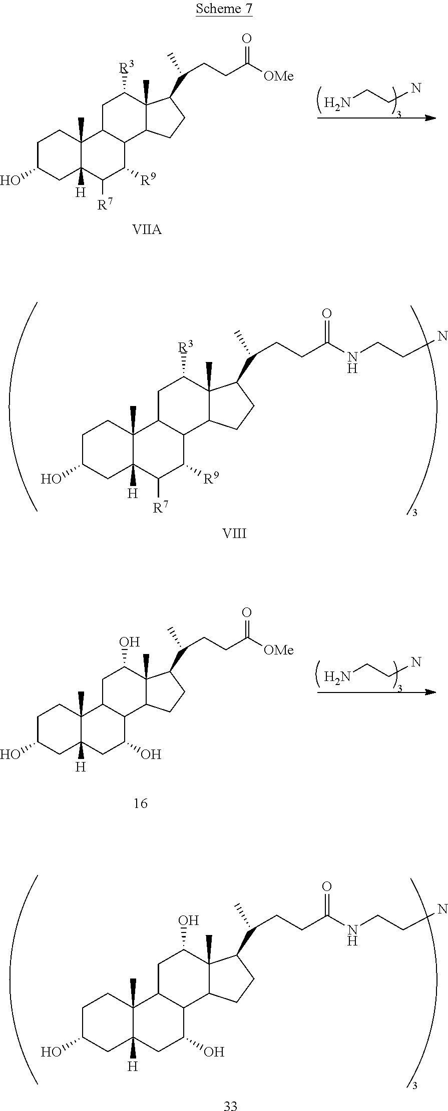

Further compounds of formula VII can be prepared using the methods disclosed herein and shown in Scheme 8, where P is a protecting group and R⁷¹ is alkyl.

In scheme 8, compound 34 can be prepared via selective oxidation of the 7-hydroxy group of synthetic cholic acid 16 as disclosed herein. Esterification of the carboxyl group of compound 34 yields compound 35. Alternatively, compound 35 can be prepared via selective oxidation of the 7-hydroxy group of intermediate 16a. Contacting compound 35 with TMSCl and triethylamine yields enol ether 36, which reacts with an aldehyde of the formula R²¹CHO in the presence of a Lewis acid (e.g. BF₃OEt₂) provides compound 37. Reduction of the 7-ene of compound 37 using hydrogen gas with a suitable catalyst (e.g. PtO₂) followed by hydrolysis of the methyl ester yields compound 38. Reduction of the 7-oxo of compound 38 using a suitable hydride reagent (e.g. NaBH₄) yields compound 39. Conversion of the carboxyl group of compound 39 to the corresponding methyl ester and protection of the hydroxy groups with a suitable protecting group P gives compound 40. Non-stereoselective methylation at C-23 (using a base and methyl iodide) yields compound 41 as a mixture of epimers. Hydrolysis of the methyl ester followed by separation of the diastereomers using conventional chiral separation methods provides S-42 and R-42. A single stereoisomer may also be provided at C-23 via deprotonation/reprotonation using a chiral proton source where such methods are known in the art.

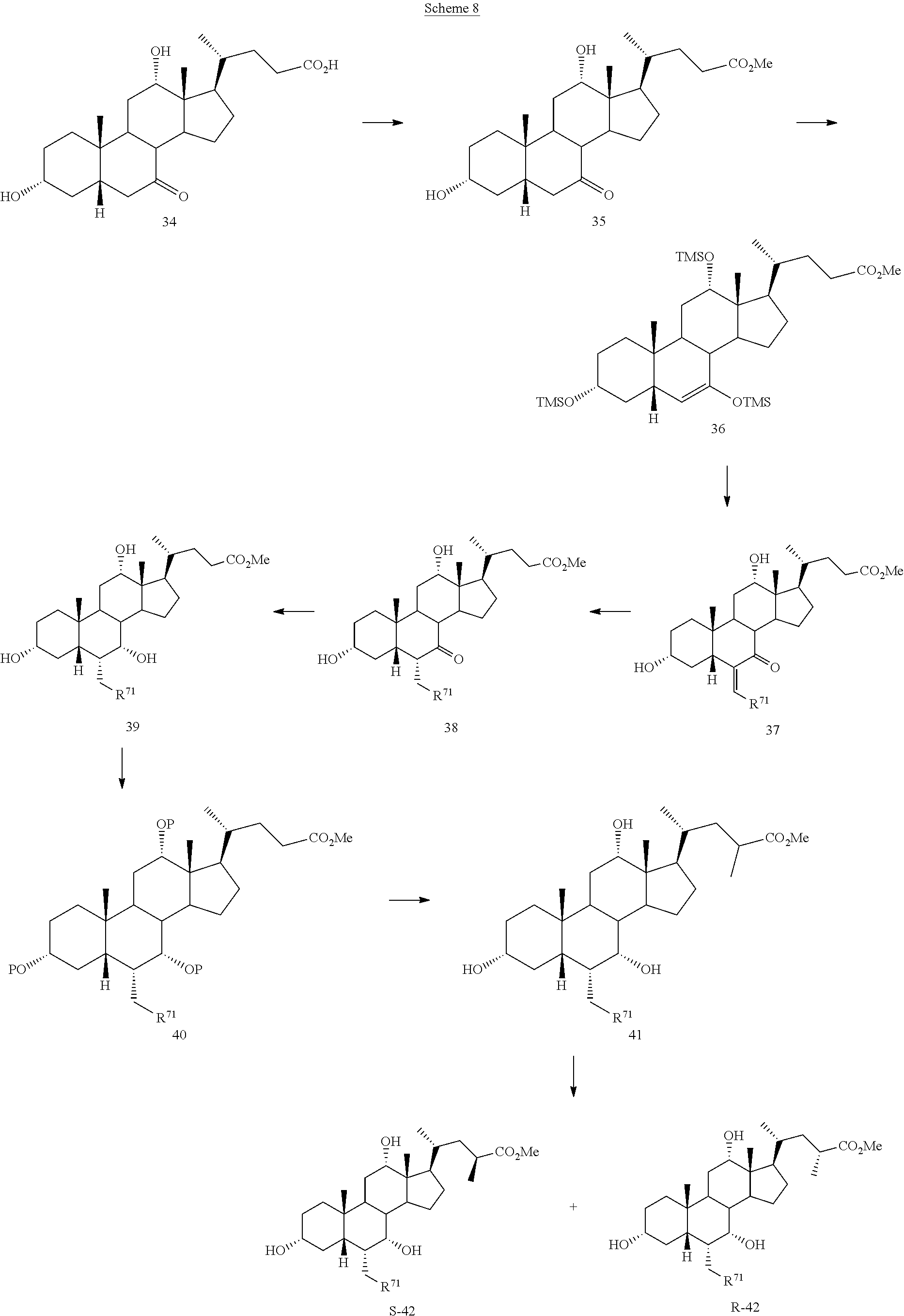

The synthetic methods exemplified in Scheme 8 can be extended to various other bile acids which can be prepared using the methods disclosed herein. Examples of such compounds are shown below in Scheme 9. Such compounds can be prepared using methods well known in the art from compounds such as chenodeoxycholic acid and cholic acid. It is contemplated that these compounds will be useful as FXR active compounds.

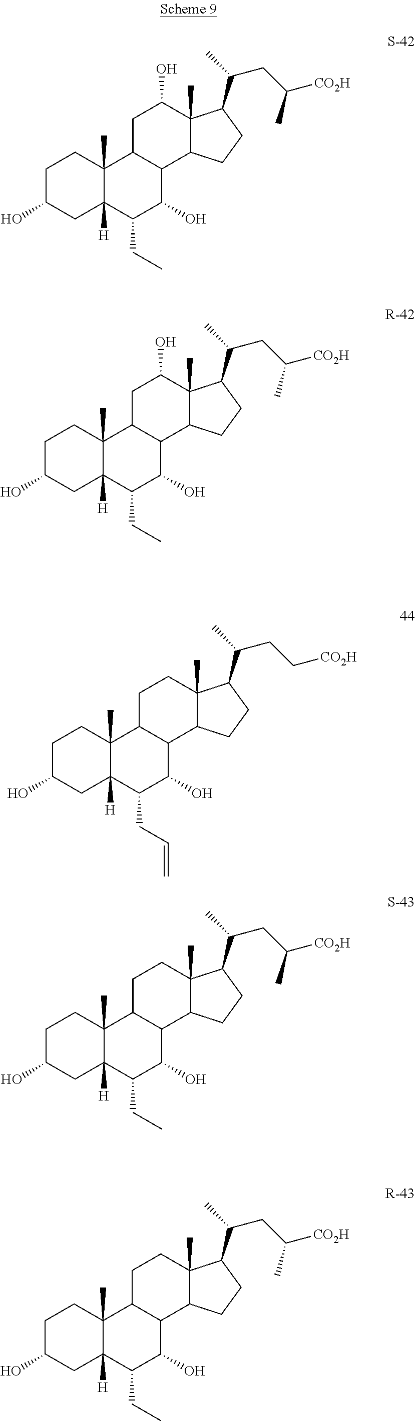 [SMILES: CC[C@H]1[C@@H](O)C2C3CC[C@H]([C@H](C)C[C@H](C)C(=O)O)[C@@]3(C)[C@@H](O)CC2[C@@]2(C)CC[C@@H](O)C[C@@H]12 ; CC[C@H]1[C@@H](O)C2C3CC[C@H]([C@H](C)C[C@@H](C)C(=O)O)[C@@]3(C)[C@@H](O)CC2[C@@]2(C)CC[C@@H](O)C[C@@H]12 ; C=CC[C@H]1[C@@H](O)C2C3CC[C@H]([C@H](C)CCC(=O)O)[C@@]3(C)CCC2[C@@]2(C)CC[C@@H](O)C[C@@H]12 ; CC[C@H]1[C@@H](O)C2C3CC[C@H]([C@H](C)C[C@H](C)C(=O)O)[C@@]3(C)CCC2[C@@]2(C)CC[C@@H](O)C[C@@H]12 ; CC[C@H]1[C@@H](O)C2C3CC[C@H]([C@H](C)C[C@@H](C)C(=O)O)[C@@]3(C)CCC2[C@@]2(C)CC[C@@H](O)C[C@@H]12]

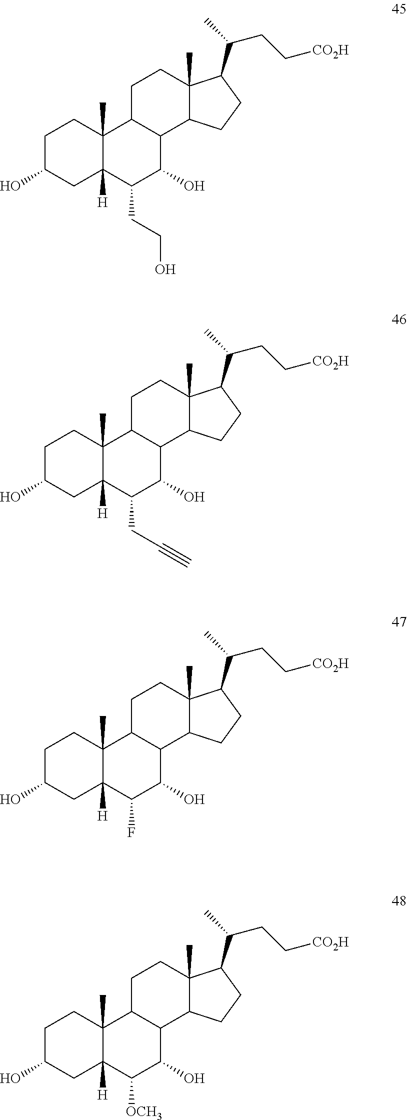 [SMILES: C[C@H](CCC(=O)O)[C@H]1CCC2C3C(CC[C@@]21C)[C@@]1(C)CC[C@@H](O)C[C@H]1[C@@H](CCO)[C@H]3O ; C#CC[C@H]1[C@@H](O)C2C3CC[C@H]([C@H](C)CCC(=O)O)[C@@]3(C)CCC2[C@@]2(C)CC[C@@H](O)C[C@@H]12 ; C[C@H](CCC(=O)O)[C@H]1CCC2C3C(CC[C@@]21C)[C@@]1(C)CC[C@@H](O)C[C@H]1[C@@H](F)[C@H]3O ; CO[C@H]1[C@@H](O)C2C3CC[C@H]([C@H](C)CCC(=O)O)[C@@]3(C)CCC2[C@@]2(C)CC[C@@H](O)C[C@@H]12]

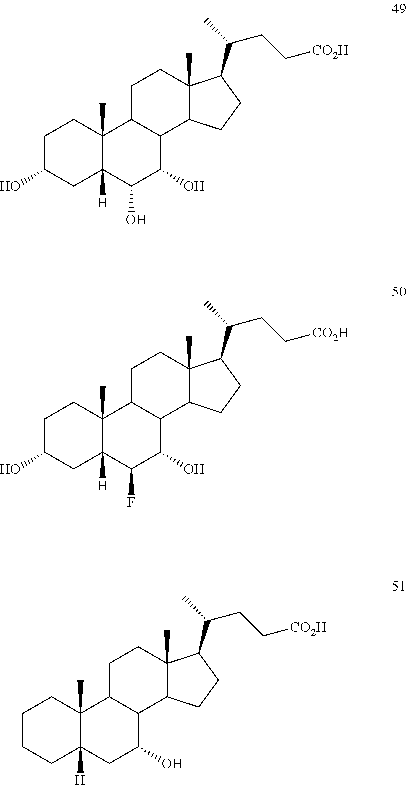 [SMILES: C[C@H](CCC(=O)O)[C@H]1CCC2C3C(CC[C@@]21C)[C@@]1(C)CC[C@@H](O)C[C@H]1[C@@H](O)[C@H]3O ; C[C@H](CCC(=O)O)[C@H]1CCC2C3C(CC[C@@]21C)[C@@]1(C)CC[C@@H](O)C[C@H]1[C@H](F)[C@H]3O ; C[C@H](CCC(=O)O)[C@H]1CCC2C3C(CC[C@@]21C)[C@@]1(C)CCCC[C@H]1C[C@H]3O]

The synthetic methods exemplified herein can further be extended to prepare the bile acid derivatives shown in Scheme 10 and Table 1. Such compounds can be prepared using methods well known in the art from compounds such as chenodeoxycholic acid and cholic acid.

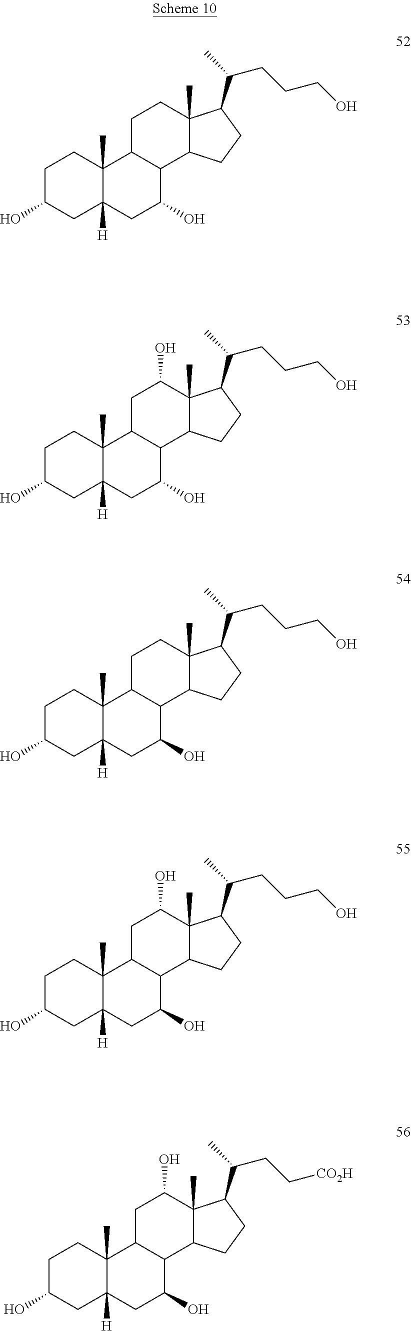 [SMILES: C[C@H](CCCO)[C@H]1CCC2C3C(CC[C@@]21C)[C@@]1(C)CC[C@@H](O)C[C@H]1C[C@H]3O ; C[C@H](CCCO)[C@H]1CCC2C3C(C[C@H](O)[C@@]21C)[C@@]1(C)CC[C@@H](O)C[C@H]1C[C@H]3O ; C[C@H](CCCO)[C@H]1CCC2C3C(CC[C@@]21C)[C@@]1(C)CC[C@@H](O)C[C@H]1C[C@@H]3O ; C[C@H](CCCO)[C@H]1CCC2C3C(C[C@H](O)[C@@]21C)[C@@]1(C)CC[C@@H](O)C[C@H]1C[C@@H]3O ; C[C@H](CCC(=O)O)[C@H]1CCC2C3C(C[C@H](O)[C@@]21C)[C@@]1(C)CC[C@@H](O)C[C@H]1C[C@@H]3O]

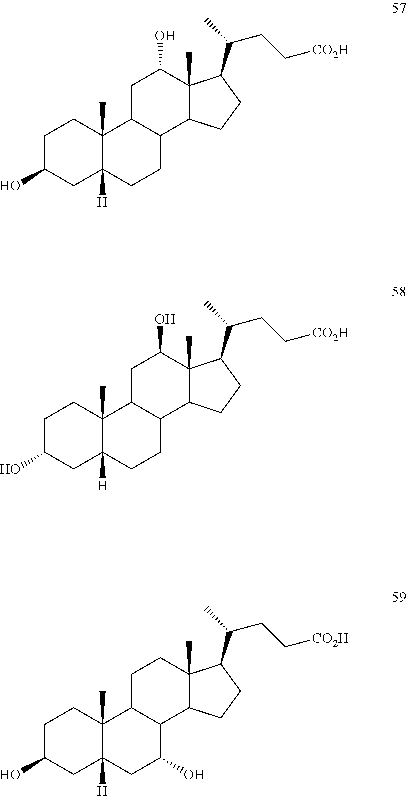 [SMILES: C[C@H](CCC(=O)O)[C@H]1CCC2C3CC[C@@H]4C[C@@H](O)CC[C@]4(C)C3C[C@H](O)[C@@]21C ; C[C@H](CCC(=O)O)[C@H]1CCC2C3CC[C@@H]4C[C@H](O)CC[C@]4(C)C3C[C@@H](O)[C@@]21C ; C[C@H](CCC(=O)O)[C@H]1CCC2C3C(CC[C@@]21C)[C@@]1(C)CC[C@H](O)C[C@H]1C[C@H]3O]

TABLE 1

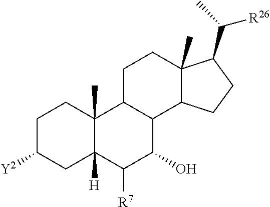

| No. | R¹ | R⁷ | R²⁶ |
|---|---|---|---|
| i | OH | H | 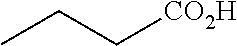 |
| ii | OH | H | 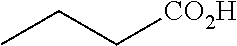 |
| iii | OH | α-CH₃ | 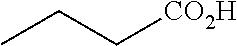 |
| iv | OH | α-CH₂CH₃ | 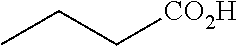 |
| v | OH | 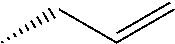 | 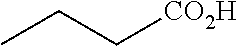 |
| vi | OH | 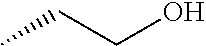 | 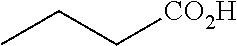 |
| vii | OH | 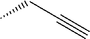 | 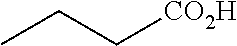 |
| viii | OH | α-OH | 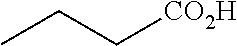 |
| ix | OH | α-OCH₃ | 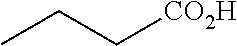 |
| x | OH | α-F | 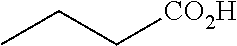 |
| xi | OH | β-F | 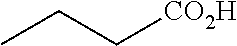 |
| xii | OH | H | 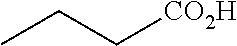 |
| xiii | OH | H | 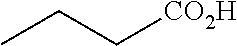 |
| xiv | OH | H | 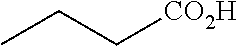 |
| xv | OH | H | 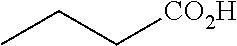 |
| xvi | H | H | 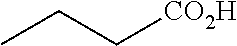 |
| xvii | OH | H | 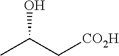 |
| xviii | OH | H | 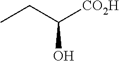 |
| xix | OH | H | 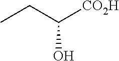 |
| xx | OH | H | 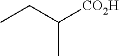 |
| xxi | OH | H | 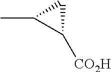 |
| xxii | OH | H | 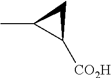 |
| xxiii | OH | H | 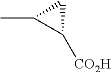 |
| xxiv | OH | H | 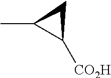 |
| xxv | OH | H | —CO₂H |
| xxvi | OH | H | 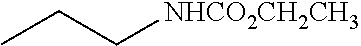 |
| xxvii | OH | H | 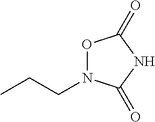 |
| xxviii | OH | H | 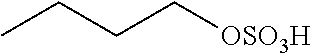 |
| xxix | OH | H | 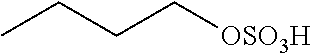 |
| xxx | OH | H | 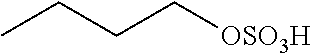 |

It is understood that one of skill in the art could use the synthetic cholic acid disclosed herein for various pharmaceutical uses including those described herein below, as well as for the preparation of various known bile acids and/or novel derivatives thereof. For example, one of skill in the art would readily envision selective protection/deprotection of the various hydroxy groups of cholic acid (see, Greene, supra). Such chemistry paired with one or more synthetic modifications, such as dehydration, oxidation, substitution, etc., would provide various known bile acids and/or novel derivatives thereof such as those disclosed in Table 1, above.

Specifically, the C-12 and C-3 hydroxy groups of cholic acid can be selectively protected and the C-7 hydroxy group utilized as a synthetic handle for the preparation of derivatives at C-6 (i.e., R⁷ of formula VII:

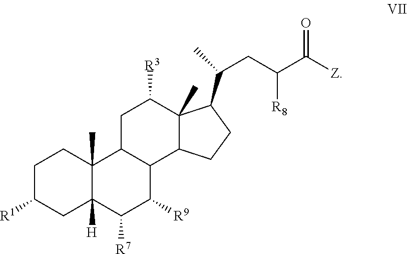

Synthesizing cholic acid from deoxycholic acid or from 3-oxo-4,5-ene steroids according to this invention is provided below.

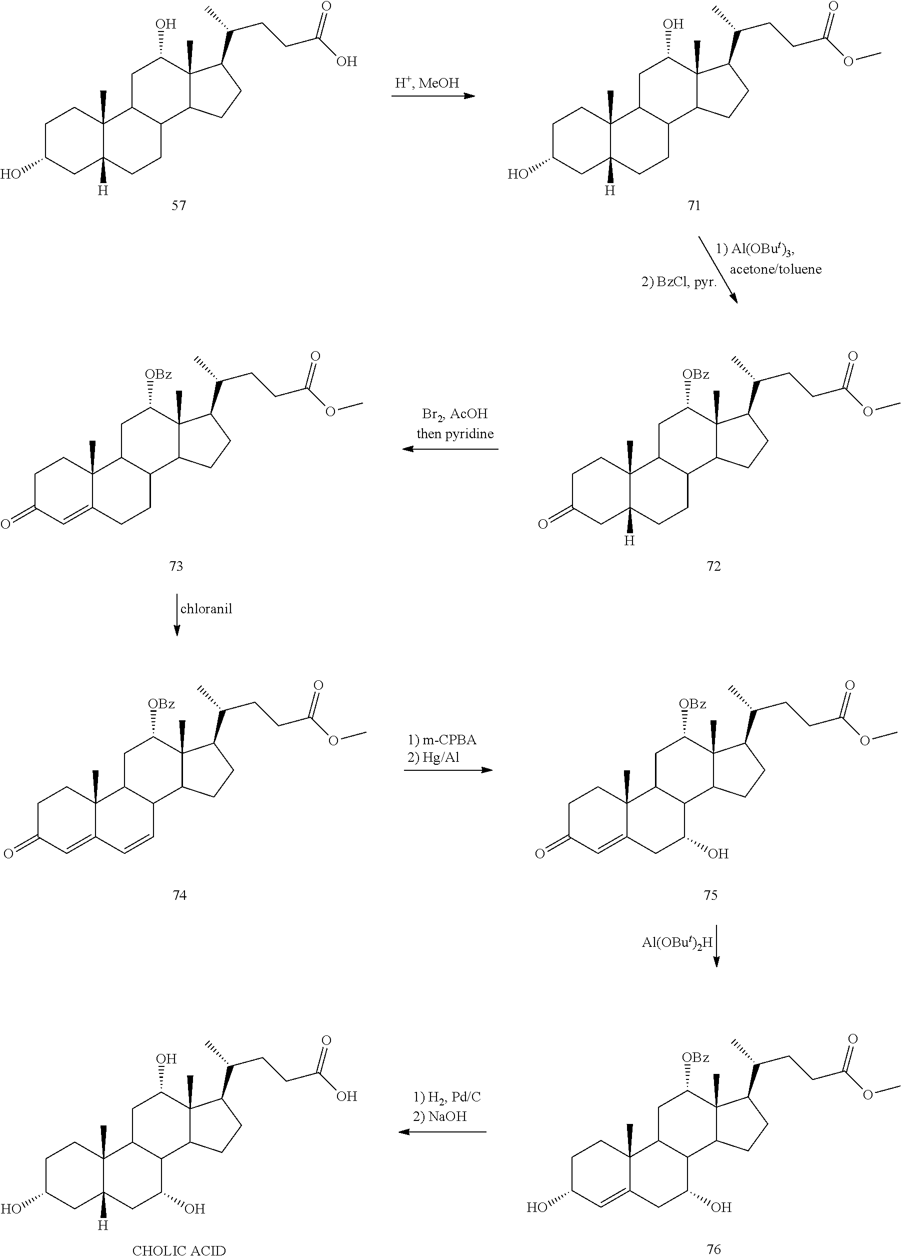

For illustration, and not for limitation, the 3-oxo-4,5-ene steroid utilized here is a compound of formula 4, 5, or 6. However, other such steroids, for example, those without the C-17 bile acid side chain are converted to cholic acid in a similar manner and the C-17 sidechain incorporated following other methods described here or known to the skilled artisan.

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

The starting materials and reagents for the reactions described herein are generally known compounds or can be prepared by known procedures or obvious modifications thereof. For example, many of the starting materials and reagents are available from commercial suppliers such as Aldrich Chemical Co. (Milwaukee, Wis., USA), Bachem (Torrance, Calif., USA), Emka-Chem or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds prepared according to this invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

### Compounds

In another aspect, this invention provides synthetic bile acid of formula VII:

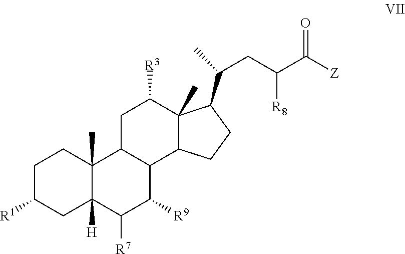

wherein:
R⁷ is hydrogen, halo, alkyl, alkenyl, alkynyl, or alkoxy;
R⁸ is hydrogen, halo, alkyl, alkenyl, alkynyl, alkoxy, or haloalkyl;
R¹, R³, and R⁹ are each independently hydrogen, hydroxy, or alkoxy;
Z is hydroxy, alkoxy, —NH₂, or

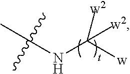

where t is 1 or 2, w¹ and w² are each independently H or (C₁₋₄)alkyl optionally substituted with hydroxy, alkoxy, thio, thioalkyl, amino, substituted amino, aryl, and substituted aryl, and W is —COOH or —SO₃H; or a salt thereof;
provided that when R⁷ and R⁸ are hydrogen and R⁹ and Z are hydroxy, then R³ is not hydroxy.

This invention also provides novel intermediates useful for synthesizing bile acids. In certain preferred embodiments, the following compounds are provided:

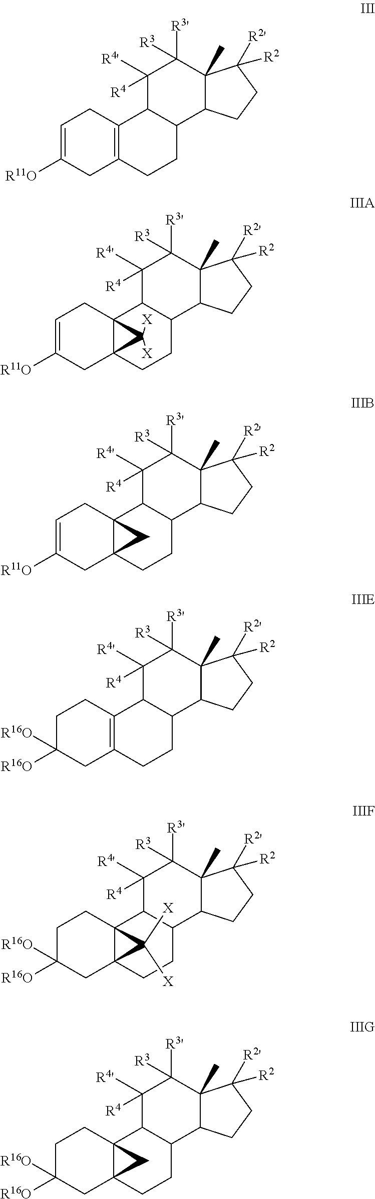

wherein the substituents are defined below.

In one embodiment, R¹¹ and R¹⁶ are substituted or unsubstituted alkyl, alkenyl, or alkynyl, or two R¹⁶ groups together with the oxygen atoms they are attached to form a cyclic ketal. Preferably, R¹⁶ is unsubstituted alkyl, or two R¹⁶ groups together with the oxygen atoms they are attached to form a 5 or 6 membered cyclic ketal. In another embodiment, R¹¹ or R¹⁶ is methyl, ethyl, allyl, benzyl, or the like.

In another embodiment, R^{2′} is H and R² is hydroxy, substituted or unsubstituted alkyl, alkenyl, or alkynyl, or is —OR²², —COR²², or —OCOR²²; or R² and R^{2′} together with the carbon atom they are bonded to form a cyclic ketal, or CR²R^{2′} is oxo or C═CR²³R²⁴. In another embodiment, R²² is alkyl. In another embodiment, R²² is a hydroxy substituted alkyl. In another embodiment, R²² is methyl. Preferably, R^{2′} is H and R² is hydroxy, or —OR²²; or R² and R^{2′} together with the carbon atom they are bonded to form a cyclic ketal, or CR²R^{2′} is oxo.

In another embodiment, R^{3′} is H and R³ is hydroxy, —OR³¹, or —OCOR³¹; or R³ and R^{3′} together with the carbon atom they are bonded to form a cyclic ketal, or CR³R^{3′} is oxo. In another embodiment, R^{3′} is H, and R³ is an alpha or beta hydroxy, OR³¹, or is —OCOR³¹. In another embodiment, R³¹ is methyl, ethyl, allyl, benzyl, or the like.

In another embodiment, R⁴ and R^{4′} are H.

### Therapeutic Methods

It is contemplated that the bile acids and derivatives thereof disclosed herein are active at the FXR receptor (see U.S. Pat. No. 6,005,086; U.S. Pat. No. 6,465,258; WO/2000/037077, each of which are incorporated herein in their entirety). It has also been shown that compounds which are active at the FXR receptor are active in modulating cholesterol and/or fat metabolism by regulating FXR activity (See U.S. Pat. No. 7,705,028).

It is further contemplated that one or more of the compounds disclosed herein can be used for localized fat removal as per U.S. Pat. No. 7,622,130; U.S. 2005/0267080; U.S. 2006/127468; and U.S. 2006/0154906. Accordingly, in one embodiment, the present invention is directed to the decrease or removal of localized fat accumulation in patients by providing a non-surgical method for removing fat deposits by administration of fat-solubilizing concentrations of the bile acids disclosed herein in pharmaceutically acceptable formulations.

For the purposes of the present invention, a non-surgical method of fat removal does not include liposuction, lipoplasty or suction lipectomy.

In one embodiment of the present invention, a medical composition for the non-surgical removal of localized fat deposits in a patient is provided which comprises at least one pharmacologically active bile acid compound as disclosed herein, optionally at least one pharmaceutically acceptable excipient and optionally at least one additional active ingredient wherein the medical composition does not include phosphotidylcholine. The bile salt can be at least one of deoxycholic, cholic, chenodeoxycholic, 7-alpha-dehydroxylate, chenodeoxycholic, lithocholic, ursodeoxycholic, dihydroxy- and trihydroxy-bile salts. The bile salts can be in the taurine or glycine conjugate forms.

In yet another embodiment of the present invention the medical composition contains one or more additional active ingredients. One or more additional active ingredients can include anti-inflammatory agents such as a steroidal anti-inflammatory agent or a non-steroidal anti-inflammatory agent; analgesics and dispersion agents such as hyaluronidase or collagenase.

In some embodiments, the medical composition contains one or more pharmaceutically acceptable excipients.

In some embodiments, the patient is a human.

In one embodiment of the present invention, a method is provided for the non-surgical removal of localized fat deposits in a patient having localized fat accumulation comprising administering a fat solubilizing amount of a pharmacologically active composition comprising a bile acid compound as disclosed herein, wherein the non-surgical method does not include liposuction.

In one embodiment of the present invention, the pharmacologically active bile acid composition comprises at least one pharmacologically active bile acid compound as disclosed herein, optionally at least one pharmaceutically acceptable excipient and optionally at least one additional active ingredient, and wherein the pharmacologically active bile acid composition does not contain phosphatidylcholine.

In some embodiments of the present invention, the pharmacologically active composition comprising a bile acid compound as disclosed herein is administered by subcutaneous injection directly into fat tissue.

In one embodiment of the present invention, the localized fat accumulation is lower eyelid fat herniation, lipomas, lipodystrophy, buffalo hump lipodystrophy or fat deposits associated with cellulite.

In another embodiment of the present invention, a medical composition is provided for removing localized accumulation of fat in a patient with lower eyelid fat herniation comprising a fat solubilizing amount of a bile acid compound as disclosed herein, and the medical composition does not contain phosphatidylcholine.

In an embodiment of the present invention a non-liposuction method for the non-surgical removal of localized fat deposits in a patient is provided comprising the non-surgical administration of a pharmacologically active composition consisting essentially of at least one bile acid compound as disclosed herein, optionally at least one pharmaceutically acceptable excipient and optionally at least one additional active ingredient, and the medical composition does not include phosphatidylcholine.

Compositions produced according to the present invention can include other active ingredients including, without limitation, and in any compatible combination, anti-inflammatory agents, analgesics, dispersion agents, penetration enhancers and pharmaceutically acceptable excipients.

Anti-inflammatory agents suitable for use with the compositions of the present invention can include both steroidal anti-inflammatory agents and non-steroidal anti-inflammatory agents. Suitable steroidal anti-inflammatory agent can include, although are not limited to, corticosteroids such as hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, and mixtures thereof can be used.

A second class of anti-inflammatory agents which is useful in the compositions of the present invention includes the nonsteroidal anti-inflammatory agents. The variety of compounds encompassed by this group are well-known to those skilled in the art.

Suitable non-steroidal anti-inflammatory agents useful in the compositions of the present invention include, but are not limited to: the oxicams, such as piroxicam, isoxicam, tonexicam, sudoxicam, and CP-14,304; the salicylates, such as salicylic acid, aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; the fenamates, such as mefenamic, meclofenamic, flufenamic, niflumic, and tolfenamic acids; the propionic acid derivates, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and the pyrazoles, such as phenybutazone, oxyphenbutazone, feprazone, azapropazone, and trimethazone. Mixtures of these non-steroidal anti-inflammatory agents can also be employed, as well as the pharmaceutically-acceptable salts and esters of these agents.

Analgesics suitable for use with the pharmacologically active bile acid composition of the present invention to reduce discomfort due to inflammation after subcutaneous injection of the formulation of the present invention include, but are not limited to, injectable local amine and ester anesthetics. Non-limiting examples of analgesics include lidocaine, mepivacaine, bupivacaine, procaine, chloroprocaine, etidocaine, prilocalne and tetracaine. Mixtures of these analgesics can also be employed, as well as the pharmaceutically acceptable salts and esters or these agents.

Pharmacologically acceptable aqueous vehicles for the compositions of the present invention can include, for example, any liquid solution that is capable of dissolving a compound of the invention and is not toxic to the particular individual receiving the formulation. Examples of pharmaceutically acceptable aqueous vehicles include, without limitation, saline, water and acetic acid. Typically, pharmaceutically acceptable aqueous vehicles are sterile.

Pharmacologically active bile acid compositions useful in embodiments of the present invention are formulated for the non-surgical removal of localized fat deposits. As used herein, “non-surgical” refers to medical procedures that do not require an incision. Injections are examples of non-surgical procedures. Liposuction is a surgical procedure.

In one embodiment of the present invention, the pharmacologically active bile acid composition is administered by injection, for example, by bolus injection. In order to be effective, the pharmacologically active bile acid composition must have direct contact with the fat tissue regardless of how it is infused. The pharmacologically active bile acid formulations can be injected subcutaneously or infused directly into the fat. Formulations for injection can be presented in unit dosage form, for example, in ampoules or in multi-dose containers, with an added preservative. The compositions can take such forms as suspensions, solutions, or emulsions in oily or aqueous vehicles, and can contain formulatory agents such as suspending, stabilizing and/or dispersing agents.

A “pharmaceutically acceptable excipient” means a compound that is useful in preparing a pharmaceutical composition that is generally safe, non-toxic and neither biologically nor otherwise undesirable, and includes excipients that are acceptable for veterinary use or human pharmaceutical use. A pharmaceutically acceptable excipient as used in the specification and claims includes both one and more than one such excipient. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, phosphatidylcholine, cellulose, sterile water, syrup, and methyl cellulose. The formulations can additionally include: lubricating agents such as talc, magnesium stearate, and mineral oil; wetting agents; emulsifying and suspending agents; and preserving agents such as methyl- and propylhydroxy-benzoates and benzyl alcohol. The compositions of the present invention can be formulated so as to provide quick, sustained or delayed release of the active ingredient after administration to the patient by employing procedures known in the art.

Additional excipients suitable for formulation with the pharmacologically active bile acid compositions of the present invention include penetration enhancers and dispersion agents. Non-limiting examples of dispersion agents which allow the dispersion of drugs in tissue include hyaluronidase and collagenase. Hyaluronidase functions to augment tissue permeability and spread or dispersion of other drugs. Collagenase has been used to isolate adipocytes from subcutaneous fat and does not have lytic effects on adipocytes themselves. Additionally hyaluronidase and collagenase can facilitate healing by accelerating removal of necrotic tissue after treatment with the bile acid formulations of the present invention.

The pharmacologically active bile acid compositions of the present invention are useful for treating localized fat accumulations, including but not limited to: submental region, for example, under the chin, other facial region, the knee region, the bra-strap regions, the front and back of torso, the back of arms, lower eyelid fat herniation, accumulations on the waist, hips and other cosmetic areas, xanthelasma, lipomas and lipodistrophy, including “buffalo hump” lipodystrophy. In another embodiment, the pharmacologically active bile acid compositions of the present invention is useful for treating fat deposits associated with cellulite.

It is further contemplated that the compounds as disclosed herein can be used in various other pharmaceutical uses. For example, in one embodiment, the compounds disclosed herein may be used as an antifungal agent (U.S. Pat. No. 4,681,876), as prodrugs (U.S. 2003/0212051), to reduce hair growth (U.S. Pat. No. 7,618,956), to treat irritable bowel syndrome (U.S. 2006/0029550), to treat urinary incontinence (U.S. 2008/0254097), to treat Gram positive bacteria (U.S. 2007/0049554), to treat colorectal disorder (U.S. 2007/0072828), and to treat visual disorders (see, U.S. 2008/0194531).

The foregoing and other aspects and embodiments of this invention may be better understood in connection with the following examples.

### EXAMPLES

In the examples below and elsewhere in the specification, the following abbreviations have the indicated meanings. If an abbreviation is not defined, it has its generally accepted meaning

| | |
|---|---|
| Ac | Acetyl |
| DCM | Dichloromethane (CH₂Cl₂) |
| DMF | N,N-Dimethylformamide |
| Et | Ethyl |
| min | Minutes |
| Me | Methyl |
| Pd/C | Palladium on carbon |
| LiAl(O^{t}Bu)₃H | Lithium tri-tert-butoxyaluminum hydride |
| THF | Tetrahydrofuran |
| TLC | Thin layer chromatography |
| TBS | tert-butyl silyl |

General:

All manipulations of oxygen- and moisture-sensitive materials can be conducted with standard two-necked flame dried flasks under an argon or nitrogen atmosphere. Column chromatography can be performed using silica gel (60-120 mesh). Analytical thin layer chromatography (TLC) performed on Merck Kiesinger 60 F₂₅₄ (0.25 mm) plates. Visualization of spots can be either by UV light (254 nm) or by charring with a solution of sulfuric acid (5%) and p-anisaldehyde (3%) in ethanol.

Apparatus:

Proton and carbon-13 nuclear magnetic resonance spectra (¹H NMR and ¹³C NMR) can be recorded on a Varian Mercury-Gemini 200 (¹H NMR, 200 MHz; ¹³C NMR, 50 MHz) or a Varian Mercury-Inova 500 (¹H NMR, 500 MHz; ¹³C NMR, 125 MHz) spectrometer with solvent resonances as the internal standards (¹H NMR, CHCl₃ at 7.26 ppm or DMSO at 2.5 ppm and DMSO-H₂O at 3.33 ppm; ¹³C NMR, CDCl₃ at 77.0 ppm or DMSO at 39.5 ppm). ¹H NMR data are reported as follows: chemical shift (6, ppm), multiplicity (s=singlet, d=doublet, t=triplet, q=quartet, br=broad, m=multiplet), coupling constants (Hz), and integration. Infrared spectra (FT-IR) were run on a JASCO-460⁺ model. Mass spectra can be obtained with a Perkin Elmer API-2000 spectrometer using ES⁺ mode. Melting points were determined using a LAB-INDIA melting point measuring apparatus and are uncorrected. High-performance Liquid Chromatography (HPLC) chromatograms can be recorded using a SHIMADZU-2010 model with a PDA detector. Specific optical rotations can be determined employing a JASCO-1020 at 589 nm.

Chemicals:

Unless otherwise noted, commercially available reagents can be used without purification Anhydrous solvents can be distilled from CaH₂ or sodium/benzophenone as conventionally performed in the art.

### Example 1

### Synthesis of Intermediate 7

Step 1-a) To a solution of compound 1 in chloroform is added hydrochloric acid and formaldehyde (ca 3-5 equivalents), and the resulting solution stirred over molecular sieves for 2-16 hours until determined complete by TLC. The solvent and excess formaldehyde can then be removed under vacuum, affording compound 2. Compound 2 can be used in the next step without further purification.

Step 1-b) To a solution of compound 2 in THF is added a slight excess of ethylene-1,2-diol (ca 1.5-2 equivalents) and a catalytic amount of p-toluenesulfonic acid. The resulting solution is stirred at elevated temperature (preferably refluxing) over molecular sieves for 2-16 hours until determined complete by TLC. The mixture is then diluted with methylene chloride, washed with water, dried with MgSO₄, and filtered and the solvent removed under vacuum, affording compound 3. Compound 3 can be used in the next step without further purification.

Step 1-c) To a solution of compound 3 is added 70% tert-butyl hydroperoxide (35 equivalents and 10% sodium hypochlorite (NaOCl) (7.0 equiv; added in 7 hours duration) in ethyl acetate at 0-5° C. The resulting solution is stirred at elevated temperature (preferably refluxing) over molecular sieves for 2-16 hours until determined complete by TLC. The mixture is then diluted with methylene chloride, washed with water, dried with MgSO₄, and filtered and the solvent removed under vacuum, affording compound 3. Compound 4 can be used in the next step without further purification.

Step 1-d) 10% Pd/C is added to a solution of compound 4 in EtOAc and the resulting slurry hydrogenated with hydrogen gas in a Parr apparatus (50 psi) at 50° C. for 16 h until the reaction is determined complete by TLC. The mixture is filtered through a small plug of Celite® and the solvent removed under vacuum, providing compound 5.

Step 1-e) To a solution of compound 5 in THF is added a slight excess of NaBH₄ (portionwise). The resulting solution is stirred at ambient temperature for 1-16 hours until determined complete by TLC. The mixture is then diluted with methylene chloride, washed with water, dried with MgSO₄, filtered and the solvent removed under vacuum to provide the corresponding alcohol. To a cooled solution (0° C.) of the alcohol is added an excess of anhydrous pyridine (ca 5 equiv) followed by a slight excess of acetic anhydride (ca 2-3 equiv). The resulting solution is allowed to warm to ambient temperature over 1-16 hours and stirred until determined complete by TLC. The mixture is then diluted with methylene chloride, washed with 1M HCl, dried with MgSO₄, and filtered and the solvent removed under vacuum, affording compound 6. Compound 6 can be used in the next step without further purification.

Step 1-g) To a cooled solution of 6 (<15° C.) under an inert atmosphere is added POCl₃ dropwise over 30 minutes. The reaction is allowed to warm and stir for 2 hour at which time the reaction is cooled and anhydrous pyridine (ca 5 equiv) is added. The resulting solution is allowed to warm to ambient temperature over 1-16 hours and stirred until determined complete by TLC. The mixture is then diluted with methylene chloride, washed with 1M HCl, dried with MgSO₄, and filtered and the solvent removed under vacuum, affording compound 7. Compound 7 can be used in the next step without further purification, or can be purified using standard purification methods, such as chromatography or recrystallization techniques.

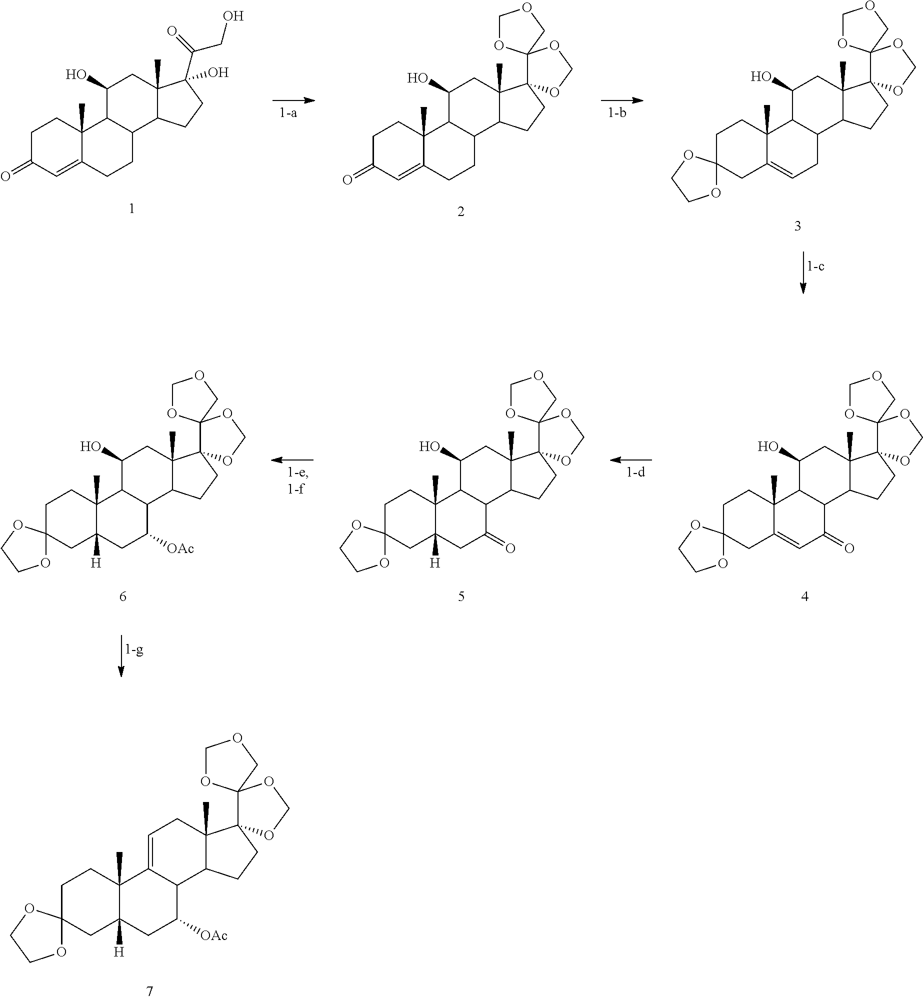

### Example 2

### Synthesis of Cholic Acid

Step 1-h) To a solution of compound 7 is added 70% tert-butyl hydroperoxide (35 equivalents and 10% sodium hypochlorite (NaOCl) (7.0 equiv; added in 7 hours duration) in ethyl acetate at 0-5 C. After work up, the organic layer is treated with sodium sulfite followed by PCC (1.0 equiv.). The residue on slurry purification in 20% aq., methanol (2 vol) provides compound 8a. Compound 8a can be used in the next step without further purification.

Step 1-d) 10% Pd/C is added to a solution of compound 8a in EtOAc and the resulting slurry hydrogenated with hydrogen gas in a Parr apparatus (50 psi) at 50° C. for 16 h until the reaction is determined complete by TLC. The mixture is filtered through a small plug of Celite® and the solvent removed under vacuum, providing compound 9.

Step 1-i) A THF solution of lithium tri-tert-butoxyaluminum hydride (1.0 M) is added to a cold (−40° C.) solution of compound 9 in THF under an inert atmosphere. The resulting reaction mixture is stirred for 2 h or until determined complete by TLC, at which time the reaction mixture is quenched with a mixture of 1N HCl and ethyl acetate, the two phases separated and the aqueous layer extracted twice with ethyl acetate. The organic phases are combined and washed with water and saturated brine solution, dried over Na₂SO₄, filtered, and evaporated to afford compound 10 which is used in the next step without purification.

Step 1-j) To a solution of compound 10 in THF is added an aqueous solution of formic acid (ca 35 equivalents), and the resulting solution stirred at ambient temperature for 2-16 hours until determined complete by TLC, at which time a mixture of 1N HCl and ethyl acetate is added, the two phases separated and the aqueous layer extracted twice with ethyl acetate. The combined organic phases are washed with water and saturated brine solution, dried over Na₂SO₄, filtered, and evaporated to afford compound 11 which is used in the next step without purification.

Step 1-k) To a solution of compound 11 in THF is added a slight excess of NaBH₄ (portionwise). The resulting solution is stirred at ambient temperature for 1-16 hours until determined complete by TLC, at which time a mixture of 1N HCl and ethyl acetate is added, the two phases separated and the aqueous layer extracted twice with ethyl acetate. The combined organic phases are washed with water and saturated brine solution, dried over Na₂SO₄, filtered, and evaporated to afford compound 12 which is used in the next step without purification.

Step 1-l) To a solution of compound 12 in THF is added a slight excess of NaBiO₄ or HIO₄ (portionwise). The resulting solution is stirred at ambient temperature for 1-16 hours until determined complete by TLC, at which time a mixture of 1N HCl and ethyl acetate is added, the two phases separated and the aqueous layer extracted twice with ethyl acetate. The combined organic phases are washed with water and saturated brine solution, dried over Na₂SO₄, filtered, and evaporated to afford compound 13 which is used in the next step without purification.

Step 1-m) A solution of potassium tert-butoxide in THF (1 M) was added drop wise to a suspension of ethyltriphenylphosphonium bromide in THF over 1 h at 25° C. The resulting dark red colored mixture is stirred for an additional 1 h at 25° C. A solution of compound 13 in THF is added slowly to the red-colored mixture at 25° C. The resulting mixture is stirred for 3-4 h until determined complete by TLC, at which time the reaction is quenched with saturated aqueous NH₄Cl, the phases were separated and the aqueous layer extracted with EtOAc. The organic fractions are combined, washed with saturated brine solution, dried over Na₂SO₄, and filtered. The filtrate is concentrated under vacuum and the crude solid purified by column chromatography (ethyl acetate/hexanes (1:9)). The fractions containing product are combined and concentrated, providing compound 14.

Step 1-n) Compound 14 is dissolved in CH₂Cl₂. Triethylamine, DMAP and acetic anhydride are added sequentially at 25° C. under a nitrogen atmosphere. The resulting solution is stirred for 2 h at 25° C. until determined by TLC to be complete. The reaction is quenched by the addition of ice-water and the phases separated. The aqueous layer is extracted with CH₂Cl₂, the organic fractions combined and washed with saturated brine solution, dried over anhydrous Na₂SO₄, and filtered. The filtrate is concentrated under vacuum to afford the triacetate of compound 14. Ethyl aluminum dichloride is added to a solution of methyl propiolate in CH₂Cl₂ at 0° C. under an inert atmosphere. The resulting solution is stirred for 15 minutes followed by the addition of triacetate of compound 14. After stirring for an additional 20 min at 0° C., the temperature is raised to 25° C. and held there for a further 18 h or until determined complete by TLC. The mixture is then poured into cold (0° C.) water, the phases separated and the aqueous layer extracted with CH₂Cl₂. The organic layers are then combined and washed sequentially with water and saturated brine solution, dried over anhydrous Na₂SO₄, and filtered. The filtrate is concentrated under vacuum to provide compound 15.

Step 1-o) PtO₂ is added to a solution of compound 15 in EtOAc and the resulting slurry hydrogenated with hydrogen gas in a Parr apparatus (50 psi) at 50° C. for 16 h until the reaction is determined complete by TLC. The mixture is filtered through a small plug of Celite® and the solvent removed under vacuum, providing compound 16a.

Step 1-p) A solution of LiOH in H₂O is added to a solution of compound 16a in THF and MeOH. The resulting mixture is stirred for 3-4 h at 50° C. until complete disappearance of the starting material by TLC. Then the reaction mixture is concentrated under vacuum. A mixture of water and 3 N HCl (10:1) is combined and cooled to 0° C. and then added to crude product. After stirring for 1 h at 0° C., the precipitated solids are filtered and washed with water and hexane (1:2). Drying under vacuum at room temperature provided cholic acid 16.

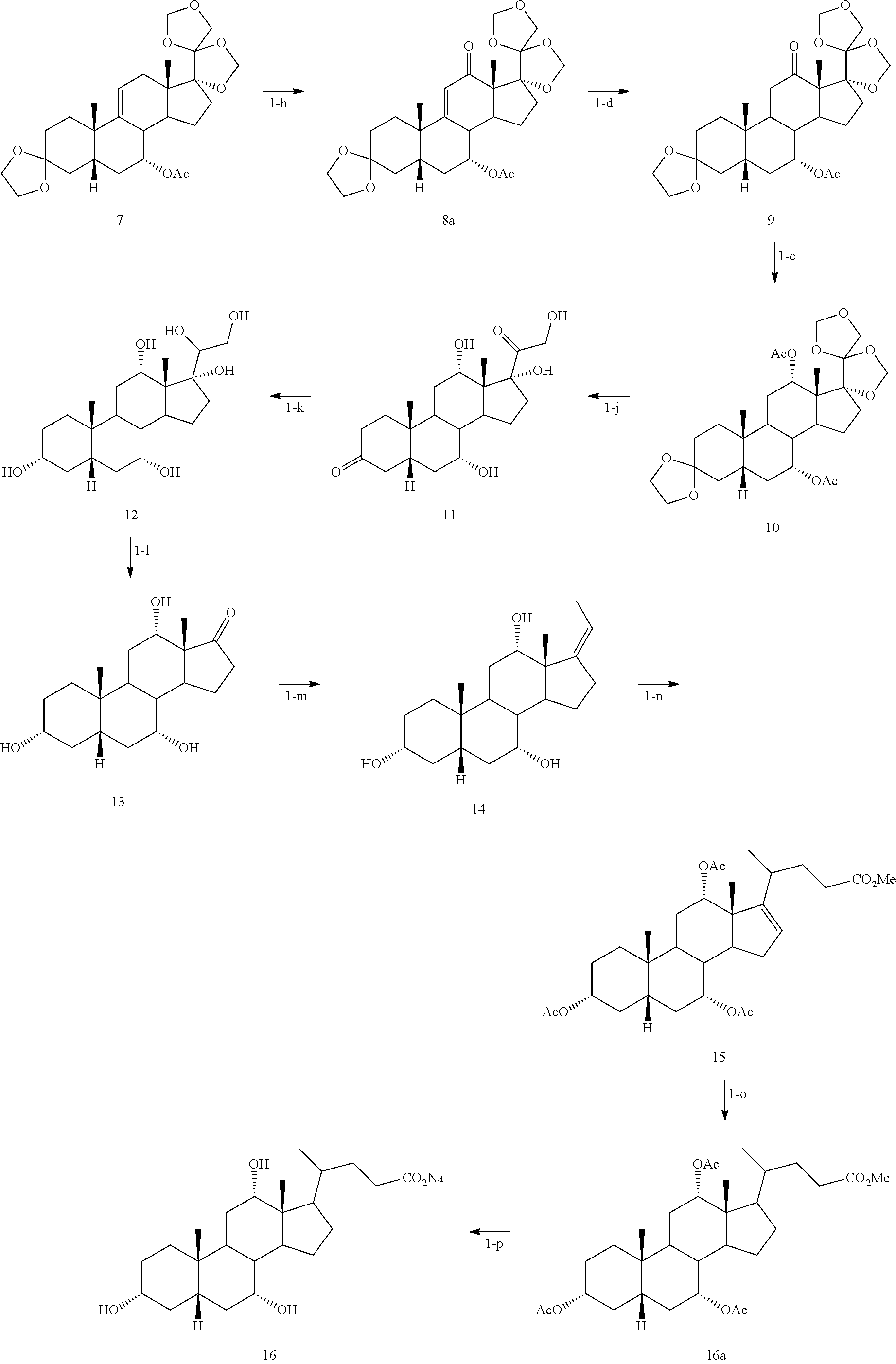

### Example 3

### Synthesis of a 12-hydroxy Estrogen Derivative by Chelation Directed Oxidation

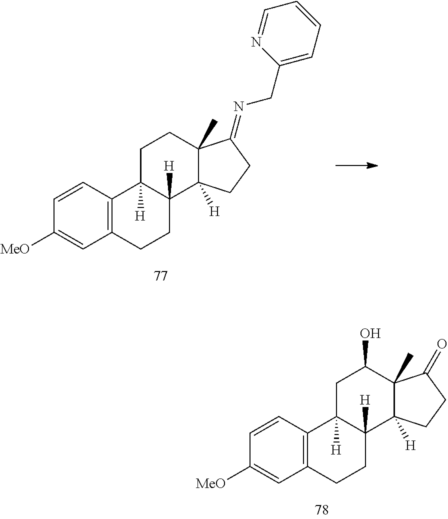

This example describes the synthesis of compound 78 which is useful for synthesizing DCA according to this invention. A solution of compound 77 (1.0 g, 2.67 mmol), which is easily synthesized from commercially available estrone methyl ether, in anhydrous dichloromethane (150 mL) is stirred continually at room temperature, and water-free copper(ii)triflate (0.97 g, 2.67 mmol) is added slowly. After 3 h, the reaction mixture is degassed with argon. Under an argon atmosphere and with continual stirring, benzoin (1.13 g, 5.34 mmol) and triethylamine (0.74 mL, 5.34 mmol) are added. After 20 h, the argon atmosphere is replaced by an O₂ atmosphere. The reaction mixture is stirred a further 3 days. Over a period of 2 h, aqueous ammonia (25% NH₃, 3×30 mL) is added under vigorous stirring. The aqueous phase is separated and extracted twice with dichloromethane. The combined organic phases are concentrated and dried over Na₂SO₄, and the solvent is removed by distillation. The residue is separated by chromatography on silica gel eluting initially with dichloromethane, and then with CH₂Cl₂/CH₃OH (95:5) to provide compound 78. Compound 78 is converted to DCA according to the methods disclosed here, which include, without limitation, reducing the aromatic ring, incorporating the 19-angular methyl (on the C-10), and oxidizing the 12-beta hydroxy group followed by reducing the 12-oxo group to a 12-alpha hydroxy group, incorporating the side chain via Witting reaction and metathesis reactions and following methods well known to the skilled artisan.

### Example 4

### 9-hydroxylation of Estrogen Derivatives

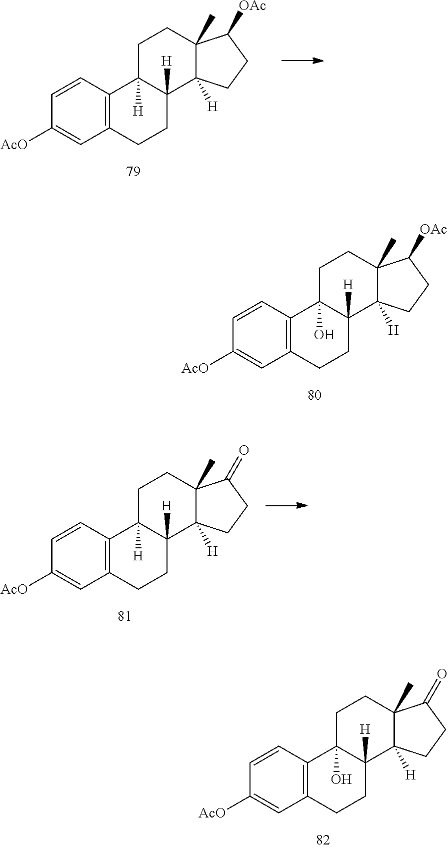

This example describes the synthesis of compounds 80 and 82, which are useful for synthesizing DCA according to this invention. To a solution of the diacetate 79 (0.3 g) in dichloromethane (20 ml) is added NBu₄HSO₄ (0.1 g) acetone (10 ml) and phosphate buffer (pH 7.5, 25 ml). The mixture is cooled to 2° C. and the pH is adjusted to 7.5. A solution of KHSO₅ (9 g) and Na₂EDTA (0.2 g) in distilled water (60 ml) is added dropwise over 7 h and the mixture is stirred for a further 17 h while maintaining the temperature at 0-5° C. and the pH at 7.5. The dichloromethane solution is separated, dried (MgSO₄) and evaporated in vacuo to afford the crude product (0.393 g) which is separated by flash chromatography on silica gel eluting with diethyl ether-light petroleum (b.p. 40-60° C.)-ethyl acetate (15:10:1) to afford the 9-hydroxy compound 80. Compound 82 is similarly synthesized from compound 81.

Compounds 80 and 82 are converted to DCA according to the methods disclosed here, which include, without limitation, appropriately protecting the 17 hydroxy or 17-oxo group, dehydrating to provide the delta-9,11-ene compound, oxidizing the 9,11-ene compound to an alpha beta 9,11-ene-12-one or a 9,11-ene-12-hydroxy compound, reducing the 9,11-double bond, reducing the aromatic ring, incorporating the 19-angular methyl, and oxidizing the 12-beta hydroxy group followed by reducing the 12-oxo group to a 12-alpha hydroxy group, incorporating the side chain via Witting reaction and metathesis reactions, and following methods well known to the skilled artisan.

### Example 5

### Preparation of 9-hydroxy androst-4-ene-3,17-dione from Androstenedione

A pre-seed is prepared by taking a loopful of biomass from a slant of Nocardia canicruria ATCC 31548 and inoculating it into 50 ml of Tryptic Soy Broth (TSB) in a 200 ml Erlenmeyer flask and then incubating it on a 30° C. shaker for 40 hours. A seed is prepared by taking 5 ml of the above described pre-seed and transferring it into a 2.8 liter fernbach flask containing a liter of TSB. The fernbach is incubated on a 30° C. shaker for 31 hours. A seed tank medium is prepared by combining the following ingredients to yield 42 liters: dextrose 2.5 g/l 105 g/tank, K₂HPO₄2.5 g/l 105 g/tank, HY-CASE 15.0 g/1630 g/tank, HY-SOY 5.0 g/1210 g/tank, 30% silicone antifoam agent 0.25 g/l 10.5 g/tank. pH is maintained at approximately 7.3 to 7.5 and sterilization time is approximately 45 minutes at 120° C. The temperature of the seed tank is kept at 30° C. with 10 PSI and constant air flow. Androstenedione (25 g) is dissolved in approximately 200 milliliters of methanol. The methanol solution is then added to 1 liter of sterile water in a 2.8 liter fernbach flask. The suspension is then pasteurized and injected into the seed tank. The seed tank is then inoculated with 5 percent of the seed solution described above and inoculated. The seed tank is then extracted with two gallons of methylene chloride after 47 hours. The methylene chloride solution from each tank is then separately collected and flash evaporated to dryness. Yield 24.31 grams crude extract. The crude extract is then dissolved in 170 milliliters of methylene chloride. The solution is loaded into a 50 by 600 millimeter column containing 650 grams silica gel. The column is eluted successively with 20:80::ethyl acetate:methylene chloride, 30:70::ethyl acetate:methylene chloride, and 50:50::ethyl acetate:methylene chloride.

The initial flow rate is 500 milliliters per minute. Fractions of 500 milliliters volume are collected. The fractions are monitored by TLC. The plates are then developed using a solvent system consisting of 100 percent ethyl acetate. The desired product is eluted with a solvent system of 20:80, ethyl acetate:methylene chloride to give 9-hydroxyandrost-4-ene-3,17-dione in a yield of 45 percent. The desired product is recrystallized from methanol.

### Example 6

### Site Selective Halogenation and Dehydrohalogenation to Provide delta-9,11-ene Steroids

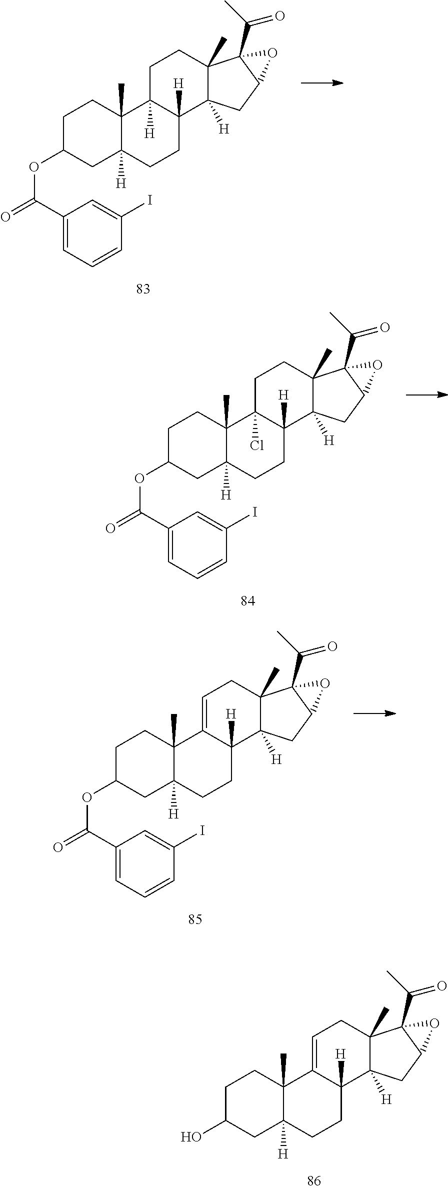

This example describes synthesizing compounds 84, 85, and 86, which are useful for synthesizing DCA according to this invention. A 500 mg (0.9 mmol) amount of the m-iodobenzoate 83 is dissolved in 90 ml of redistilled dichloromethane. Iodobenzene dichloride (300 mg, 1.08 mmol, 1.2 mol-eq) is added. The solution is degassed by a series of freeze thaw cycles and photolyzed with the Hanovia lamp using a Uranium glass filter for 1 h. The solution is kept at a temperature of 10-20° C. by using an ice-water bath. The solution is evaporated to dryness to provide an oil, including product 84. The crude photolysis product is taken up in 10 ml of dioxane and 10 ml of 10% KOH in methanol is added. The solution is refluxed for 2 h and diluted with water. The mixture is extracted with dichloromethane, washed with water, dried, and evaporated to give 240 mg of crude product 85, which is purified by kieselgel column chromatography with hexane-ether mixture (1:2 volume/volume) to give the pure enone 86. Compound 86 is converted to DCA according to the methods disclosed here, which include, without limitation, oxidizing compound 10 to an alpha beta 9,11-ene-12-one or a 9,11-ene-12-hydroxy compound, reducing the 9,11-double bond, converting the A-B ring junction to be cis, and oxidizing the 12-beta hydroxy group followed by reducing the 12-oxo group to a 12-alpha hydroxy group, incorporating the side chain via Witting reaction and metathesis reactions, and following methods well known to the skilled artisan.

### Example 7

### Angular Methylation of 1,4-dihydroestrone Derivative

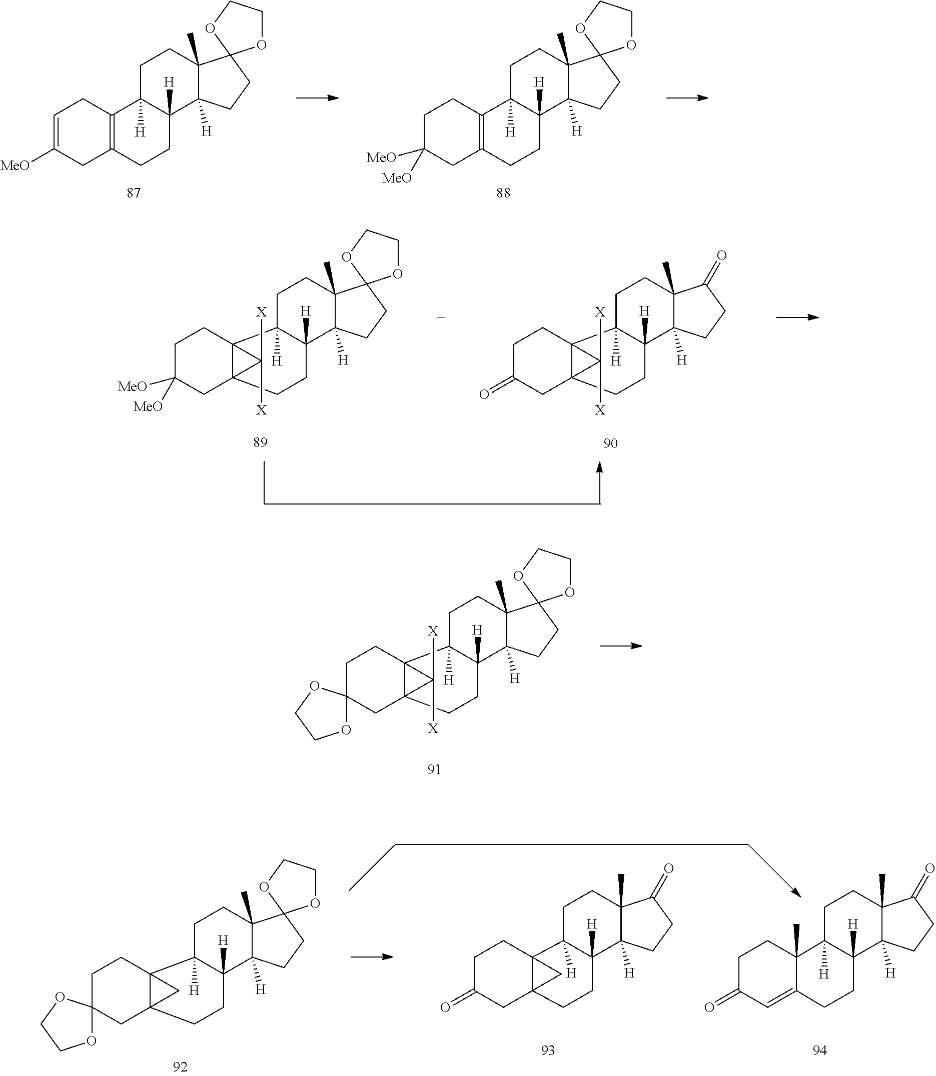

This example describes the step wise incorporation of a 19-angular methyl into a Birch-reduced estrogen derivative. 1,4-Dihydroestron-3-methyl ether-17-ketal (compound 87, 1 g), in dry ether (50 mL) and methanol (1 mL), is cooled to 0° and a crystal of toluene-p-sulphonic acid is added. The mixture is left at 0° for 2 hr, refluxed for 30 min, neutralized with sodium methoxide, washed with water, and dried. Removal of the solvent and crystallization of the residue from methanol provides 3,3-dimethoxyestr-5(10)-ene 17-ketal (compound 88).

To a mixture of compound 88 (800 mg) and potassium t-butoxide (1 g) in dry ether (30 mL) is added dropwise a solution of bromoform (2.5 g) in ether (10 mL) at −20°, with stirring, under nitrogen. The mixture is stirred for 2 hr and left to warm to room temperature. Water (50 mL) is added and the contents are extracted with chloroform (3×30 mL), washed thoroughly with water, and dried. Removal of the solvent provides a mixture of ketone and the ketal, which is deketalized with toluene-p-sulphonic acid and worked up in the usual way, to give a semi-solid mass that is separated by column chromatography on alumina to give the dibromo-dione (compound 90).

Compound 90 (100 mg) is re-ketalized by refluxing with ethylene glycol and toluene-p-sulphonic acid in anhydrous toluene. Working up as usual gives a glassy mass of the diketal (compound 91), which is reduced with lithium (20 mg), liquid ammonia (30 mL), and ethanol (2 mL). The ammonia is allowed to evaporate and water (25 mL) is added. The product is extracted with light petroleum (b p. 40-60°; 3×10 mL), washed thoroughly with water, and dried. Removal of the solvent gives a liquid which yields 5,10-methyleneestrane 3,17-diethylene ketal (compound 92). Compound 15 is deketalized with toluene-p-sulphonic acid in acetone and the resulting 5,10-methyleneestrane-3,17-dione (compound 93).

A stream of dry hydrogen chloride is passed through a solution of 5,10-methylenerestrane 3,17-diketal (compound 92, 50 mg) in dry chloroform (10 mL) for 1 hr. The mixture is left overnight, and working up as usual gives a residue, which is separated by column chrotagraphy on alumina to provide androst-4-ene-3,17-dione (compound 94).

A 12-hydroxy or a 12-oxo estrone derivative is similarly converted into a 12-hydroxy or 12-oxo androst-4-ene-3,17-dione.

1. A method for preparing a compound of formula 7 or a pharmaceutically acceptable salt thereof: 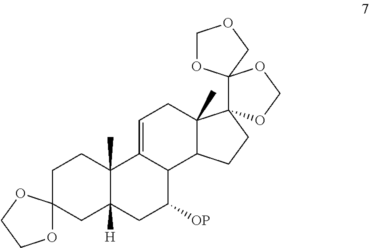
said method comprising
(a) contacting hydrocortisone 1 with formaldehyde to form compound 2 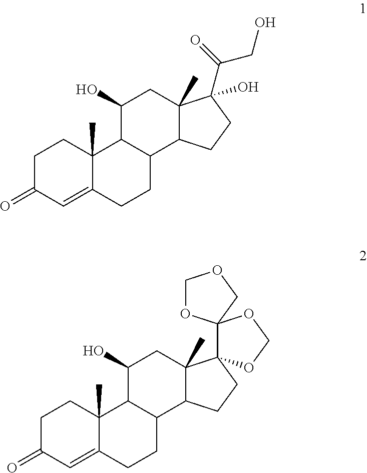
(b) contacting compound 2 with ethane-1,2-diol to form compound 3 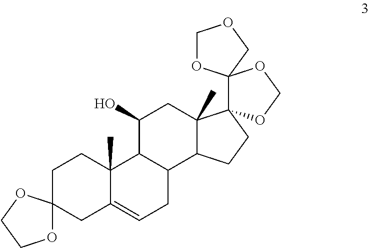
(c) contacting compound 3 with an oxidizing agent to form compound 4 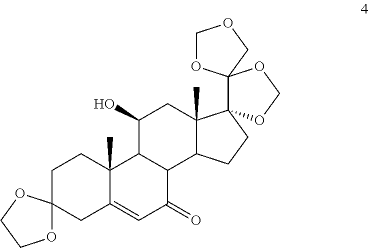
(d) contacting compound 4 under hydrogenation conditions to form compound 5 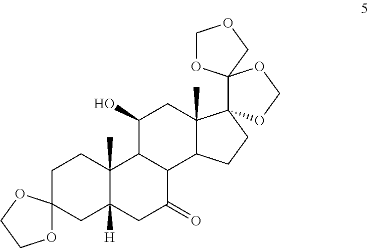
(e) contacting compound 5 with a reducing agent to form compound 6a 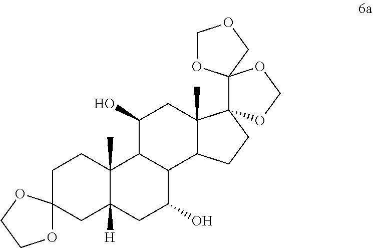
(f) converting compound 6a to compound 6 wherein P is a protecting group 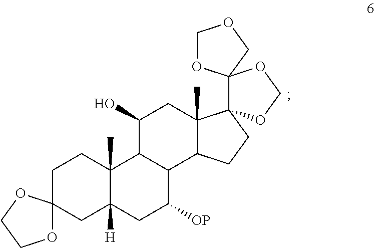
and
(g) contacting compound 6 under elimination conditions to form compound 7.

2. A method for preparing a compound of formula 8a: 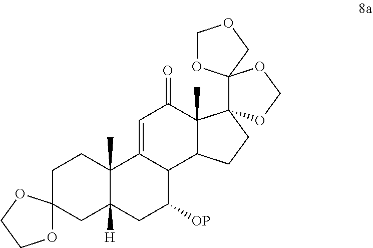
said method comprising:
contacting a compound of formula 7 under oxidizing conditions to form a compound of formula 8a wherein P is a protecting group

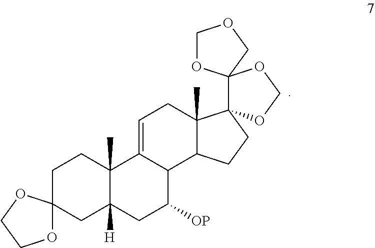

3. A method for preparing a compound of formula 9: 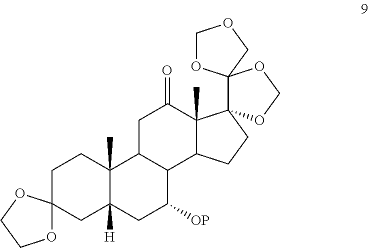
said method comprising:
contacting a compound of formula 7 under oxidizing conditions to form a compound of formula 8a wherein P is a protecting group 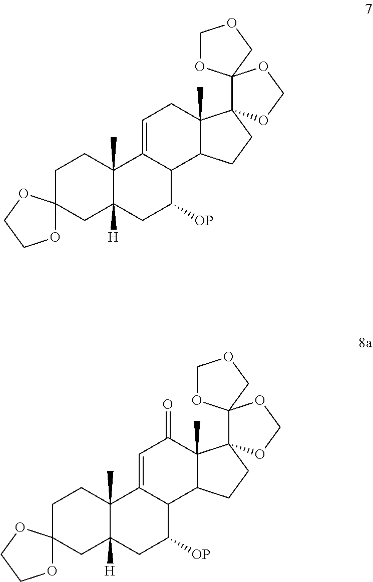
contacting a compound of formula 8a with H₂ under hydrogenation conditions to form compound 9 wherein P is a protecting group.

4. A method for preparing cholic acid 16 or a pharmaceutically acceptable salt thereof: 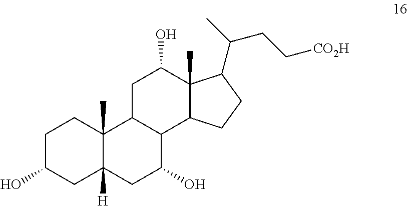 [SMILES: CC(CCC(=O)O)C1CCC2C3C(C[C@H](O)[C@]12C)[C@@]1(C)CC[C@@H](O)C[C@H]1C[C@H]3O]
said method comprising
(a) contacting hydrocortisone 1 with formaldehyde to form compound 2 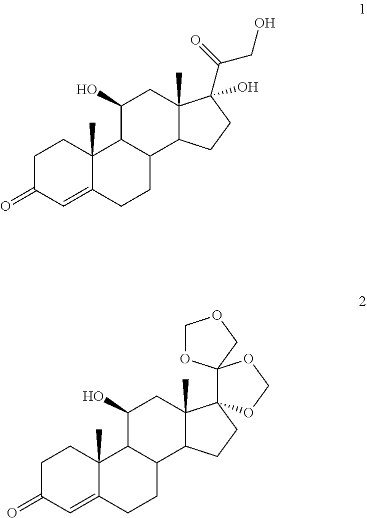
(b) contacting compound 2 with ethane-1,2-diol to form compound 3 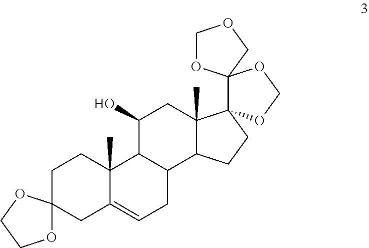
(c) contacting compound 3 under oxidizing conditions to form compound 4 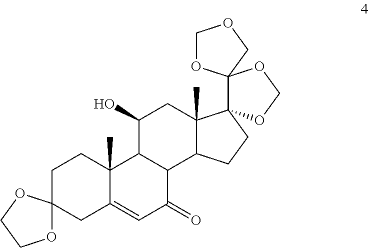
(d) contacting compound 4 under hydrogenation conditions to form compound 5 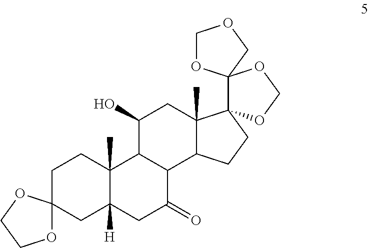
(e) contacting compound 5 under reducing conditions to form compound 6a 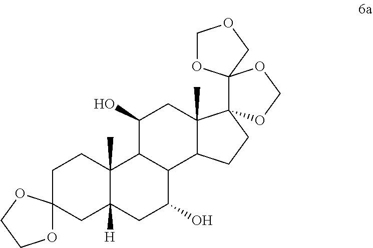
(f) converting compound 6a to compound 6 wherein P is a protecting group 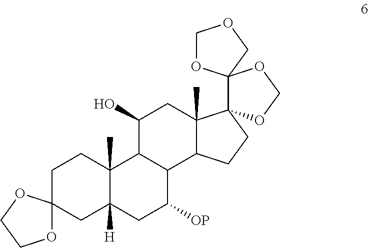
(g) contacting compound 6 under elimination conditions to form compound 7 wherein P is a protecting group 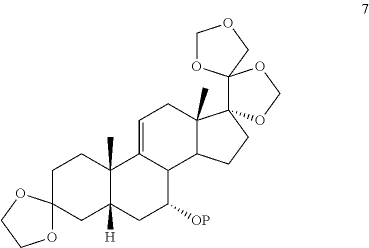
(h) contacting compound 7 under oxidizing conditions to form compound 8a wherein P is a protecting group 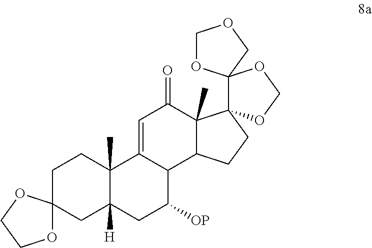
(i) contacting compound 8a under hydrogenation conditions to form compound 9 wherein P is a protecting group 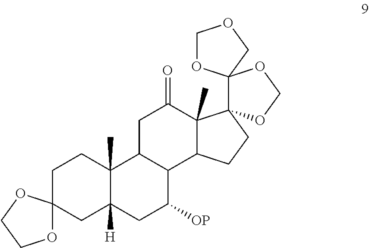
(j) contacting compound 9 under reducing conditions to form compound 10 wherein P is a protecting group 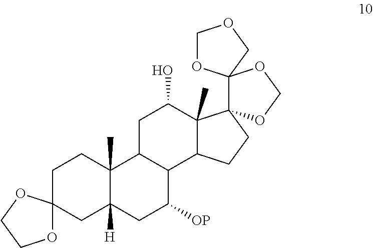
(k) contacting compound 10 to form compound 11 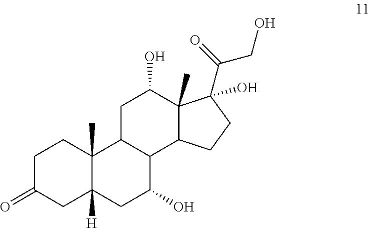 [SMILES: C[C@]12CCC(=O)C[C@H]1C[C@@H](O)C1C2C[C@H](O)[C@@]2(C)C1CC[C@]2(O)C(=O)CO]
(l) contacting compound 11 under reducing conditions to form compound 12 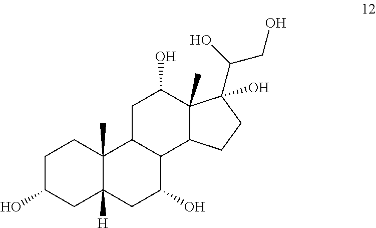 [SMILES: C[C@]12CC[C@@H](O)C[C@H]1C[C@@H](O)C1C2C[C@H](O)[C@@]2(C)C1CC[C@]2(O)C(O)CO]
(m) contacting compound 12 with a vicinal alcohol oxidizing agent to form compound 13 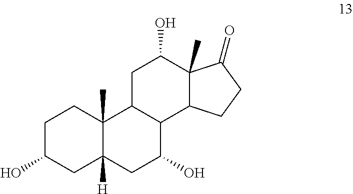 [SMILES: C[C@]12CC[C@@H](O)C[C@H]1C[C@@H](O)C1C2C[C@H](O)[C@]2(C)C(=O)CCC12]
(n) contacting compound 13 with a two carbon olefination reagent under olefin forming conditions to form compound 14 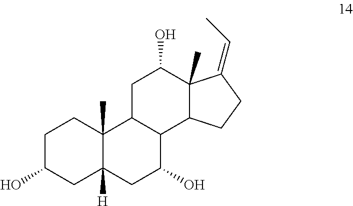 [SMILES: C/C=C1/CCC2C3C(C[C@H](O)[C@]12C)[C@@]1(C)CC[C@@H](O)C[C@H]1C[C@H]3O]
(o) contacting a compound of formula 14 with an alkyl propiolate CHCC(O)OR¹⁰ wherein R¹⁰ is alkyl in the presence of a Lewis acid to form a compound of formula 15; 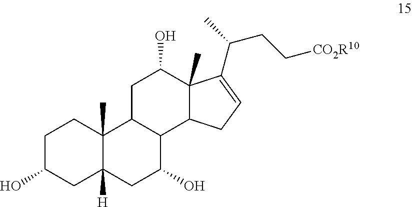
(p) contacting compound 15 under hydrogenation conditions to form 16a 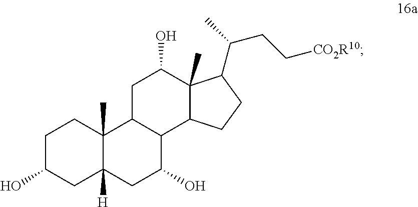
and
(q) exposing compound 16a to hydrolysis conditions to form cholic acid 16.

5. A method for preparing chenodeoxycholic acid 23 or a pharmaceutically acceptable salt thereof: 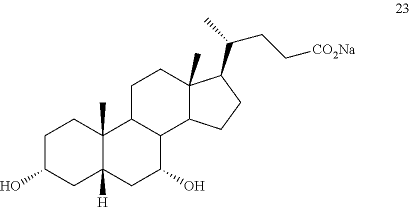
said method comprising:
(a) contacting compound 7 with an acid to form compound 17 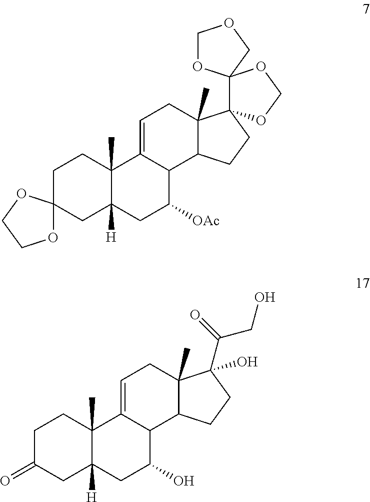
(b) contacting compound 17 with a reducing agent to form compound 18 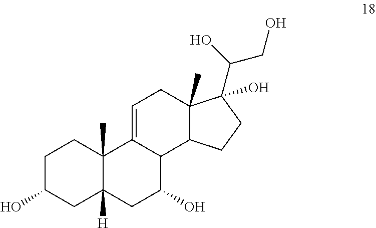 [SMILES: C[C@]12CC=C3C(C1CC[C@]2(O)C(O)CO)[C@H](O)C[C@@H]1C[C@H](O)CC[C@]31C]
(c) contacting compound 18 with a vicinal alcohol oxidizing agent to form compound 19 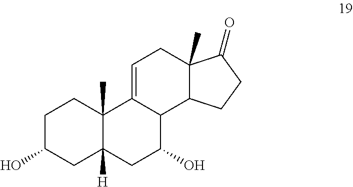 [SMILES: C[C@]12CC=C3C(C1CCC2=O)[C@H](O)C[C@@H]1C[C@H](O)CC[C@]31C]
(d) contacting compound 19 with a two carbon olefination reagent under olefin forming conditions to form compound 20 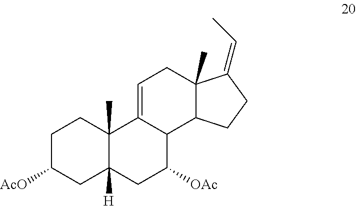 [SMILES: C/C=C1/CCC2C3C(=CC[C@]12C)[C@@]1(C)CC[C@@H](OC(C)=O)C[C@H]1C[C@H]3OC(C)=O]
(e) contacting a compound of formula 20 with an alkyl propiolate CHCC(O)OR¹⁰ or an alkyl acrylate CH₂═CHC(O)OR¹⁰ wherein R¹⁰ is alkyl in the presence of a Lewis acid to form a compound of formula 21 wherein R¹⁰ is a alkyl, and the dashed line [symbol] is a single or double bond; 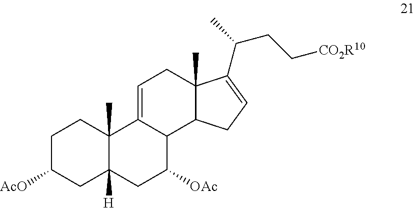
(f) contacting compound 21 with H₂ under hydrogenation conditions to form compound 23a 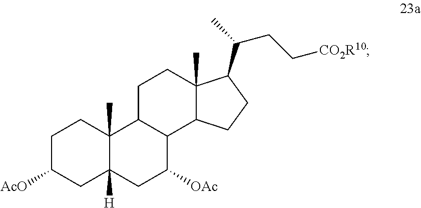
and
(g) exposing compound 23a to hydrolysis conditions to form chenodeoxycholic acid 23.

6. A method for preparing lithocholic acid 30 or a pharmaceutically acceptable salt thereof: 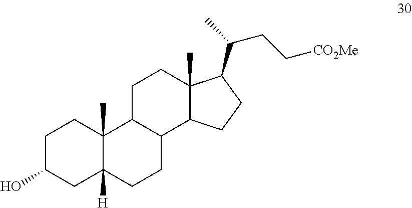 [SMILES: COC(=O)CC[C@@H](C)[C@H]1CCC2C3CC[C@@H]4C[C@H](O)CC[C@]4(C)C3CC[C@@]21C]
said method comprising:
(a) contacting compound 23a with an acid to form compound 26 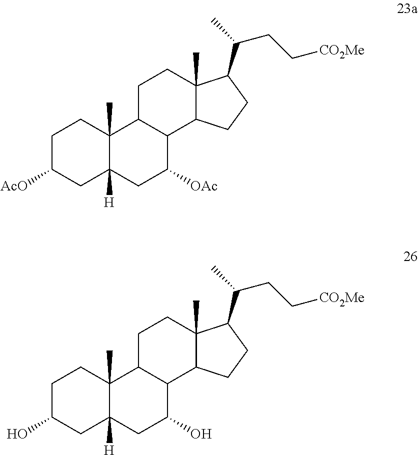 [SMILES: COC(=O)CC[C@@H](C)[C@H]1CCC2C3C(CC[C@@]21C)[C@@]1(C)CC[C@@H](OC(C)=O)C[C@H]1C[C@H]3OC(C)=O ; COC(=O)CC[C@@H](C)[C@H]1CCC2C3C(CC[C@@]21C)[C@@]1(C)CC[C@@H](O)C[C@H]1C[C@H]3O]
(b) converting compound 26 to compound 27 wherein P is a protecting group 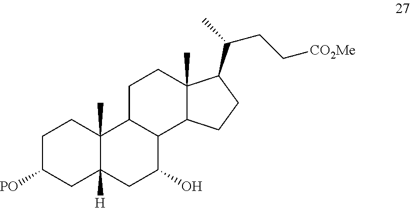
(c) contacting compound 27 under deoxygenating conditions to form compound 28 wherein P is a protecting group 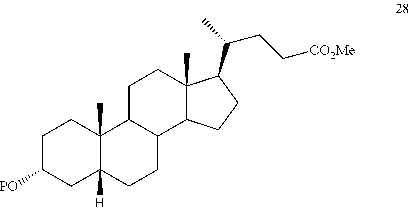
(d) contacting compound 28 under acidic conditions to form compound 29 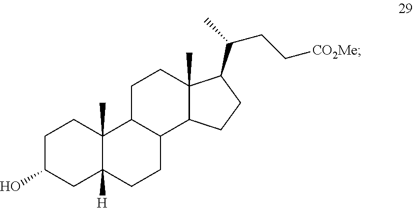 [SMILES: COC(=O)CC[C@@H](C)[C@H]1CCC2C3CC[C@@H]4C[C@H](O)CC[C@]4(C)C3CC[C@@]21C]
and
(e) exposing compound 29 to hydrolysis conditions to form lithocholic acid 30.

7. A method for preparing a compound of formula VIIB or a pharmaceutically acceptable salt thereof: 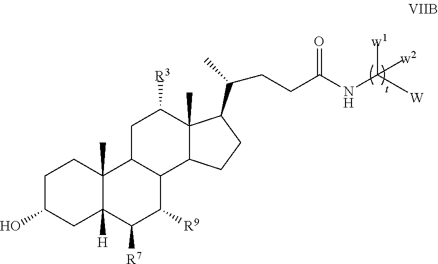
wherein R³ and R⁹ independently are hydrogen or hydroxy;
R⁷ is hydrogen, halo, C₁-C₄ alkyl, C₁-C₄ alkylene, C₁-C₄ alkyne, C₁-C₄ alkoxy;
t is 1 or 2,
w¹ and w² are each independently H or (C₁₋₄)alkyl optionally substituted with hydroxy, alkoxy, thio, thioalkyl, amino, substituted amino, aryl, and substituted aryl, and
W is —COOH or —SO₃H;
said method comprising:
contacting compound VIIA with a compound of formula VIIC under coupling conditions

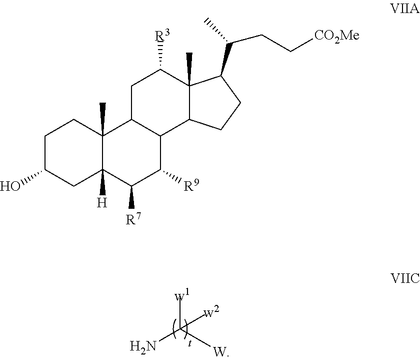

8. (canceled)

9. A method for preparing a compound of formula VIII or a pharmaceutically acceptable salt thereof: 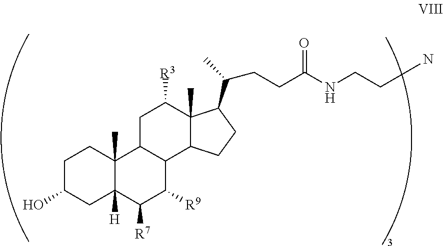
wherein R⁷ is hydrogen, halo, C₁-C₄ alkyl, C₁-C₄ alkylene, C₁-C₄ alkyne, C₁-C₄ alkoxy;
said method comprising:
contacting compound VIIA 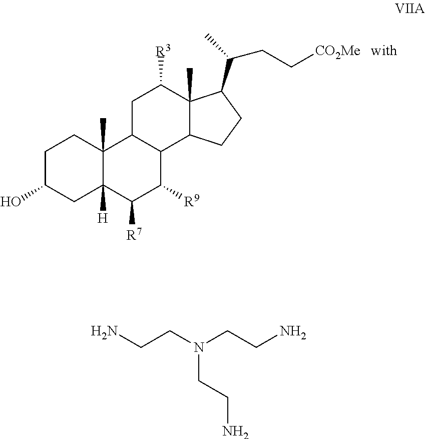
under coupling conditions.

10. (canceled)

11. A method for preparing a compound of formula 70 or a pharmaceutically acceptable salt thereof: 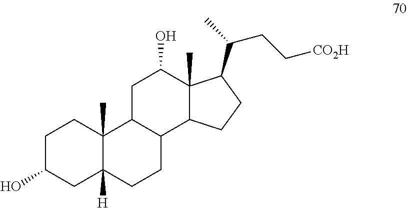 [SMILES: C[C@H](CCC(=O)O)[C@H]1CCC2C3CC[C@@H]4C[C@H](O)CC[C@]4(C)C3C[C@H](O)[C@@]21C]
said method comprising:
(a) contacting compound 3 with H₂ under conditions to form compound 60 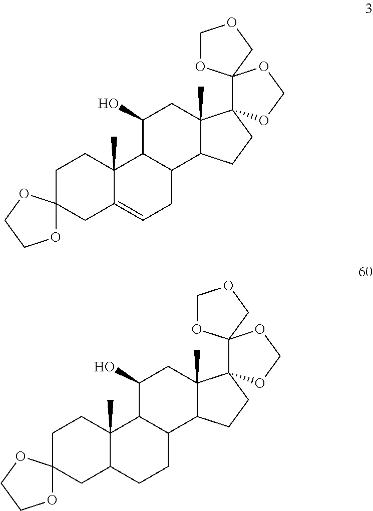
(b) contacting compound 60 under elimination conditions to form compound 61 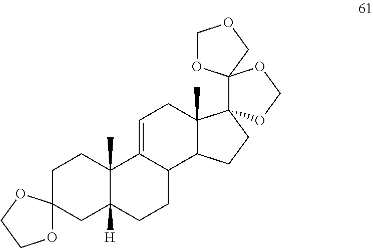
(c) contacting compound 61 with an oxidizing agent to form compound 62 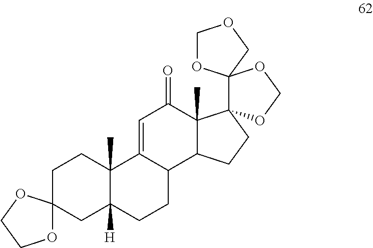
(d) contacting compound 62 with H₂ under conditions to form compound 63 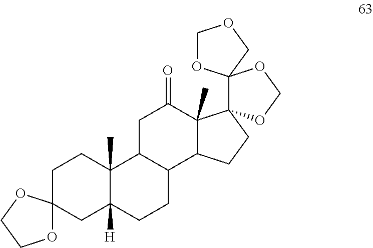
(e) contacting compound 63 with a reducing agent under conditions to form compound 64 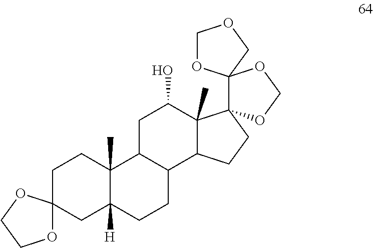
(f) contacting compound 64 with an acid to form compound 65 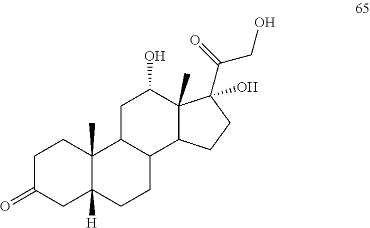 [SMILES: C[C@]12CCC(=O)C[C@H]1CCC1C2C[C@H](O)[C@@]2(C)C1CC[C@]2(O)C(=O)CO]
(g) contacting compound 65 with a reducing agent under reducing conditions to form compound 66 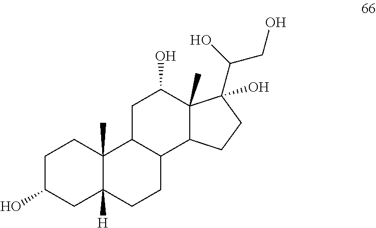 [SMILES: C[C@]12CC[C@@H](O)C[C@H]1CCC1C2C[C@H](O)[C@@]2(C)C1CC[C@]2(O)C(O)CO]
(h) contacting compound 66 with a vicinal alcohol oxidizing agent to form compound 67 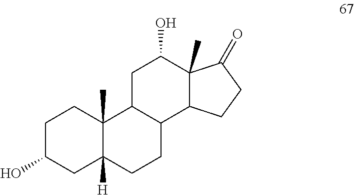 [SMILES: C[C@]12CC[C@@H](O)C[C@H]1CCC1C2C[C@H](O)[C@]2(C)C(=O)CCC12]
(i) contacting compound 67 with a two carbon olefination reagent under olefin forming conditions to form compound 68 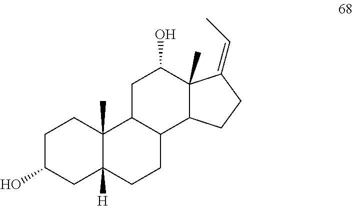 [SMILES: C/C=C1/CCC2C3CC[C@@H]4C[C@H](O)CC[C@]4(C)C3C[C@H](O)[C@]12C]
(j) contacting a compound of formula 68 with an alkyl propiolate CH≡CC(O)OR¹⁰ or an alkyl acrylate CH₂═CHC(O)OR¹⁰ wherein R¹⁰ is C₁-C₆ alkyl in the presence of a Lewis acid to form a compound of formula 69 wherein the dashed line [symbol] is a single or double bond; 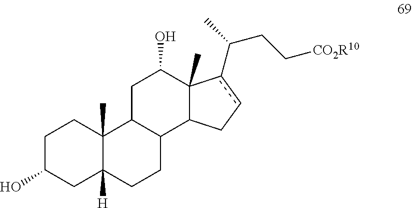
(k) contacting compound 69 wherein the dashed line [symbol] is a double bond with H₂ under hydrogenation conditions to form 70a 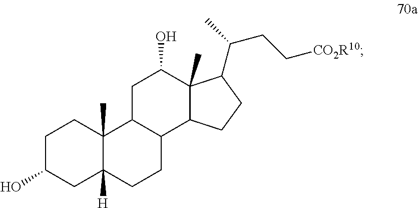
and
(l) exposing compound 70a to hydrolysis conditions to form deoxycholic acid 70.

12. A method of synthesis comprising contacting a compound of formula: 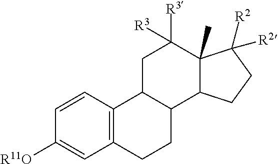
wherein R¹¹ is substituted or unsubstituted alkyl;
wherein R² and R^{2′} are independently H and OR²², provided that one of R² and R^{2′} is OR²², or CR²R^{2′} is oxo, or R² and R^{2′} together with the carbon atom they are attached form a cyclic ketal;
R²² is H or substituted or unsubstituted alkyl, alkenyl, alkynyl, or aryl;
R³ and R^{3′} are independently H and OR³¹, provided that one of R³ and R^{3′} is OR³¹; or
CR³R^{3′} is oxo;
R³¹ is H or substituted or unsubstituted alkyl or alkenyl;
under reducing conditions to provide a compound of formula:

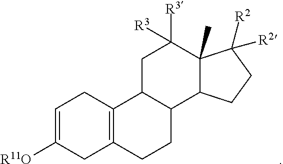

13-21. (canceled)

22. A synthetic bile represented by formula VII: 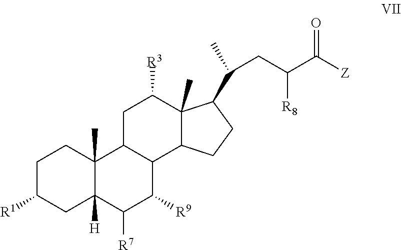
wherein:
R¹, R³, and R⁹ are each independently hydrogen, hydroxy, or C₁-C₄ alkoxy;
R⁷ is hydrogen, halo, C₁-C₄ alkyl, C₁-C₄ alkylene, C₁-C₄ alkyne, C₁-C₄ alkoxy;
R⁸ is hydrogen, halo, C₁-C₄ alkyl, C₁-C₄ alkylene, C₁-C₄ alkyne, C₁-C₄ alkoxy, haloalkyl;
Z is hydroxy, alkoxy, —NH₂, or 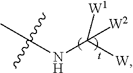
where t is 1 or 2, w¹ and w² are each independently H or (C₁₋₄)alkyl optionally substituted with hydroxy, alkoxy, thio, thioalkyl, amino, substituted amino, aryl, and substituted aryl, and W is —COOH or —SO₃H; or
a salt thereof;
provided that when R⁷ is hydrogen and R⁹ and Z are hydroxy, then R³ is not hydroxy.

23. A compound according to formula 3:

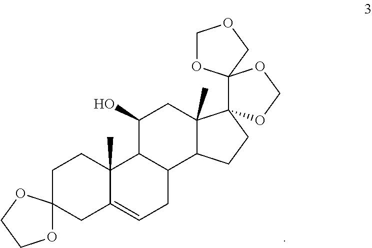

24. A compound according to formula 7:

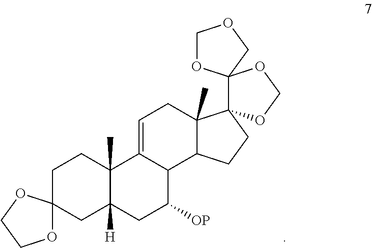

25. A compound according to formula 23a: 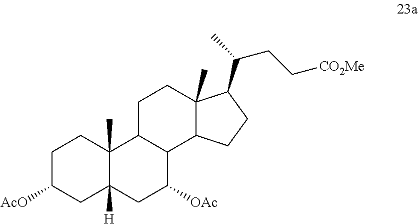
where Ac is CH₃C(O)—.

26-44. (canceled)
